(19) United States
(12) Patent Application Publication
(10) Pub. No.: US 2026/0242732 A1
(43) Pub. Date: Aug. 20, 2026

(54) MICROENVIRONMENT ACTUATED T-CELLS AND USES THEREOF

(71) Applicants: Memorial Sloan-Kettering Cancer Center, New York, NY (US); Memorial Hospital for Cancer and Allied Diseases, New York, NY (US); Sloan-Kettering Institute for Cancer Research, New York, NY (US)

(72) Inventors: Pedro Silberman, New York, NY (US); Kristen Vogt, New York, NY (US); David A. Scheinberg, New York, NY (US); James Han, New York, NY (US)

(21) Appl. No.: 19/470,720

(22) PCT Filed: Mar. 22, 2024
(86) PCT No.: PCT/US2024/021186

§ 371 Date: Sep. 29, 2025

Related U.S. Application Data
(60) Provisional application No. 63/456,336, filed on Mar. 31, 2023.
(60) Provisional application No. 63/511,803, filed on Jul. 3, 2023.

(51) Int. Cl. C12N 5/0783; A61K 40/11; A61K 40/31; A61K 40/42; A61P 35/00; C07K 16/28; C07K 16/30; C12N 15/86

(52) U.S. Cl. CPC C12N 5/0636; A61K 40/11; A61K 40/31; A61K 40/4202; A61K 40/4211; A61K 40/4258; A61P 35/00; C07K 16/2803; C07K 16/2854; C07K 16/3084; C12N 15/86; C07K 2317/622; C12N 2510/00; C12N 2740/15043

(57) ABSTRACT

Disclosed herein are engineered T cells comprising (1) a chimeric receptor polypeptide comprising (a) an extracellular domain that specifically binds P-selectin; (b) a heterologous receptor polypeptide comprising one or more ligand-inducible proteolytic cleavage sites; and (c) an intracellular domain (ICD) comprising a transcriptional activator, wherein binding of the extracellular domain to P-selectin induces cleavage of the heterologous receptor polypeptide at the one or more ligand-inducible proteolytic cleavage sites to release the ICD, and (2) a CAR polypeptide encoded by a nucleic acid that is operably linked to a promoter that is responsive to the transcriptional activator of the released ICD.

### CROSS-REFERENCE TO RELATED APPLICATIONS

This application is the U.S. National Phase application of International Application No. PCT/US2024/021186, filed Mar. 22, 2024, which claims the benefit of and priority to U.S. Provisional Patent Application No. 63/456,336, filed Mar. 31, 2023, and U.S. Provisional Patent Application No. 63/511,803, filed Jul. 3, 2023, the contents of which are incorporated herein by reference in their entireties.

### STATEMENT OF GOVERNMENT SUPPORT

This invention was made with government support under CA023766, CA055349, CA008748, and CA241894 awarded by the National Institutes of Health. The government has certain rights in the invention.

### SEQUENCE LISTING

The contents of the .xml file submitted electronically herewith are incorporated herein by reference in their entirety, filename: 115872-3341_SL.xml; Size: 103,180 bytes; and Date of Creation: Apr. 30, 2024.

### TECHNICAL FIELD

The present disclosure provides microenvironment actuated T (MEAT) cells comprising nucleic acids encoding (1) a P-selectin-specific chimeric receptor polypeptide comprising (a) an extracellular domain that specifically binds P-selectin; (b) a heterologous receptor polypeptide comprising one or more ligand-inducible proteolytic cleavage sites; and (c) an intracellular domain comprising a transcriptional activator, wherein binding of the extracellular domain to P-selectin induces cleavage of the heterologous receptor polypeptide at the one or more ligand-inducible proteolytic cleavage sites to release the intracellular domain, and (2) a chimeric antigen receptor (CAR) polypeptide that is operably linked to a promoter that is responsive to the transcriptional activator of the released intracellular domain of the P-selectin-specific chimeric receptor polypeptide. Also disclosed herein are methods of using MEAT cells of the present disclosure to treat cancer and mitigate toxicities associated with CAR T cell therapy.

### BACKGROUND

The following description of the background of the present technology is provided simply as an aid in understanding the present technology and is not admitted to describe or constitute prior art to the present technology.

Chimeric Antigen Receptor (CAR) T-cell therapy is a promising therapeutic approach in the fight against cancer. CAR T-cells kill potently upon CAR target recognition. CD19-targeted CAR T-cells have already shown high efficacy in the treatment of patients with CD19 expressing B-cell malignancy. However, challenges of limited antigen choices for other cancers and tumor antigen escape remain. Unfortunately, there are few known antigens selectively expressed on tumors and not on normal tissues. Current CAR T-cells are designed to target one upregulated tumor antigen which is also found in low levels in normal cells. Therefore, CAR T-cells developed to target tumors can kill both tumor cells and normal cells expressing the target antigen as seen in CD19 reactive CAR T-cell treatments. In addition, on-target, off-tumor toxicities have led to failed clinicals of HER2-reactive CAR T-cells in the treatment of solid tumors and toxicity in animal studies of GD2 and ROR1 reactive CAR T-cells. Ma et al., Int J Biol Sci. 2019; 15: 2548-60; Newick et al., Annu Rev Med. 2017; 68:139-52. Therefore, systemic targeting of tumor associated antigens pose significant risks to cancer patients and is a major limiting factor for future development of CAR T-cell therapeutics.

Accordingly, there is an urgent need for therapeutic compositions and methods that improve the efficacy of CAR-T cell therapy.

### SUMMARY OF THE PRESENT TECHNOLOGY

The present disclosure provides microenvironment actuated T (MEAT) cells comprising nucleic acids encoding (1) a P-selectin-specific chimeric receptor polypeptide comprising (a) an extracellular domain that specifically binds P-selectin; (b) a heterologous receptor polypeptide comprising one or more ligand-inducible proteolytic cleavage sites; and (c) an intracellular domain comprising a transcriptional activator, wherein binding of the extracellular domain to P-selectin induces cleavage of the heterologous receptor polypeptide at the one or more ligand-inducible proteolytic cleavage sites to release the intracellular domain, and (2) a chimeric antigen receptor (CAR) polypeptide that is operably linked to a promoter that is responsive to the transcriptional activator of the released intracellular domain of the P-selectin-specific chimeric receptor polypeptide. Also disclosed herein are methods of using MEAT cells of the present disclosure to treat cancer and mitigate toxicities associated with CAR T cell therapy.

In one aspect, the present disclosure provides an engineered T cell comprising a nucleic acid encoding a P-selectin-specific chimeric receptor polypeptide comprising an extracellular domain that specifically binds P-selectin; a heterologous receptor polypeptide comprising one or more ligand-inducible proteolytic cleavage sites; and an intracellular domain comprising a transcriptional activator, wherein binding of the extracellular domain to P-selectin induces cleavage of the heterologous receptor polypeptide at the one or more ligand-inducible proteolytic cleavage sites to release the intracellular domain, and a nucleic acid encoding a chimeric antigen receptor (CAR) polypeptide that is operably linked to a promoter that is responsive to the transcriptional activator of the released intracellular domain of the P-selectin-specific chimeric receptor polypeptide. The extracellular domain of the P-selectin-specific chimeric receptor polypeptide may comprise an anti-P-selectin antigen binding fragment or a PSGL-1 polypeptide. In certain embodiments, the anti-P-selectin antigen binding fragment is a Fab, F(ab′)₂, or a scFv. In other embodiments, the PSGL-1 polypeptide comprises an amino acid sequence having at least 90%, at least 95%, at least 98%, at least 99%, or 100%, amino acid sequence identity to SEQ ID NO: 92. Additionally or alternatively, in certain embodiments, the nucleic acid encoding the P-selectin-specific chimeric receptor polypeptide and/or the nucleic acid encoding the CAR polypeptide comprises a signal peptide sequence. The T cell may be a CD4+ T cell or a CD8+ T cell. In certain embodiments, the T cell is derived from an autologous donor or an allogenic donor.

Additionally or alternatively, in some embodiments of the engineered T cell described herein, the extracellular domain of the P-selectin specific chimeric receptor polypeptide comprises a heavy chain variable (V_{H}) region and a light chain variable (V_{L}) region, wherein the V_{H} region comprises a V_{H} CDR1 sequence comprising SYDIN (SEQ ID NO: 63), a V_{H} CDR2 sequence comprising WIYPGDGSIKYNEKFKG (SEQ ID NO: 64), and a V_{H} CDR3 sequence comprising RGEYGNYEGAMDY (SEQ ID NO: 65); and the V_{L} region comprises a V_{L} CDR1 sequence comprising KASQSVDYDGHSYMN (SEQ ID NO: 66), a V_{L} CDR2 sequence comprising AASNLES (SEQ ID NO: 67), and a V_{L} CDR3 sequence comprising QQSDENPLT (SEQ ID NO: 68); or the V_{H} region comprises a V_{H} CDR1 sequence comprising GYTFTSY (SEQ ID NO: 69), a V_{H} CDR2 sequence comprising DPYYGG (SEQ ID NO: 70), and a V_{H} CDR3 sequence comprising WDGYYGGFSY (SEQ ID NO: 71); and the V_{L} region comprises a V_{L} CDR1 sequence comprising RASSNVKYMY (SEQ ID NO: 72), a V_{L} CDR2 sequence comprising YTSNLAS (SEQ ID NO: 73), and a V_{L} CDR3 sequence comprising QQFTSSPYT (SEQ ID NO: 74); or the V_{H} region comprises a V_{H} CDR1 sequence comprising GYSITSDY (SEQ ID NO: 75), a V_{H} CDR2 sequence comprising SSGR (SEQ ID NO: 76), and a V_{H} CDR3 sequence comprising HYGNYEGYYYAMDY (SEQ ID NO: 77); and/or the V_{L} region comprises a V_{L} CDR1 sequence comprising ITSTGIDDDMN (SEQ ID NO: 78), a V_{L} CDR2 sequence comprising EGNVLRP (SEQ ID NO: 79), and a V_{L} CDR3 sequence comprising LQTDNLPLT (SEQ ID NO: 80).

Additionally or alternatively, in some embodiments, the extracellular domain of the P-selectin specific chimeric receptor polypeptide comprises a V_{H} amino acid sequence selected from the group consisting of; SEQ ID NO: 81, SEQ ID NO: 83, and SEQ ID NO: 85; and a V_{L} amino acid sequence selected from the group consisting of; SEQ ID NO: 82; SEQ ID NO: 84; and SEQ ID NO: 86. The chimeric receptor polypeptide may be expressed on the surface of the engineered T cell. In some embodiments, the heterologous receptor polypeptide comprises a Notch receptor polypeptide. The Notch receptor polypeptide may comprise an S2 proteolytic cleavage site and an S3 proteolytic cleavage site. In some embodiments, the Notch receptor polypeptide comprises the amino acid sequence of SEQ ID NO: 42 or SEQ ID NO: 43.

In any of the preceding embodiments, the transcriptional activator comprises Gal4-VP16. Additionally or alternatively, in some embodiments, the nucleic acid encoding the P-selectin-specific chimeric receptor polypeptide is operably linked to a promoter, such as a constitutive promoter or a conditional promoter.

In any and all embodiments of the engineered T cells disclosed herein, the CAR polypeptide comprises (i) an extracellular antigen binding domain; (ii) a transmembrane domain; and (iii) an intracellular domain. The extracellular antigen binding domain may bind to a target antigen, such as a tumor antigen. Examples of target antigens include, but are not limited to CD19, GD2, 5T4, alpha 5pi-integrin, 707-AP, A33, AFP, ART-4, B7H4, BAGE, Bcl-2, β-catenin, Bcr-abl, MN/C IX antibody, CA125, CA19-9, CAMEL, CAP-1, CASP-8, CD4, CD5, CD20, CD21, CD22, CD25, CDC27/m, CD33, CD37, CD45, CD52, CD56, CD80, CD123, CDK4/m, CEA, c-Met, CS-1, CT, Cyp-B, cyclin B1, DAGE, DAM, EBNA, EGFR, ErbB3, ELF2M, EMMPRIN, EpCam, ephrinB2, estrogen receptor, ETV6-AML1, FAP, ferritin, folate-binding protein, GAGE, G250, GM1, GM2, GnT-V, gp75, gp100 (Pmel 17), HAGE, HER-2/neu, HLA-A*0201-R170I, HPV E6, HPV E7, Ki-67, HSP70-2M, HST-2, hTERT (or hTRT), iCE, IGF-1R, IL-2R, IL-5, KIAA0205, LAGE, LDLR/FUT, LRP, MAGE, MART, MART-1/melan-A, MART-2/Ski, MC1R, mesothelin, MUC, MUM-1-B, myc, MUM-2, MUM-3, NA88-A, NYESO-1, NY-Eso-B, p53, proteinase-3, p190 minor ber-abl, Pml/RARa, PRAME, progesterone receptor, PSA, PSM, PSMA, ras, RAGE, RU1 or RU2, RORI, SART-1 or SART-3, survivin, TEL/AML1, TGFp, TPI/m, TRP-1, TRP-2, TRP-2/INT2, tenascin, TSTA tyrosinase, VEGF, and WT1.

Additionally or alternatively, in some embodiments, the extracellular antigen binding domain comprises a single chain variable fragment (scFv). The extracellular antigen binding domain may comprise a GD2 scFv having at least 80%, 85%>, 90%, 95%, 96%, 97%, 98%, or 99% sequence identity to SEQ ID NO: 3. In some embodiments, the extracellular antigen binding domain comprises a GD2 scFv of SEQ ID NO: 3. Additionally or alternatively, in certain embodiments, the transmembrane domain comprises a CD8 transmembrane domain. Additionally or alternatively, in some embodiments, the intracellular domain of the CAR polypeptide comprises one or more costimulatory domains. Examples of the one or more costimulatory domains include a CD28 costimulatory domain, a CD3ζ chain, a 4-1BBL costimulatory domain, or any combination thereof.

In one aspect, the present disclosure provides a method for treating cancer in a subject in need thereof comprising administering an effective amount of any and all embodiments of the engineered T cells described herein. The engineered T cells may be administered intravenously, intraperitoneally, subcutaneously, intramuscularly, or intratumorally. In some embodiments, the cancer is a carcinoma, sarcoma, a melanoma, or a hematopoietic cancer. Examples of cancer include, but are not limited to, adrenal cancers, bladder cancers, blood cancers, bone cancers, brain cancers, breast cancers, carcinoma, cervical cancers, colon cancers, colorectal cancers, corpus uterine cancers, ear, nose and throat (ENT) cancers, endometrial cancers, esophageal cancers, gastrointestinal cancers, head and neck cancers, Hodgkin's disease, intestinal cancers, kidney cancers, larynx cancers, leukemias, liver cancers, lymph node cancers, lymphomas, lung cancers, melanomas, mesothelioma, myelomas, nasopharynx cancers, neuroblastomas, non-Hodgkin's lymphoma, oral cancers, ovarian cancers, pancreatic cancers, penile cancers, pharynx cancers, prostate cancers, rectal cancers, sarcoma, seminomas, skin cancers, stomach cancers, teratomas, testicular cancers, thyroid cancers, uterine cancers, vaginal cancers, vascular tumors, and metastases thereof.

In some embodiments, the methods of the present technology further comprise administering an additional cancer therapy. Examples of additional cancer therapy include, but are not limited to chemotherapy, radiation therapy, immunotherapy, monoclonal antibodies, anticancer nucleic acids or proteins, anti-cancer viruses or microorganisms, and any combinations thereof. Additionally or alternatively, in some embodiments, the methods disclosed herein further comprise administering a cytokine to the subject. The cytokine may be administered prior to, during, or subsequent to administration of the one or more engineered T cells. Examples of cytokines include, but are not limited to, interferon a, interferon β, interferon γ, complement C5a, IL-2, TNF alpha, CD40L, IL12, IL-23, IL15, IL17, CCL1, CCL11, CCL12, CCL13, CCL14-1, CCL14-2, CCL14-3, CCL15-1, CCL15-2, CCL16, CCL17, CCL18, CCL19, CCL19, CCL2, CCL20, CCL21, CCL22, CCL23-1, CCL23-2, CCL24, CCL25-1, CCL25-2, CCL26, CCL27, CCL28, CCL3, CCL3L1, CCL4, CCL4L1, CCL5, CCL6, CCL7, CCL8, CCL9, CCR10, CCR2, CCR5, CCR6, CCR7, CCR8, CCRL1, CCRL2, CX3CL1, CX3CR, CXCL1, CXCL10, CXCL11, CXCL12, CXCL13, CXCL14, CXCL15, CXCL16, CXCL2, CXCL3, CXCL4, CXCL5, CXCL6, CXCL7, CXCL8, CXCL9, CXCL9, CXCR1, CXCR2, CXCR4, CXCR5, CXCR6, CXCR7 and XCL2.

In another aspect, the present disclosure provides a method for mitigating toxicities associated with CAR T cell therapy in a subject in need thereof comprising administering an effective amount of any and all embodiments of the engineered T cells described herein.

In yet another aspect, the present disclosure provides a vector comprising a nucleic acid encoding a P-selectin-specific chimeric receptor polypeptide comprising an extracellular domain that specifically binds P-selectin; a heterologous receptor polypeptide comprising one or more ligand-inducible proteolytic cleavage sites; and an intracellular domain comprising a transcriptional activator, wherein binding of the extracellular domain to P-selectin induces cleavage of the heterologous receptor polypeptide at the one or more ligand-inducible proteolytic cleavage sites to release the intracellular domain, and a nucleic acid encoding a chimeric antigen receptor (CAR) polypeptide that is operably linked to a promoter that is responsive to the transcriptional activator of the released intracellular domain of the P-selectin-specific chimeric receptor polypeptide. In some embodiments, the vector is a viral vector, a retroviral vector or a plasmid.

Additionally or alternatively, in some embodiments of the vector, the nucleic acid encoding the P-selectin-specific chimeric receptor polypeptide is operably linked to a promoter. The promoter may be a constitutive promoter or a conditional promoter.

Additionally or alternatively, in certain embodiments of the vector, the extracellular domain of the P-selectin-specific chimeric receptor polypeptide comprises an anti-P-selectin antigen binding fragment or a PSGL-1 polypeptide. In certain embodiments, the PSGL-1 polypeptide comprises an amino acid sequence having at least 90%, at least 95%, at least 98%, at least 99%, or 100%, amino acid sequence identity to SEQ ID NO: 92. In some embodiments, the extracellular domain of the P-selectin specific chimeric receptor polypeptide comprises a heavy chain variable (V_{H}) region and a light chain variable (V_{L}) region, wherein the V_{H} region comprises a V_{H} CDR1 sequence comprising SYDIN (SEQ ID NO: 63), a V_{H} CDR2 sequence comprising WIYPGDGSIKYNEKFKG (SEQ ID NO: 64), and a V_{H} CDR3 sequence comprising RGEYGNYEGAMDY (SEQ ID NO: 65); and the V_{L} region comprises a V_{L} CDR1 sequence comprising KASQSVDYDGHSYMN (SEQ ID NO: 66), a V_{L} CDR2 sequence comprising AASNLES (SEQ ID NO: 67), and a V_{L} CDR3 sequence comprising QQSDENPLT (SEQ ID NO: 68); or the V_{H} region comprises a V_{H} CDR1 sequence comprising GYTFTSY (SEQ ID NO: 69), a V_{H} CDR2 sequence comprising DPYYGG (SEQ ID NO: 70), and a V_{H} CDR3 sequence comprising WDGYYGGFSY (SEQ ID NO: 71); and the V_{L} region comprises a V_{L} CDR1 sequence comprising RASSNVKYMY (SEQ ID NO: 72), a V_{L} CDR2 sequence comprising YTSNLAS (SEQ ID NO: 73), and a V_{L} CDR3 sequence comprising QQFTSSPYT (SEQ ID NO: 74); or the V_{H} region comprises a V_{H} CDR1 sequence comprising GYSITSDY (SEQ ID NO: 75), a V_{H} CDR2 sequence comprising SSGR (SEQ ID NO: 76), and a V_{H} CDR3 sequence comprising HYGNYEGYYYAMDY (SEQ ID NO: 77); and/or the V_{L} region comprises a V_{L} CDR1 sequence comprising ITSTGIDDDMN (SEQ ID NO: 78), a V_{L} CDR2 sequence comprising EGNVLRP (SEQ ID NO: 79), and a V_{L} CDR3 sequence comprising LQTDNLPLT (SEQ ID NO: 80). In certain embodiments, the extracellular domain of the P-selectin specific chimeric receptor polypeptide comprises a V_{H} amino acid sequence selected from the group consisting of; SEQ ID NO: 81, SEQ ID NO: 83, and SEQ ID NO: 85; and a V_{L} amino acid sequence selected from the group consisting of; SEQ ID NO: 82; SEQ ID NO: 84; and SEQ ID NO: 86.

Additionally or alternatively, in some embodiments, the heterologous receptor polypeptide comprises a Notch receptor polypeptide. The Notch receptor polypeptide may comprise an S2 proteolytic cleavage site and an S3 proteolytic cleavage site. In some embodiments, the Notch receptor polypeptide comprises the amino acid sequence of SEQ ID NO: 42 or SEQ ID NO: 43. Additionally or alternatively, in some embodiments, the transcriptional activator comprises Gal4-VP16. In certain embodiments, the nucleic acid encoding the P-selectin-specific chimeric receptor polypeptide is operably linked to a promoter. The promoter may be a constitutive promoter or a conditional promoter.

Additionally or alternatively, in certain embodiments, the CAR polypeptide comprises (i) an extracellular antigen binding domain; (ii) a transmembrane domain; and (iii) an intracellular domain. The extracellular antigen binding domain may comprise a single chain variable fragment (scFv). In some embodiments, the extracellular antigen binding domain binds to a target antigen. In certain embodiments, the target antigen is a tumor antigen. In any of the foregoing embodiments, the extracellular antigen binding domain comprises a GD2 scFv of SEQ ID NO: 3.

In one aspect, the present disclosure provides a method for preparing T cells for cancer therapy, comprising isolating T cells from a donor subject, and transducing the T cells with any and all embodiments of the vector described herein. In another aspect, the present disclosure provides a method of treatment comprising isolating T cells from a donor subject, transducing the T cells with any and all embodiments of the vector described herein, and administering the transduced T cells to a recipient subject. The donor subject and the recipient subject may be the same or different. In certain embodiments, the T cell is a CD4+ T cell or a CD8+ T cell.

### BRIEF DESCRIPTION OF THE DRAWINGS

FIG. 1A: Design of lentiviral GD2 CAR construct.

FIG. 1B: Primary human T-cells were transduced with GD2 CAR and engrafted i.v. into NSG mice. Mouse weights were measured until weight dropped below 20% of starting value.

FIG. 1C: Quantification of NSG mouse weight treated with primary human GD2 CAR T-cells i.v. with varying doses.

FIG. 1D: Kaplan-Meier graph depicting survival of NSG mice treated with primary human GD2 CAR T-cells i.v. with varying doses.

FIG. 1E: Quantification of NSG mouse grip strength treated with primary human GD2 CAR T-cells i.v. with varying doses.

FIG. 1F: Representative images of CD8, CD4, and CD45 histological staining of NSG mouse brains treated with primary human GD2 CAR T-cells i.v, and a merged stain thereof with left-hand panels showing a zoom of the white box ROI. The bar graph shows a comparative analysis of CD45, CD4, and CD8 positive brain infiltrating populations of primary human GD2 CAR T-cells.

FIG. 1G: Comparative analysis of CD4 and CD8 positive brain infiltrating populations of primary human GD2 CAR T-cells. Raw T cell numbers in the brain in 3 different mice are displayed 3 ways.

FIG. 1H: Time course of GD2 CAR T cell expansion via bioluminescence imaging (BLI) of AkaLuc expressing primary T cells (Cntrl=uninjected control). All mice are in indicated order as in top left panel.

FIG. 1I: Ex vivo imaging of mouse brains at day 14 endpoint by BLI.

FIG. 1J: Quantification of brain-specific CAR T cell expansion.

FIG. 2A: SynNotch receptors are modular and enable selective expression of toxic CARs. The MEAT^{(SELP_GD2 CAR)} system contains an anti-(human or mouse) P-selectin SynNotch receptor, which upon engagement to its ligand, induces expression of GD2 CAR.

FIG. 2B: Lentiviral vector design of MEAT^{(SELP_GD2 CAR)} system.

FIG. 2C: Jurkat cells were transduced with human or mouse MEAT^{(SELP_GD2 CAR)} system. Jurkat^{(hSELP_GD2 CAR)} cells were co-cultured with soluble or plate bound recombinant human P-selectin for 24 hrs and Jurkat^{(mSELP_GD2 CAR)} cells were co-cultured with plate bound recombinant mouse P-selectin for 24 hrs. GD2 CAR expression was assayed through flow cytometric analysis using a fluorescently labeled anti-idiotype antibody reactive to the GD2 CAR.

FIG. 2D: Jurkat^{(hSELP_GD2 CAR)} cells were co-cultured with plate bound recombinant human P-selectin for 24 hrs. Jurkat^{(SELP_GD2 CAR)} cells were transferred to P-selectin free wells and GD2 CAR expression was assayed over 50 hrs through flow cytometric analysis using a fluorescently labeled anti-idiotype antibody reactive to the GD2 CAR.

FIG. 2E: Activation of Jurkat^{(SELP_GD2 CAR)} cells co-cultured for 24 hrs with K562 cells expressing the actuation ligand (hSELP) and the GD2 CAR target ligand (GD2). Normalized Median Florescent Intensity was calculated using data from flow cytometric analysis of fluorescently labeled antibody to the T-cell activation marker CD69.

FIG. 2F: Activation of human^{(SELP_GD2 CAR)} T-cells co-cultured for 24 hrs with K562 cells expressing the single the ligand (hSELP) or dual ligands (hSELP and GD2). Plotted is Median Florescent Intensity from flow cytometric analysis of fluorescently labeled antibody to the T-cell activation marker CD69.

FIG. 2G: Cytotoxcity assay of K562 cells expressing hSELP, GD2, or both co-cultured with Human^{(SELP_GD2 CAR)} T-cells for 48 hrs. Cytotoxicity was assayed through flow cytometric analysis using a fluorescently labeled antibodies reactive to K562 cells.

FIG. 3A: Primary human T-cells were transduced with GD2 CAR or MEAT^{(hSELP_GD2 CAR)} and engrafted i.v. into NSG mice.

FIG. 3B: Quantification of weight of NSG mice engrafted with GD2 CAR or human^{(SELP_GD2 CAR)} T-cells. Mouse weights were measured until weight dropped below 20% of starting value.

FIG. 3C: Primary human T-cells were transduced with GD2 CAR or MEAT^{(hsELP_GD2 CAR)} and engrafted i.v. into NSG mice. Mice were sacrificed at predetermined time points and tissues were collected for histological staining and flow cytometric analysis (FIGS. 3D-3F).

FIG. 3D: Representative images of CD45+ histological staining of mouse spleens post engraftment of T-cells transduced with GD2 CAR or human^{(hSELP_GD2 CAR)} Splenic weight time course.

FIG. 3E: T-cell engraftment of spleen and GD2 CAR expression assayed through flow cytometric analysis using a fluorescently labeled anti-idiotype antibody reactive to the GD2 CAR.

FIG. 3F: Representative images of CD45+ histological staining of mouse brains post engraftment of T-cells transduced with GD2 CAR or human^{(hSELP_GD2 CAR)} and T-cell infiltration time course.

FIG. 3G: Day 14 endpoint flow cytometric GD2 CAR expression assay of T-cells within mouse brains.

FIG. 4A: Schema of in vivo experiment with Anti-GD2 CAR T cells in mice with GD2 positive tumors.

FIG. 4B: Weight loss in mice that receive GD2 CAR T cells is far more severe than in MEAT cells (hSELP) even at 4× higher doses.

FIG. 4C: Antitumor effects of MEAT cells are comparable to anti-GD2 CAR T cells without negatively impacting survival.

FIG. 4D: MEAT T cells enter the brain with far less numbers as compared to anti-GD2 CAR T cells by immunofluorescence of brain.

FIG. 4E: Flow cytometry for CAR T cells in the Spleen.

FIG. 4F: Flow cytometry for CAR T cells in the brain.

FIGS. 5A-5C demonstrate that a MEAT-cell requires two target cells for it to become selectively cytotoxic: a normal “actuator” cell in the tumor microenvironment, and a target cancer cell residing in that tumor microenvironment. If these two conditions are met, the effector cell (MEAT-cell) will kill the target cancer cell. The MEAT-cells will have genetically encoded actuation elements and response elements (FIG. 5A). The actuation element will express a receptor that binds to a cognate ligand present on actuator cells (FIG. 5B). The response element will encode a CAR molecule that is turned on following actuation (FIG. 5C). Therefore, IF a MEAT-cell engages with actuator cells in the metastatic tissue, it will express its CAR molecule, and THEN kill target cells; creating a “IF A” “THEN B” logic decision for the cell. In contrast, if the MEAT-cell is not actuated (not expressing the CAR molecule) and encounters the CAR antigen in a different environment, for example on a healthy, non-cancerous tissue cell, it will not kill.

FIG. 6A: A schematic for assessing the ability of the Jurkat^{αGFP:CD19CAR} cells to actuate by culturing them on plates coated with anti-MYC antibodies—where interaction of the cells with the anti-Myc antibody replicates engagement of the synNotch receptor. Actuation was measured using either a TagBFP reporter gene and/or with a fluorescently labeled anti-idiotype antibody to the anti-CD19 CAR.

FIG. 6B: Time course of CAR expression (red) and BFB marker (blue) in MEAT cells after actuation by SynNotch.

FIG. 6C: Time course of CD69 cell marker for activation with CAR expression (pink) or controls (black has no target cells and dotted pink has no actuator molecule) in MEAT cells after actuation by SynNotch via anti-MYC antibody.

FIG. 6D: Cell activation as shown by CD69 expression in MEAT cells (pink) exposed to GFP target vs control cells without synNotch (blue). GFP is synNotch actuator; CD19 is CAR target.

FIG. 6E: Time course decay of CAR expression (red) and BFB marker (blue) in MEAT cells after removal of actuation by SynNotch.

FIG. 6F: Killing by MEAT cells (pink) vs control cells without both (white) or one of each of the necessary elements. (green and red).

FIG. 7A: A schematic for assessing the MEAT-cells' ability to actuate and activate in the presence of actuator and target cells. Jurkat^{αGFP:CD19CAR} MEAT cells are not actuated or activated by K562^{CD19} cells.

FIGS. 7B-7C: MEAT cells in presence of actuator targets and CD19 CAR Target cells at 1:1:1, kill the CD19 positive targets (pink) and not the actuator cells (green).

FIG. 8: Map of full length vector “combo” and a shorter “barebones” vector for making MEAT cells.

FIGS. 9A-9B: Killing as measured by flow cytometry by MEAT cells mixed with actuator target K562-GFP cells and/or CAR target K562-CD19 cells. FIG. 9A: Control. FIG. 9B: MEAT cells mixed 1:1:1 with actuators and target cells.

FIG. 10: (Upper left) Vector map for GD2 CAR T cells. (Upper right) Schema for experimental design. (Lower panels) Clinical toxicity of the GD2 CAR T cells as measured by weight loss, loss of grip strength, and survival.

FIG. 11A: Actuation by P-selectin of the LH vector design in MEAT cells is dose dependent. Left: flow cytometry data. Upper right: Cytometry data quantified. Middle right: Vector map. Lower right: Dose response. Figure discloses SEQ ID NO: 1.

FIG. 11B: Actuation by P-selectin of the HL vector design in MEAT cells is dose dependent. Left: flow cytometry data. Upper right: Cytometry data quantified. Middle right: Vector map. Lower right: Dose response. Figure discloses SEQ ID NO: 1.

FIG. 11C: Left: MEAT cells (HL synNotch) actuate in response to P selectin on plate but not soluble P selectin. Right: Kinetics of MEAT (LH and HL synNotch) actuation decay over time to 50 hrs.

FIG. 11D: Recombinant plate bound hSELP actuation of Jurkat^{HL:BFP αCD19_CAR} and Jurkat^{LH:BFP_αCD19CAR}.

FIG. 12: (Top Left) Time course of mouse weight loss in constitutive GD2 CAR T cells versus P-selectin gated MEAT cells (HL_GD2 CAR). (Top Right & bottom): Endpoint at D14 T cell infiltration in the brain by flow cytometry and immunofluorescence of cells.

FIG. 13A: Three different Mouse P selectin-reactive MEAT cells responding to recombinant P selectin on plate as measured by flow cytometry for CAR expression.

FIG. 13B: Three different Mouse P selectin-reactive MEAT cells responding to Lenti-x cells expressing mouse P selectin as measured by flow cytometry for CAR expression.

FIG. 14: Mechanism of action of the MEAT cells of the present disclosure.

FIGS. 15A-15B: Anti-mSELP Fab Based CARs present on the surface of transduced cells and activate with mSELP. FIG. 15A: Anti-mSELP Fab35CAR, Fab35FCAR, Fab60CAR, Fab60FCAR surface expression on transduced Jurkat cells through flow cytometry using an anti-MYC tag antibody. FIG. 15B: Cell activation of Jurkat^{Fab35CAR, Fab35FCAR, Fab60CAR, Fab60FCAR} measured by surface expression of CD69 through flow cytometry.

FIGS. 16A-16C: Actuation of Fab SynNotches with mSELP and surface detection of Fab SynNotches receptors. FIG. 16A: Jurkat^{Fab35,Fab35F,Fab60,Fab60F:αCD19CAR} actuation when cultured on mSELP coated 96 well plate. FIG. 16B: Jurkat^{Fab35,ab35F,ab60,Fab60F:αCD19CAR} actuation when cocultured with Lenti-X^{mSELP} cells. FIG. 16C Jurkat^{Fab35,ab35F,ab60,Fab60F:CAR} synNotch surface expression through flow cytometry with an anti-mouse Fab antibody.

FIGS. 17A-17D: Selective killing of SK-N-Be(2)^{CD19} target cells by primary human T-cells^{HL:CD19CAR}. FIGS. 17A, 17C: Viability of SK-N-Be(2)^{CD19} cells cocultured with Lenti-X^{mSELP} actuator cells and either primary human T-cells^{HL:CD19CAR} or mock transduced T-cells. FIGS. 17B, 17D: Viability of SK-N-Be(2)^{CD19} cells cocultured with Lenti-X^{hSELP} actuator cells and either primary human T-cells^{HL:CD19CAR} or mock transduced T-cells.

FIGS. 18A-18F: P-selectin is an actuator target in the TME. FIG. 18A Representative immunofluorescence stain of human neuroblastoma patient tissue for P-selectin (red), CD31 (green) and their co-localization (orange). Scale bar 200 m. FIG. 18B Quantification of P-selectin/CD31 expression in human neuroblastoma and normal peripheral nerve tissue via tissue microarray (n=27 tumor, n=9 normal). FIG. 18C Tumor microenvironment actuated T (MEAT) cell system. MEAT cells contain an anti-human P-selectin synNotch, which induces expression of either CD19 or GD2 CAR upon engagement to P-selectin. FIG. 18D MEAT cell constructs (i) αhSELP synNotch→GD2 CAR barebones vector (ii) αhSELP synNotch→CD19 CAR reporter vector. FIG. 18E Transduction efficiency of αhSELP synNotch→GD2 CAR MEAT primary T cells. FIG. 18F Jurkat MEAT cell actuation by plate-bound recombinant human P-selectin. Data represented by violin plot are individual data points, and statistics were calculated using unpaired t test (FIG. 18B). *P≤0.05, **P≤0.01 and *** P≤0.001.1

FIGS. 19A-19J: MEAT cells are specific and cytotoxic. FIG. 19A Actuation of primary αhSELP synNotch→CD19 CAR MEAT cells by soluble and plate-bound recombinant human P-selectin. FIG. 19B On kinetics of CD19 CAR in αhSELP synNotch→CD19 CAR MEAT primary T cells incubated with recombinant human P-selectin. FIG. 19C Time course of antigenic-specific activation in αhSELP synNotch→GD2 CAR MEAT cells co-cultured with GD2+ target cells as measured by CD69 expression. FIG. 19D Live cell killing co-culture schematic of effector (E), actuator (A) and target (T) cells. Live cell killing of (FIG. 19E) αhSELP synNotch→CD19 CAR MEAT cells and (FIG. 19F) CD19 CAR T cells at varying effector cell concentrations. FIG. 19G Fluorescence imaging of actuator (green) and target (red) cells co-cultured with either synNotch CD19 CAR, CD19 CAR or Mock T cells (unlabeled) for 72 hours. Live cell killing of (FIG. 19H) αhSELP synNotch→GD2 CAR MEAT cells and (FIG. 19I) GD2 CART cells at varying actuator cell concentrations. Grey dotted line represents 50% killing of target cells. FIG. 19J Top five cytokines secreted after 72 h co-culture of effector cells with target cells expressing both actuator and antigen (P-selectin⁺ and CD19⁺) or only one present (P-selectin⁺ or CD19⁺) as assayed by Luminex 25-plex human cytokine panel. Data are shown as means±SEM and statistics were calculated using one-way ANOVA (FIG. 19A) or two-way ANOVA with Tukey's post hoc test (FIG. 19J).

FIGS. 20A-20I: MEAT cells are less differentiated and more metabolically fit. FIGS. 20A-20C Resting (unstimulated) CD19 CAR, synNotch or Mock T cells assayed for surface expression of (FIG. 20A) Tim3, Lag3, and PD1 and (FIG. 20B) memory phenotype by flow cytometry. FIG. 20C SK-N-BE(2) target cell killing of unstimulated effector cells (E:T of 1:1). FIGS. 20D-20J CAR T cells were chronically activated for 15 days by coculture with 20% actuator (P-selectin⁺) and 80% target (CD19⁺) cells and with fresh actuator and target cells added on days 4, 8 and 12. Chronically activated CAR T cell (FIG. 20D) Tim3, Lag3, and PD1 surface expression and (FIG. 20E) memory phenotype by flow cytometry. FIG. 20F SK-N-BE(2) target cell killing of chronically activated effector cells (E:T of 1:1). FIGS. 20G-20J Metabolic analysis of effector cells following 15-day stimulation as measured by Seahorse mitochondrial stress test. FIGS. 20G Oxygen consumption rate (OCR), (FIG. 20H) spare respiratory capacity (SRC), and (FIG. 20I) extracellular acidification rate (ECAR). Flow data are shown as mean±SEM (n=3 biological replicates). Cytotoxicity and Seahorse data are shown as mean±SEM (n=3 technical replicates, n=2 biological replicates). T_{N} (naïve), T_{SCM} (stem cell memory), T_{CM} (central memory), T_{TM} (transitional memory), and TEM (effector memory). Data are shown as means±SEM and statistics were calculated using one-way ANOVA with Dunnett's post hoc test (FIGS. 20A-20B), unpaired t test (FIGS. 20B, 20D, 20E) or one-way ANOVA with Tukey's post hoc test (FIG. 20H).

FIGS. 21A-21H: P-selectin gated GD2 CAR T cells ameliorate toxicity. FIG. 21A Experimental scheme to assess efficacy of MEAT cells in a solid tumor model of neuroblastoma. Mice were treated with 3×10⁶ constitutive GD2 CAR T cells, αhSELP synNotch→GD2 CAR MEAT cells or control Mock T cells 14 days post-tumor inoculation. Tumor growth kinetics from SK-N-BE(2) tumor-bearing mice with (FIG. 21B) or without (FIG. 21C) P-selectin. FIG. 21D Top: Representative immunofluorescence staining of CD8+ and CD4+ cells in murine brain at survival endpoint (>20% weight loss or >2000 mm³ tumor size). Scale bar 50 um. Bottom: Quantification of T cell infiltration. Weight change in SK-N-BE(2) tumor-bearing mice with (FIG. 21E) or without (FIG. 21F) P-selectin. Kaplan-Meier survival curves from SK-N-BE(2) tumor-bearing mice with (FIG. 21G) or without (FIG. 21H) P-selectin. Data are shown as means±SEM and statistics were calculated using unpaired t test (FIGS. 21B-21C), one-way ANOVA with Tukey's post hoc test (d) or Kaplan-Meier survival analysis (FIGS. 21G-21H). N=5 biological replicates per group.

FIGS. 22A-22H: P-selectin gated CD19 CAR T cells improve T cell persistence and anti-tumor efficacy. Mice were treated with 3×10⁶ constitutive CD19 CAR T cells, αhSELP synNotch→CD19 CAR MEAT cells or control Mock T cells 14 days post-tumor inoculation. Tumor growth kinetics from SK-N-Be(2) tumor-bearing mice with (FIG. 22A) or without (FIG. 22B) P-selectin. Kaplan-Meier survival curves from SK-N-Be(2) tumor-bearing mice with (FIG. 22C) or without (FIG. 22D) P-selectin (N=5 biological replicates per group). Ex vivo flow cytometric analysis of (FIG. 22E) spleen, lung and (FIG. 22F) tumor infiltrating T cells. FIG. 22G Mixed antigen model to assess biodistribution of MEAT cells in a trans system. FIG. 22H Ex vivo flow cytometric analysis of tumor infiltrating T cells 17 days post-treatment. Data are shown as means±SEM and statistics were calculated using unpaired t test (FIGS. 22A-22B, 22E-22F), Kaplan-Meier survival analysis (FIGS. 22C-22D) or one-way ANOVA with Tukey's post hoc test (FIG. 22H).

FIGS. 23A-23D: GD2 CAR T cell toxicity is not dependent on cyclophosphamide pretreatment or intravenous injection route. FIG. 23A Weight change and FIG. 23B CD8 infiltration at endpoint (Day 7). FIG. 23C Weight change and FIG. 23D CD8+ and CD4+ T cell infiltration at endpoint (Day 13).

FIGS. 24A-24B: GD2 CAR T cell expansion via bioluminescence imaging (BLI) of AkaLuc expressing primary T cells (FIG. 24A) Time course of GD2 CAR T cell expansion by different anatomical viewpoints and (FIG. 24B) Endpoint ex vivo imaging of various organs as shown in FIGS. 1H and 11, respectively.

FIGS. 25A-25D: Off-target CD19 CAR does not infiltrate the CNS or cause neurotoxicity (FIG. 25A) Weight loss (FIG. 25B) CD4 infiltration (FIG. 25C) CD8 infiltration (FIG. 25D) Representative immunofluorescence stain (CD8 green, CD4 red). Scale bar is 800 μm.

FIG. 26: Neuroblastoma tissue microarray 27 cases of neuroblastoma, 5 normal peripheral nerve.

FIGS. 27A-27B: Normal brain tissue does not express P-selectin. FIG. 27A Tissue microarray of normal cerebrum stained for human P-selectin via IHC (n=24 cases). FIG. 27B Quantification of P-selectin positive cells from FIG. 27A.

FIGS. 28A-28B: CD19 CAR and synNotch validation and off kinetics. FIG. 28A Transduction efficiency of CD19(4-1BBz) CAR and αP-selectin synNotch CD19 CAR in primary human T cells. FIG. 28B Off kinetics of CD19 CAR in primary human T cells after actuation with P-selectin for 24 hours and removed from stimulus at indicated timepoints; (left) representative histograms of CAR surface expression and (right) summary of two biological donors.

FIGS. 29A-29C: Antigenic-specific activation. Time course of antigenic-specific activation in αhSELP synNotch→CD19 CAR MEAT cells co-cultured with P-selectin+ (FIG. 29A) or P-selectin− (FIG. 29B) target cells, as measured by CD69 expression. FIG. 29C Time course of antigenic-specific activation in αhSELP synNotch→GD2 CAR MEAT cells co-cultured with GD2+ target cells, as measured by CD69 expression (donor replicate FIG. 19C).

FIGS. 30A-30F: SK-N-BE(2) cell line generation and cis killing synNotch CD19 CAR. FIG. 30A Phenotype of SK-N-BE(2) lines endogenously expressing GD2 and overexpressing CD19 and/or P-selectin. Cis killing of target cells co-cultured with synNotch CD19 CAR, constitutive CD19 CAR, Mock T cell control or Target cells alone. SK-N-Be(2) target cell conditions are P-selectin+/CD19+(FIG. 30B), P-selectin+/CD19− (FIG. 30C), P-selectin−/CD19+ (FIG. 30D), and P-selectin−/CD19− target cells (FIG. 30E). FIG. 30F Quantification of hours to 50% killing. Grey dotted line represents 50% killing of target cells.

FIGS. 31A-31E: T98G cell line generation and cis killing synNotch GD2 CAR. FIG. 31A Phenotype of T98G lines endogenously expressing GD2 and overexpressing P-selectin. Quantification of hours to 50% killing for cis (FIG. 31B) and trans (FIG. 31C) co-cultures. Cis (FIG. 31D) and trans (FIG. 31E) live cell killing of target cells co-cultured with synNotch GD2 CAR, constitutive GD2 CAR, Mock T cell control or Target cells alone. Target cells express both GD2 and P-selectin in cis killing. Actuator cells express P-selectin and target cells express GD2 in trans killing. Grey dotted line represents 50% killing of target cells.

FIGS. 32A-32C: T98G cell line cytotoxicity raw data and biological repeats actuator cell titration (FIG. 32A) Donor 1 (FIG. 32B) Donor 2 and (FIG. 32C) Donor 3. Actuator cells express P-selectin and target cells express GD2 in trans killing. Grey dotted line represents 50% killing of target cells.

FIGS. 33A-33B: Additional cytokines profiled 72 hours post-incubation with target cells. FIG. 33A synNotch CD19 CART cells were co-incubated with either i) 20% P-selectin+ 80% CD19+ ii) 20% P-selectin+ 80% WT cells or iii) 20% WT 80% CD19+target cells for 72 hours. FIG. 33B Additional 20 cytokines detected by Luminex panel (top five shown in FIG. 19J.)

FIGS. 34A-34B: αhSELP synNotch→GD2 CAR T cell pre-infusion phenotype. Representative (FIG. 34A) exhaustion and (FIG. 34B) memory profile of resting primary CAR T cells 7 days post-transduction.

FIGS. 35A-35D: Oxygen consumption rate as assayed by Seahorse mitochondrial stress test of effector cells co-incubated with neuroblastoma target cells for 48 hours; schematic of experimental design FIG. 35A. Oxygen consumption rate (FIG. 35B) spare respiratory capacity (FIG. 35C) as determined by maximal respiration minus basal respiration and ECAR (FIG. 35D).

FIGS. 36A-36B: SK-N-BE(2) solid tumor model graphic (FIG. 36A) SynNotch GD2 CAR T cells actuate and kill in P-selectin positive TME (FIG. 36B) SynNotch GD2 CAR T cells do not express CAR and do not kill in P-selectin negative TME.

FIGS. 37A-37B: SynNotch TILs persist longer in neuroblastoma tumors than constitutive CAR. FIG. 37A Experimental scheme to assess persistence of MEAT cells in a solid tumor model of neuroblastoma. FIG. 37B Representative immunofluorescence staining of tumor tissue 14 days post-T cells (red: CD4 T cells, green: CD8 T cells).

FIGS. 38A-38G: Biological repeat for cis killing in SK-N-BE(2) xenograft. FIG. 38A Experimental scheme to assess efficacy of MEAT cells in a solid tumor model of neuroblastoma. Mice were treated with 3×10⁶ constitutive CAR T cells, αhSELP synNotch→CAR MEAT cells or control Mock T cells 14 days post-tumor inoculation. Tumor growth kinetics from SK-N-BE(2) tumor-bearing mice treated with CD19 (FIGS. 38B, 38D) or GD2 (FIGS. 38E, 38G) CAR T cell or synNotch T cells with and without P-selectin. Weight change in SK-N-BE(2) tumor-bearing mice with CD19 (FIG. 38C) or GD2 (FIG. 38F) CAR T cell or synNotch T cells with and without P-selectin. Data are shown as mean±SEM (n=5 biological replicates).

FIGS. 39A-39D: P-selectin gated CD19 CAR T cell ex vivo flow. FIG. 39A Experimental scheme to assess efficacy of MEAT cells in a solid tumor model of neuroblastoma. FIG. 39B Tumor mass of mice at endpoint (day 20). FIG. 39C Representative dot plot of CD3+/CD45+ tumor infiltrating lymphocytes. FIG. 39D Backgating of αhSELP synNotch→CD19 CAR sample in FIG. 39B to demonstrate gating strategy. Data are shown as mean±SEM (n=4 biological replicates).

FIG. 40A-40C: Trans killing in mixed antigen SK-N-BE(2) xenograft. FIG. 40A Experimental scheme to assess efficacy of MEAT cells in a mixed antigen tumor model of neuroblastoma. We engrafted 2 million SK-N-BE(2) neuroblastoma cells in the flank of NSG mice with 20% of the total cells expressing P-selectin and 80% expressing CD19. A separate cohort of control mice was engrafted with 20% WT SK-N-BE(2) negative for P-selectin and 80% expressing CD19. Mice were treated with 5×10⁶ constitutive CAR T cells, αhSELP synNotch→CAR MEAT cells or PBS 14 days post-tumor inoculation. FIG. 40B Tumor composition in untreated mice 19 days post-tumor engraftment. FIG. 40C Representative contour plot of CD3/CD45+ TILs in each treatment group.

### DETAILED DESCRIPTION

It is to be appreciated that certain aspects, modes, embodiments, variations and features of the present methods are described below in various levels of detail in order to provide a substantial understanding of the present technology. It is to be understood that the present disclosure is not limited to particular uses, methods, reagents, compounds, compositions or biological systems, which can, of course, vary. It is also to be understood that the terminology used herein is for the purpose of describing particular embodiments only, and is not intended to be limiting.

Patient T-cells can be engineered to express synthetic proteins such as chimeric antigen receptors (CARs) to redirect them to kill cancer cells. Current clinically used CAR T-cells kill by recognizing one cancer antigen. That said, there are few known cancer-specific cell surface antigens and the repertoire of truly specific CAR targets is limited. Consequently, CAR T-cells designed to treat solid tumors target antigens that are overly expressed on cancer cells compared to normal tissues. Unfortunately, the high potency of T-cells reacting with the normal cells can result in severe toxicity such as in preclinical models of GD2-targeted CAR T-cells. Specifically, toxicities associated with GD2 CAR T therapy are attributable to infiltration of GD2 CAR T cells into the brain and reactivity to GD2+ brain cells, thereby causing neurotoxicity.

Without wishing to be bound by theory, it is believed that cancer cells in the microenvironment may have a different surface antigen pattern than the surrounding stroma. The microenvironment actuated T (MEAT) cells of the present disclosure mitigate GD2 CAR-induced neurotoxicity by gating expression of the GD2 CAR within the metastatic tumor microenvironment. The MEAT cells utilize a microenvironment “sensor” that recognizes the P-selectin on the surface of tumor endothelial cells in microenvironment to initiate the production of CARs reactive to tumor cells. As demonstrated in the Examples herein, the MEAT cells of the present disclosure eliminate GD2 tumors in the presence of SELP environment in vivo without inducing toxicity. Thus, the MEAT cells increase the repertoire of usable cancer target ligands by selectively expressing CARs only within tumor microenvironments, thereby preventing on-target, off-tumor toxicity.

### Definitions

Before describing the disclosed embodiments in detail, it is to be understood that the present disclosure is not limited to particular compositions or biological systems, which can, of course, vary. It is also to be understood that the terminology used herein is for the purpose of describing particular embodiments only and is not intended to be limiting.

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

It is understood that embodiments of the present application described herein include “consisting of” and/or “consisting essentially of” embodiments.

As used herein, reference to “not” a value or parameter generally means and describes “other than” a value or parameter. For example, the method is not used to treat cancer of type X means the method is used to treat cancer of types other than X.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the “administration” of an agent or drug to a subject includes any route of introducing or delivering to a subject a compound to perform its intended function. Administration can be carried out by any suitable route, including but not limited to, orally, intranasally, parenterally (intravenously, intramuscularly, intraperitoneally, or subcutaneously), rectally, intrathecally, intratumorally or topically. Administration includes self-administration and the administration by another. “Administration” of a cell or vector or other agent and compositions containing same can be performed in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician or in the case of animals, by the treating veterinarian. In some embodiments, administering or a grammatical variation thereof also refers to more than one doses with certain interval. In some embodiments, the interval is 1 day, 2 days, 3 days, 4 days, 5 days, 6 days, 1 week, 10 days, 2 weeks, 3 weeks, 1 month, 2 months, 3 months, 4 months, 5 months, 6 months, 1 year or longer. In some embodiments, one dose is repeated for once, twice, three times, four times, five times, six times, seven times, eight times, nine times, ten times or more. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include oral administration, intraperitoneal, infusion, nasal administration, inhalation, injection, and topical application. In some embodiments, the administration is an infusion (for example to peripheral blood of a subject) over a certain period of time, such as about 30 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 24 hours or longer.

“Activation,” as used herein in relation to T cells, refers to the state of a T cell that has been sufficiently stimulated to induce detectable cellular proliferation. Activation can also be associated with induced cytokine production, and detectable effector functions.

The term “amino acid” refers to naturally occurring and non-naturally occurring amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and pyrolysine and selenocysteine. Amino acid analogs refer to agents that have the same basic chemical structure as a naturally occurring amino acid, i.e., an α carbon that is bound to a hydrogen, a carboxyl group, an amino group, and an R group, such as, homoserine, norleucine, methionine sulfoxide, methionine methyl sulfonium. Such analogs have modified R groups (such as, norleucine) or modified peptide backbones, but retain the same basic chemical structure as a naturally occurring amino acid. In some embodiments, amino acids forming a polypeptide are in the D form. In some embodiments, the amino acids forming a polypeptide are in the L form. In some embodiments, a first plurality of amino acids forming a polypeptide are in the D form, and a second plurality of amino acids are in the L form.

Amino acids are referred to herein by either their commonly known three letter symbols or by the one-letter symbols recommended by the IUPAC-IUB Biochemical Nomenclature Commission. Nucleotides, likewise, are referred to by their commonly accepted single-letter code.

As used herein, the term “analog” refers to a structurally related polypeptide or nucleic acid molecule having the function of a reference polypeptide or nucleic acid molecule.

As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments that lack the Fc fragment of intact antibody, clear more rapidly from the circulation, and may have less non-specific tissue binding of an intact antibody (Wahl et al., J. Nucl. Med. 24:316-325 (1983)). Antibodies may comprise whole native antibodies, monoclonal antibodies, human antibodies, humanized antibodies, camelised antibodies, multispecific antibodies, bispecific antibodies, chimeric antibodies, Fab, Fab′, single chain V region fragments (scFv), single domain antibodies (e.g., nanobodies and single domain camelid antibodies), VNAR fragments, Bi-specific T-cell engager (BiTE) antibodies, minibodies, disulfide-linked Fvs (sdFv), and anti-idiotypic (anti-Id) antibodies, intrabodies, fusion polypeptides, unconventional antibodies and antigen binding fragments of any of the above. In particular, antibodies include immunoglobulin molecules and immunologically active fragments of immunoglobulin molecules, i.e., molecules that contain an antigen binding site. Immunoglobulin molecules can be of any type (e.g., IgG, IgE, IgM, IgD, IgA, and IgY), class (e.g., IgG1, IgG2, IgG3, IgG4, IgA1, and IgA2), or subclass.

In certain embodiments, an antibody is a glycoprotein comprising at least two heavy (H) chains and two light (L) chains inter-connected by disulfide bonds. Each heavy chain is comprised of a heavy chain variable region (abbreviated herein as V_{H}) and a heavy chain constant (C_{H}) region. The heavy chain constant region is comprised of three domains, C_{H}1, C_{H}2, and C_{H}3. Each light chain is comprised of a light chain variable region (abbreviated herein as V_{L}) and a light chain constant C_{L} region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Cl q) of the classical complement system.

As used herein interchangeably, the terms “antigen binding portion”, “antigen binding fragment”, or “antigen binding region” of an antibody, refer to the region or portion of an antibody that binds to the antigen and which confers antigen specificity to the antibody; fragments of antigen binding proteins, for example antibodies, include one or more fragments of an antibody that retain the ability to specifically bind to an antigen. It has been shown that the antigen binding function of an antibody can be performed by fragments of a full-length antibody. Examples of antigen binding portions encompassed within the term “antibody fragments” of an antibody include a Fab fragment, a monovalent fragment consisting of the V_{L}, V_{H}, C_{L} and C_{H}1 domains; a F(ab)₂ fragment, a bivalent fragment comprising two Fab fragments linked by a disulfide bridge at the hinge region; a Fd fragment consisting of the V_{H} and C_{H}1 domains; a Fv fragment consisting of the V_{L} and V_{H} domains of a single arm of an antibody; a dAb fragment (Ward et al., Nature 341: 544-546 (1989)), which consists of a V_{H} domain; and an isolated complementarity determining region (CDR). An “isolated antibody” or “isolated antigen binding protein” is one which has been identified and separated and/or recovered from a component of its natural environment. “Synthetic antibodies” or “recombinant antibodies” are generally generated using recombinant technology or using peptide synthetic techniques known to those of skill in the art.

Antibodies and antibody fragments can be wholly or partially derived from mammals (e.g., humans, non-human primates, goats, guinea pigs, hamsters, horses, mice, rats, rabbits and sheep) or non-mammalian antibody producing animals (e.g., chickens, ducks, geese, snakes, and urodele amphibians). The antibodies and antibody fragments can be produced in animals or produced outside of animals, such as from yeast or phage (e.g., as a single antibody or antibody fragment or as part of an antibody library).

“Fv” is the minimum antibody fragment which contains a complete antigen-recognition and antigen-binding site. This fragment consists of a dimer of one heavy- and one light-chain variable region domain in tight, non-covalent association. From the folding of these two domains emanate six hypervariable loops (3 loops each from the heavy and light chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules. These are known as single chain Fv (scFv); see e.g., Bird et al., Science 242:423-426 (1988); and Huston et al., Proc. Natl. Acad. Sci. 85: 5879-5883 (1988). These antibody fragments are obtained using conventional techniques known to those of ordinary skill in the art, and the fragments are screened for utility in the same manner as are intact antibodies.

As used herein, the term “single-chain variable fragment” or “scFv” is a fusion protein of the variable regions of the heavy (V_{H}) and light chains (V_{L}) of an immunoglobulin (e.g., mouse or human) covalently linked to form a V_{H}::V_{L} heterodimer. The heavy (V_{H}) and light chains (V_{L}) are either joined directly or joined by a peptide-encoding linker (e.g., about 10, 15, 20, 25 amino acids), which connects the N-terminus of the V_{H} with the C-terminus of the V_{L}, or the C-terminus of the V_{H} with the N-terminus of the V_{L}. The linker is usually rich in glycine for flexibility, as well as serine or threonine for solubility. The linker can link the heavy chain variable region and the light chain variable region of the extracellular antigen binding domain. In certain embodiments, the linker comprises amino acids having GGGGSGGGGSGGGGS (SEQ ID NO: 1). In certain embodiments, the nucleic acid sequence encoding the amino acid sequence of SEQ ID NO: 1 is ggcggcggcggatctggaggtggtggctcaggtggcggaggctcc (SEQ ID NO: 2).

Despite removal of the constant regions and the introduction of a linker, scFv proteins retain the specificity of the original immunoglobulin. Single chain Fv polypeptide antibodies can be expressed from a nucleic acid comprising V_{H}- and V_{L}-encoding sequences as described by Huston, et al. (Proc. Nat. Acad. Sci. USA, 85:5879-5883 (1988)). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hybridoma (Larchmt) 27(6):455-51 (2008); Peter et al., J Cachexia Sarcopenia Muscle (2012); Shieh et al., J Imunol 183(4):2277-85 (2009); Giomarelli et al., Thromb Haemost 97(6):955-63 (2007); Fife eta., J Clin Invst 116(8):2252-61 (2006); Brocks et al., Immunotechnology 3(3): 173-84 (1997); Moosmayer et al., Ther Immunol 2(10):31-40 (1995). Agonistic scFvs having stimulatory activity have been described (see, e.g., Peter et al., J Biol Chem 25278(38):36740-7 (2003); Xie et al., Nat Biotech 15(8):768-71 (1997); Ledbetter et al., Crit Rev Immunol 17(5-6):427-55 (1997); Ho et al., Bio Chim Biophys Acta 1638(3):257-66 (2003)).

As used herein, an “antigen” refers to a molecule to which an antibody can selectively bind. The target antigen may be a protein (e.g., an antigenic peptide), carbohydrate, nucleic acid, lipid, hapten, or other naturally occurring or synthetic compound. An antigen may also be administered to an animal subject to generate an immune response in the subject. The term “epitope” as used herein refers to the specific group of atoms or amino acids on an antigen to which an antibody or antibody moiety binds.

By “binding affinity” is meant the strength of the total noncovalent interactions between a single binding site of a molecule (e.g., an antibody) and its binding partner (e.g., an antigen). Without wishing to be bound by theory, affinity depends on the closeness of stereochemical fit between antibody combining sites and antigen determinants, on the size of the area of contact between them, and on the distribution of charged and hydrophobic groups. Affinity also includes the term “avidity,” which refers to the strength of the antigen-antibody bond after formation of reversible complexes (e.g., either monovalent or multivalent). Methods for calculating the affinity of an antibody for an antigen are known in the art, comprising use of binding experiments to calculate affinity. The affinity of a molecule X for its partner Y can generally be represented by the dissociation constant (K_{d}). A low-affinity complex contains an antibody that generally tends to dissociate readily from the antigen, whereas a high-affinity complex contains an antibody that generally tends to remain bound to the antigen for a longer duration. Antibody activity in functional assays (e.g., flow cytometry assay) is also reflective of antibody affinity. Antibodies and affinities can be phenotypically characterized and compared using functional assays (e.g., flow cytometry assay).

The terms “cancer” or “tumor” are used interchangeably and refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell. As used herein, the term “cancer” includes premalignant, as well as malignant cancers.

As used herein, the term “CDR” or “complementarity determining region” is intended to mean the non-contiguous antigen combining sites found within the variable region of both heavy and light chain polypeptides. These particular regions have been described by Kabat et al., J. Biol. Chem. 252:6609-6616 (1977); Kabat et al., U.S. Dept. of Health and Human Services, “Sequences of proteins of immunological interest” (1991); by Chothia et al., J. Mol. Biol. 196:901-917 (1987); and MacCallum et al., J. Mol. Biol. 262:732-745 (1996), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues which encompass the CDRs as defined by each of the above cited references are set forth below in Table A as a comparison.

TABLE A
CDR Definitions

| | Kabat¹ | Chothia² | MacCallum³ | IMGT⁴ | AHo⁵ |
|---|---|---|---|---|---|
| V_{H} CDR1 | 31-35 | 26-32 | 30-35 | 27-38 | 25-40 |
| V_{H} CDR2 | 50-65 | 53-55 | 47-58 | 56-65 | 58-77 |
| V_{H} CDR3 | 95-102 | 96-101 | 93-101 | 105-117 | 109-137 |
| V_{L} CDR1 | 24-34 | 26-32 | 30-36 | 27-38 | 25-40 |
| V_{L} CDR2 | 50-56 | 50-52 | 46-55 | 56-65 | 58-77 |
| V_{L} CDR3 | 89-97 | 91-96 | 89-96 | 105-117 | 109-137 |

¹Residue numbering follows the nomenclature of Kabat et al., J. Biol. Chem. 252: 6609-6616 (1977); Kabat et al., U.S. Dept. of Health and Human Services, “Sequences of proteins of immunological interest” (1991).
²Residue numbering follows the nomenclature of Chothia et al., J. Mol. Biol. 196: 901-917 (1987); Al-Lazikani B. et al., J. Mol. Biol., 273: 927-948 (1997).
³Residue numbering follows the nomenclature of MacCallum et al., J. Mol. Biol. 262: 732-745 (1996); Abhinandan and Martin, Mol. Immunol., 45: 3832-3839 (2008).
⁴Residue numbering follows the nomenclature of Lefranc M.P. et al., Dev. Comp. Immunol., 27: 55-77 (2003); and Honegger and Plückthun, J. Mol. Biol., 309: 657-670 (2001).
⁵Residue numbering follows the nomenclature of Honegger and Plückthun, J. Mol. Biol., 309: 657-670 (2001).

The term “chimeric antibodies” refer to antibodies in which a portion of the heavy and/or light chain is identical or homologous to corresponding sequences in antibodies derived from a particular species or belonging to a particular antibody class or subclass, while the remainder of the chain(s) is identical or homologous to corresponding sequences in antibodies derived from another species or belonging to another antibody class or subclass, as well as fragments of such antibodies, so long as they exhibit a biological activity of interest (e.g., binding to GD2, CD19 etc.) (see U.S. Pat. No. 4,816,567; and Morrison et al., Proc. Natl. Acad. Sci. USA, 81:6851-6855 (1984)).

The term “chimeric antigen receptor (CAR)”, as used herein, refers to an artificially constructed hybrid single-chain protein or single-chain polypeptide containing an extracellular target-binding (e.g., antigen-binding) domain, linked directly or indirectly to a transmembrane domain (“TM domain”, e.g., the transmembrane domain of a costimulatory molecule), which is in turn linked directly or indirectly to an intracellular signaling domain (ISD) comprising a primary immune cell signaling domain (e.g., one involved in T cell or NK cell activation). The extracellular target-binding domain can be a single-chain variable fragment derived from an antibody (scFv). In addition to scFvs, other single chain antigen binding domains can be used in CAR, e.g., tandem scFvs, single-domain antibody fragments (VHHS or sdAbs), single domain bispecific antibodies (BsAbs), intrabodies, nanobodies, immunokines in a single chain format, and Fab, Fab′, or (Fab′)₂ in single chain formats. The extracellular target-binding domain can be joined to the TM domain via a flexible hinge/spacer region. The intracellular signaling domain (ISD) comprises a primary signaling sequence, or primary immune cell signaling sequence, which can be from an antigen-dependent, TCR-associated T cell activation molecule, e.g., a portion of the intracellular domain of CD3ζ (TCRζ), FcRγ, FcRβ, CD3γ, CD3δ, CD3ε, CD5, CD79a, CD79b, or CD66d. The ISD can further comprise a costimulatory signaling sequence; e.g., a portion of the intracellular domain of an antigen-independent, costimulatory molecule such as CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds CD83, Dap10, or the like. Characteristics of CARs include their ability to redirect immune cell (e.g., T cell or NK cell) specificity and reactivity toward a selected target in either MHC-restricted (in cases of TCR-mimic antibodies) or non-MHC-restricted (in cases of antibodies against cell surface proteins) manners, exploiting the antigen-binding properties of monoclonal antibodies. The non-MHC-restricted antigen recognition gives immune cells (e.g., T cells or NK cells) expressing CARs the ability to recognize antigen independent of antigen processing, thus bypassing a major mechanism of tumor escape.

There are currently three generations of CARs. The “first generation” CARs are typically single-chain polypeptides composed of an scFv as the antigen-binding domain fused to a transmembrane domain fused to the cytoplasmic/intracellular domain, which comprises a primary immune cell signaling sequence such as the intracellular domain from the CD3ζ chain, which is the primary transmitter of signals from endogenous TCRs. The “first generation” CARs can provide de novo antigen recognition and cause activation of both CD4⁺ and CD8⁺ T cells through their CD3ζ chain signaling domain in a single fusion molecule, independent of HLA-mediated antigen presentation. The “second generation” CARs add intracellular domains from various costimulatory molecules (e.g., CD28, 4-1BB, ICOS, OX40) to the primary immune cell signaling sequence of the CAR to provide additional signals to the T cell. Thus, the “second generation” CARs comprise fragments that provide costimulation (e.g., CD28 or 4-IBB) and activation (e.g., CD3ζ). Preclinical studies have indicated that the “second generation” CARs can improve the antitumor activity of T cells.

The “third generation” CARs comprise those that provide multiple costimulation (e.g., CD28 and 4-1BB) and activation (e.g., CD3ζ). Examples of CAR T therapies are described, see, e.g., U.S. Pat. No. 10,221,245 describing CAR CTL019 which has an anti-CD33 extracellular target-binding domain, a transmembrane domain from CD8, a costimulatory domain from 4-1BB, and a primary signaling domain from CD3ζ, as well as U.S. Pat. No. 9,855,298 which describes a CAR having an anti-CD33 extracellular target-binding domain, a costimulatory domain from CD28, and a primary signaling domain from CD3ζ.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed.

The term “diabodies” refers to small antibody fragments prepared by constructing scFv fragments typically with short linkers (such as about 5 to about 10 residues) between the V_{H} and V_{L} domains such that inter-chain but not intra-chain pairing of the V domains is achieved, resulting in a bivalent fragment, i.e., fragment having two antigen-binding sites. Bispecific diabodies are heterodimers of two “crossover” scFv fragments in which the V_{H} and V_{L} domains of the two antibodies are present on different polypeptide chains. Diabodies are described more fully in, for example, EP 404,097; WO 93/11161; and Hollinger et al., Proc. Natl. Acad. Sci. USA, 90:6444-6448 (1993).

As used herein, the term “effective amount” or “therapeutically effective amount” refers to a quantity of an agent sufficient to achieve a beneficial or desired clinical result upon treatment. In the context of therapeutic applications, the amount of a therapeutic agent administered to the subject can depend on the type and severity of the disease or condition and on the characteristics of the individual, such as general health, age, sex, body weight, effective concentration of the MEAT cells administered, and tolerance to drugs. It can also depend on the degree, severity, and type of disease. The skilled artisan will be able to determine appropriate dosages depending on these and other factors. An effective amount can be administered to a subject in one or more doses. In terms of treatment, an effective amount is an amount that is sufficient to palliate, ameliorate, stabilize, reverse or slow the progression of the disease, or otherwise reduce the pathological consequences of the disease. The effective amount is generally determined by the physician on a case-by-case basis and is within the skill of one in the art.

“Heterologous,” as used herein, means a nucleotide or polypeptide sequence that is not found in the native (e.g., naturally-occurring) nucleic acid or protein, respectively.

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a complementarity determining region (CDR) of the recipient are replaced by residues from a CDR of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies can comprise residues that are not found in the recipient antibody or in the donor antibody. These modifications are made to further refine antibody performance (e.g., affinity for the target antigen). In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

As used herein, the term “isolated,” “purified,” or “biologically pure” refers to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or polypeptide of the presently disclosed subject matter is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

As used herein, the “percent homology” between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm.

The percent homology between two amino acid sequences can be determined using the algorithm of E. Meyers and W. Miller (Comput. Appl. Biosci., 4: 1 1-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the amino acids sequences of the presently disclosed subject matter can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the specified sequences disclosed herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may, in some version, contain an intron(s).

The term “operably linked” refers to a functional linkage between two nucleic acid components wherein the components so described are in a relationship permitting them to function in their intended manner. Namely, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

As used herein, by “pharmaceutically acceptable” or “pharmacologically compatible” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

The terms “polypeptide,” “peptide,” and “protein” are used interchangeably herein to refer to a polymer of amino acid residues. The terms apply to naturally occurring amino acid polymers as well as amino acid polymers in which one or more amino acid residues are a non-naturally occurring amino acid, e.g., an amino acid analog. The terms encompass amino acid chains of any length, including full length proteins, wherein the amino acid residues are linked by covalent peptide bonds.

As used herein, the term “specifically binds” or “specifically binds to” or “specifically target” refers to a molecule (e.g., a polypeptide or fragment thereof) that recognizes and binds a molecule of interest (e.g., an antigen), but which does not substantially recognize and bind other molecules. The terms “specific binding,” “specifically binds to,” or is “specific for” a particular molecule (e.g., an antigen), as used herein, can be exhibited, for example, by a molecule having a K_{d} for the molecule to which it binds to of about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, or 10⁻¹² M.

As used herein, the terms “subject”, “patient”, or “individual” can be an individual organism, a vertebrate, a mammal, or a human. In some embodiments, the subject, patient or individual is a human.

The terms “substantially homologous” or “substantially identical” mean a polypeptide or nucleic acid molecule that exhibits at least 50% or greater homology or identity to a reference amino acid sequence (for example, any one of the amino acid sequences described herein) or nucleic acid sequence (for example, any one of the nucleic acid sequences described herein). For example, such a sequence is at least about 60%, about 65%, about 70%, about 75%, about 80%, about 85%, about 90%, about 95% or about 99% homologous or identical at the amino acid level or nucleic acid to the sequence used for comparison (e.g., a wild-type, or native, sequence). In some embodiments, a substantially homologous or substantially identical polypeptide contains one or more amino acid substitutions, insertions, or deletions relative to the sequence used for comparison. In some embodiments, a substantially homologous or substantially identical polypeptide contains one or more non-natural amino acids or amino acid analogs, including, D-amino acids and retroinverso amino, to replace homologous sequences.

Sequence homology or sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. In an exemplary approach to determining the degree of identity, a BLAST program may be used, with a probability score between e⁻³ and e⁻¹⁰⁰ indicating a closely related sequence.

Nucleic acid molecules useful in the presently disclosed subject matter include any nucleic acid molecule that encodes a polypeptide or a fragment thereof. Such nucleic acid molecules need not be 100% identical with an endogenous nucleic acid sequence, but will typically exhibit substantial identity. Polynucleotides having “substantial homology” or “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger, Methods Enzymol. 152:399 (1987); Kimmel, A. R. Methods Enzymol. 152:507 (1987)). For example, stringent salt concentration will ordinarily be less than about 750 mM NaCl and 75 mM trisodium citrate, less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% w/v formamide, or at least about 50% w/v formamide. Stringent temperature conditions will ordinarily include temperatures of at least about 30° C., at least about 37° C., or at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In certain embodiments, hybridization will occur at 30° C. in 750 mM NaCl, 75 mM trisodium citrate, and 1% w/v SDS. In certain embodiments, hybridization will occur at 37° C. in 500 mM NaCl, 50 mM trisodium citrate, 1% w/v SDS, 35% w/v formamide, and 100 g/ml denatured salmon sperm DNA (ssDNA). In certain embodiments, hybridization will occur at 42° C. in 250 mM NaCl, 25 mM trisodium citrate, 1% w/v SDS, 50% w/v formamide, and 200 μg ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will less than about 30 mM NaCl and 3 mM trisodium citrate, or less than about 15 mM NaCl and 1.5 mM trisodium citrate. Stringent temperature conditions for the wash steps will ordinarily include a temperature of at least about 25° C., at least about 42° C., or at least about 68° C. In certain embodiments, wash steps will occur at 25° C. in 30 mM NaCl, 3 mM trisodium citrate, and 0.1% w/v SDS. In certain embodiments, wash steps will occur at 42° C. in 15 mM NaCl, 1.5 mM trisodium citrate, and 0.1% w/v SDS. In certain embodiments, wash steps will occur at 68° C. in 15 mM NaCl, 1.5 mM trisodium citrate, and 0.1% w/v SDS. Additional variations on these conditions will be readily apparent to those skilled in the art. Hybridization techniques are well known to those skilled in the art and are described, for example, in Benton and Davis (Science 196: 180 (1977)); Grunstein and Rogness (Proc. Natl. Acad. Sci., USA 72:3961 (1975)); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. Therapeutic effects of treatment include, without limitation, inhibiting recurrence of disease, alleviation of symptoms, diminishment of any direct or indirect pathological consequences of the disease, preventing metastases, decreasing the rate of disease progression, amelioration or palliation of the disease state, and remission or improved prognosis. By “treating a cancer” is meant that the symptoms associated with the cancer are, e.g., alleviated, reduced, cured, or placed in a state of remission.

It is also to be appreciated that the various modes of treatment of diseases as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

A “vector” or “expression vector” is a replicon, such as plasmid, phage, virus, or cosmid, to which another DNA segment, i.e. an “insert”, may be attached so as to bring about the replication of the attached segment in a cell.

### P-Selectin-Specific Chimeric Receptor Polypeptides

The present disclosure provides a P-selectin-specific chimeric receptor polypeptide comprising: a) an extracellular domain that specifically binds P-selectin; b) a heterologous receptor polypeptide (e.g., a Notch receptor polypeptide) comprising one or more ligand-inducible proteolytic cleavage sites; and c) an intracellular domain comprising a transcriptional activator, wherein binding of the extracellular domain to P-selectin induces cleavage of the heterologous receptor polypeptide at the one or more ligand-inducible proteolytic cleavage sites to release the intracellular domain (e.g., a Notch receptor polypeptide).

Extracellular Domain. A P-selectin-specific chimeric receptor polypeptide of the present disclosure comprises an extracellular domain that specifically binds P-selectin. The extracellular domain comprises a P-selectin binding region that is heterologous to the receptor poly peptide (e.g., Notch receptor polypeptide). P-selectin is separate from (e.g. not covalently linked to) the P-selectin-specific chimeric receptor polypeptide comprising the extracellular domain that specifically binds P-selectin. In some embodiments, P-selectin is expressed on the surface of a cell. In certain embodiments, P-selectin is expressed on the surface of a tumor endothelial cell.

In some embodiments, the extracellular domain comprises an anti-P-selectin antigen binding fragment (e.g., scFv, Fab, (Fab′)₂). In certain embodiments, the extracellular domain comprises a PSGL-1 polypeptide.

In some embodiments, the extracellular domain of a P-selectin specific chimeric receptor polypeptide of the present disclosure comprises a heavy chain variable (V_{H}) region and a light chain variable (V_{L}) region, wherein the V_{H} region comprises a V_{H} CDR1 sequence comprising SYDIN (SEQ ID NO: 63), a V_{H} CDR2 sequence comprising WIYPGDGSIKYNEKFKG (SEQ ID NO: 64), and a V_{H} CDR3 sequence comprising RGEYGNYEGAMDY (SEQ ID NO: 65); and/or the V_{L} region comprises a V_{L} CDR1 sequence comprising KASQSVDYDGHSYMN (SEQ ID NO: 66), a V_{L} CDR2 sequence comprising AASNLES (SEQ ID NO: 67), and a V_{L} CDR3 sequence comprising QQSDENPLT (SEQ ID NO: 68).

In some embodiments, the extracellular domain of a P-selectin specific chimeric receptor polypeptide of the present disclosure comprises a heavy chain variable (V_{H}) region and a light chain variable (V_{L}) region, wherein the V_{H} region comprises a V_{H} CDR1 sequence comprising GYTFTSY (SEQ ID NO: 69), a V_{H} CDR2 sequence comprising DPYYGG (SEQ ID NO: 70), and a V_{H} CDR3 sequence comprising WDGYYGGFSY (SEQ ID NO: 71); and/or the V_{L} region comprises a V_{L} CDR1 sequence comprising RASSNVKYMY (SEQ ID NO: 72), a V_{L} CDR2 sequence comprising YTSNLAS (SEQ ID NO: 73), and a VLCDR3 sequence comprising QQFTSSPYT (SEQ ID NO: 74).

In some embodiments, the extracellular domain of a P-selectin specific chimeric receptor polypeptide of the present disclosure comprises a heavy chain variable (V_{H}) region and a light chain variable (V_{L}) region, wherein the V_{H} region comprises a V_{H} CDR1 sequence comprising GYSITSDY (SEQ ID NO: 75), a V_{H} CDR2 sequence comprising SSGR (SEQ ID NO: 76), and a V_{H} CDR3 sequence comprising HYGNYEGYYYAMDY (SEQ ID NO: 77); and/or the V_{L} region comprises a V_{L} CDR1 sequence comprising ITSTGIDDDMN (SEQ ID NO: 78), a V_{L} CDR2 sequence comprising EGNVLRP (SEQ ID NO: 79), and a V_{L} CDR3 sequence comprising LQTDNLPLT (SEQ ID NO: 80).

Additionally or alternatively, in some embodiments, the extracellular domain of a P-selectin specific chimeric receptor polypeptide of the present disclosure comprises a heavy chain variable (V_{H}) region and a light chain variable (V_{L}) region, wherein (a) the V_{H} comprises an amino acid sequence selected from the group consisting of:

(SEQ ID NO: 81)
QVQLVQSGAEVKKPGASVKVSCKVSGYTFTSYDINWVRQAPGKGL
EWMGWIYPGDGSIKYNEKFKGRVTMTVDKSTDTAYMELSSLRSED
TAVYYCARRGEYGNYEGAMDYWGQGTLVTVSS;
(SEQ ID NO: 83)
MAQVKLQQSGPELVKPGASVKMSCKASGYTFTSYVMHWVKQSNGK
SLEWIGTIDPYYGGTSYNQKFKGKATLTVDKSSTTAYIQLKSLTS
EDSAVYYCARWDGYYGGFSYWGQGTMVTVSS;
and
(SEQ ID NO: 85)
DVQLQESGPGLVKPSQSLSLTCTVTGYSITSDYAWNWIRQFPGNK
LEWMGYISSGRTSYNPSLKSRISITRDTSKNQFFLQLNSVTTEDT
ATYYCARHYGNYEGYYYAMDYWGQGTSVTVSS;

and/or (b) the V_{L} comprises an amino acid sequence selected from the group consisting of:

(SEQ ID NO: 82)
DIQMTQSPSSLSASVGDRVTITCKASQSVDYDGHSYMNWYQQKPG
KAPKLLIYAASNLESGVPSRFSGSGSGTDFTLTISSLQPEDFATY
YCQQSDENPLTFGGGTKVEIKR;
(SEQ ID NO: 84)
DIELTQSPAIMSATLGEKVTMSCRASSNVKYMYWYQQKSGASPKL
WIYYTSNLASGVPARFSGSGSGTSYSLTISSVEAEDAATYYCQQF
TSSPYTFGSGTKLEIKRAAAGAPVPYPDPLEPRAA;
and
(SEQ ID NO: 86)
ETTVTQSPASLSMAIGEKVTIRCITSTGIDDDMNWYQQKPGEPPE
LLISEGNVLRPGVPSRFSSSGYGTDFLFTIENILSEDVADYYCLQ
TDNLPLTFGSGTKLEIKR.

Additionally or alternatively, in some embodiments, the extracellular domain of a P-selectin specific chimeric receptor polypeptide of the present disclosure comprises a heavy chain variable (V_{H}) region amino acid sequence and a light chain variable (V_{L}) region amino acid sequence selected from the group consisting of SEQ ID NO: 81 and SEQ ID NO: 82, SEQ ID NO: 83 and SEQ ID NO: 84, and SEQ ID NO: 85 and SEQ ID NO: 86.

Exemplary scFv sequences of a P-selectin specific chimeric receptor polypeptide of the present disclosure that specifically bind P-selectin include, but are not limited to:

HL (Crizanlizumab) scFv Sequence:
(SEQ ID NO: 87)
QVQLVQSGAEVKKPGASVKVSCKVSGYTFTSYDINWVRQAPGKGLEWMGWIYP
GDGSIKYNEKFKGRVTMTVDKSTDTAYMELSSLRSEDTAVYYCARRGEYGNYE
GAMDYWGQGTLVTVSSGGGGSGGGGSGGGGSDIQMTQSPSSLSASVGDRVTITCK
ASQSVDYDGHSYMNWYQQKPGKAPKLLIYAASNLESGVPSRFSGSGSGTDFTLTIS
SLOPEDFATYYCQQSDENPLTFGGGTKVEIKR
LH (Crizanlizumab) scFV Sequence:
(SEQ ID NO: 88)
DIQMTQSPSSLSASVGDRVTITCKASQSVDYDGHSYMNWYQQKPGKAPKLLIYAA
SNLESGVPSRFSGSGSGTDFTLTISSLQPEDFATYYCQQSDENPLTFGGGTKVEIKR
GGGGSGGGGSGGGGSQVQLVQSGAEVKKPGASVKVSCKVSGYTFTSYDINWVRQ
APGKGLEWMGWIYPGDGSIKYNEKFKGRVTMTVDKSTDTAYMELSSLRSEDTA
VYYCARRGEYGNYEGAMDYWGQGTLVTVSS
10a scFV sequence:
(SEQ ID NO: 89)
MAQVKLQQSGPELVKPGASVKMSCKASGYTFTSYVMHWVKQSNGKSLEWIGTID
PYYGGTSYNQKFKGKATLTVDKSSTTAYIQLKSLTSEDSAVYYCARWDGYYGGF
SYWGQGTMVTVSSGGGGSGGGGSGGGGSDIELTQSPAIMSATLGEKVTMSCRASS
NVKYMYWYQQKSGASPKLWIYYTSNLASGVPARFSGSGSGTSYSLTISSVEAEDA
ATYYCQQFTSSPYTFGSGTKLEIKRAAAGAPVPYPDPLEPRAA
Rapid Novor P-selectin (VL-VH) derived from BioLegend
(SEQ ID NO: 90)
ETTVTQSPASLSMAIGEKVTIRCITSTGIDDDMNWYQQKPGEPPELLISE
GNVLRPGVPSRFSSSGYGTDFLFTIENILSEDVADYYCLQTDNLPLTFGSGTKLEIK
RGGGGSGGGGSGGGGSDVQLQESGPGLVKPSQSLSLTCTVTGYSITSDYAWNWIR
QFPGNKLEWMGYISSGRTSYNPSLKSRISITRDTSKNQFFLQLNSVTTEDTATYYCA
RHYGNYEGYYYAMDYWGQGTSVTVSS
(SEQ ID NO: 91)
DVQLQESGPGLVKPSQSLSLTCTVTGYSITSDYAWNWIRQFPGNKLEW
MGYISSGRTSYNPSLKSRISITRDTSKNQFFLQLNSVTTEDTATYYCARHYGNYEG
YYYAMDYWGQGTSVTVSSGGGGSGGGGSGGGGSETTVTQSPASLSMAIGEKVTIR
CITSTGIDDDMNWYQQKPGEPPELLISEGNVLRPGVPSRFSSSGYGTDFLFTIENILS
EDVADYYCLQTDNLPLTFGSGTKLEIKR

In some embodiments, the extracellular domain of a P-selectin specific chimeric receptor polypeptide of the present disclosure comprises an amino acid sequence having at least 75%, at least 80%, at least 85%, at least 90%, at least 95%, at least 98%, at least 99%, or 100%, amino acid sequence identity to any one of SEQ ID NOs: 87-91.

In some embodiments, the extracellular domain of a P-selectin specific chimeric receptor polypeptide of the present disclosure comprises a PSGL-1 polypeptide comprising the amino acid sequence of

(SEQ ID NO: 92)
MPLQLLLLLILLGPGNSLQLWDTWADEAEKALGPLLARDRRQATE
YEYLDYDFLPETEPPEMLRNSTDTTPLTGPGTPESTTVEPAARRS
TGLDAGGAVTELTTELANMGNLSTDSAAMEIQTTQPAATEAQTTQ
PVPTEAQTTPLAATEAQTTRLTATEAQTTPLAATEAQTTPPAATE
AQTTQPTGLEAQTTAPAAMEAQTTAPAAMEAQTTPPAAMEAQTTQ
TTAMEAQTTAPEATEAQTTQPTATEAQTTPLAAMEALSTEPSATE
ALSMEPTTKRGLFIPFSVSSVTHKGIPMAASNLSVNYPVGAPDHI
SVKQCLLAILILALVATIFFVCTVVLAVRLSRKGHMYPVRNYSPT
EMVCISSLLPDGGEGPSATANGGLSKAKSPGLTPEPREDREGDDL
TLHSFLP.

In some embodiments, the extracellular domain of a P-selectin specific chimeric receptor polypeptide of the present disclosure comprises an amino acid sequence having at least 75%, at least 80%, at least 85%, at least 90%, at least 95%, at least 98%, at least 99%, or 100%, amino acid sequence identity to SEQ ID NO: 92.

Heterologous Receptor Polypeptide. In some embodiments, the heterologous receptor polypeptide of the P-selectin-specific chimeric receptor polypeptide disclosed herein comprises a Notch receptor polypeptide. Additionally or alternatively, in some embodiments, the P-selectin-specific chimeric receptor polypeptide of the present disclosure comprises a Notch receptor polypeptide having a length of from 50 amino acids to 1000 amino acids and comprising one or more ligand-inducible proteolytic cleavage sites.

Mammalian Notch receptor polypeptide generally includes: a) an extracellular portion that includes: i) epidermal growth factor (EGF) repeats; ii) a ligand binding site; iii) three Lin-12 Notch repeats (LNR), designated LNR-A, LNR-B, and LNR-C; iv) two heterodimerization domains (HD-N and HD-C); b) a transmembrane (TM) portion; and c) an intracellular portion that includes: i) a RAM domain; ii) ankyrin repeats; iii) a transcription activation domain; and iv) a PEST region. A Notch receptor polypeptide includes three proteolytic sites, termed S1, S2, and S3. S1, a furin cleavage site, is located between HD-N and HC-C; S2, an ADAM17 cleavage site, is located within HD-C; and S3, a gamma secretase cleavage site, is within the TM portion. A Notch receptor polypeptide mediates cell-to-cell communication, e.g. communication between contacting cells, in which one contacting cell is a “receiver” cell and the other contacting cell is a “sender” cell. Engagement of a Notch receptor polypeptide present on a receiving cell by a Delta polypeptide (“ligand”) present on a sending cell results in ligand-induced cleavage of the Notch receptor polypeptide, resulting in release of the intracellular portion of the receptor from the membrane into the cytoplasm. The released portion alters receiver cell behavior by functioning as a transcriptional regulator. Exemplary amino acid sequence of Notch receptor is provided below:

Neurogenic locus notch homolog protein 1 preproprotein [Homo sapiens],
NCBI Reference Sequence: NP_060087.3 (SEQ ID NO: 37)

| | |
|---|---|
| 1 | mppllapllc lallpalaar gprcsqpget clnggkceaa ngteacvcgg afvgprcqdp |
| 61 | npclstpckn agtchvvdrr gvadyacsca lgfsgplclt pldnacltnp crnggtcdll |
| 121 | tlteykcrcp pgwsgkscqq adpcasnpca nggqclpfea syichcppsf hgptcrqdvn |
| 181 | ecgqkpgler hggtchnevg syrcvcrath tgpncerpyv pcspspcqng gtcrptgdvt |
| 241 | hecaclpgft gqnceenidd cpgnnckngg acvdgvntyn crcppewtgq yctedvdecq |
| 301 | lmpnacqngg tchnthggyn cvcvngwtge dcseniddca saacfhgatc hdrvasfyce |
| 361 | cphgrtgllc hlndacisnp cnegsncdtn pvngkaictc psgytgpacs qdvdecslga |
| 421 | npcehagkci ntlgsfecqc lqgytgprce idvnecvsnp condatcldq igefqcicmp |
| 481 | gyegvhcevn tdecasspcl hngrcldkin efqcecptgf tghlcqydvd ecastpckng |
| 541 | akcldgpnty tcvctegytg thcevdidec dpdpchygsc kdgvatftcl crpgytghhc |
| 601 | etninecssq pcrhggtcqd rdnaylcfcl kgttgpncei nlddcasspc dsgtcldkid |
| 661 | gyecacepgy tgsmcninid ecagnpchng gtcedgingf torcpegyhd ptclsevnec |
| 721 | nsnpcvhgac rdslngykcd cdpgwsgtnc dinnnecesn pcvnggtckd mtsgyvctcr |
| 871 | egfsgpncqt ninecasnpc Inqgtciddv agykcncllp ytgatcevvl apcapspcrn |
| 841 | ggecrqsedy esfscvcptg wqgqtcevdi necvlspcrh gascqnthgg yrchcqagys |
| 901 | grncetdidd crpnpchngg sctdgintaf cdclpgfrgt fceedineca sdpcrnganc |
| 961 | tdcvdsytct cpagfsgihc enntpdctes scfnggtcvd ginsftclcp pgftgsycqh |
| 1021 | dvnecdsqpc lhggtcqdgc gsyrctcpqg ytgpncqnlv hwcdsspckn ggkcwqthtq |
| 1081 | yrcecpsgwt glycdvpsvs cevaaqrqgv dvarlcqhgg 1cvdagnthh crcqagytgs |
| 1141 | ycedlvdecs pspcqngatc tdylggysck cvagyhgvnc seeideclsh pcqnggtcld |
| 1201 | lpntykcscp rgtqgvhcei nvddenppvd pvsrspkcfn ngtcvdqvgg ysctcppgfv |
| 1261 | gercegdvne clsnpcdarg tqncvqrvnd fhcecraght grrcesving ckgkpckngg |
| 1321 | tcavasntar gfickcpagf egatcendar tcgslrclng gtcisgprsp tclclgpftg |
| 1381 | pecqfpassp clggnpcynq gtceptsesp fyrclcpakf ngllchildy sfgggagrdi |
| 1441 | ppplieeace lpecqedagn kvcslqcnnh acgwdggdcs Infndpwknc tqslqcwkyf |
| 1501 | sdghcdsqen sagclfdgfd cqraegqcnp lydqyckdhf sdghcdqgen saecewdgld |
| 1561 | caehvperla agtlvvvvlm ppeqlrnssf hflrelsrvl htnvvfkrda hgqqmifpyy |
| 1621 | greeelrkhp ikraaegwaa pdallgqvka sllpggsegg rrrreldpmd vrgsivylei |
| 1681 | 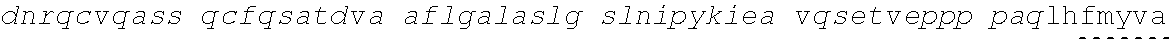 |
| 1741 | 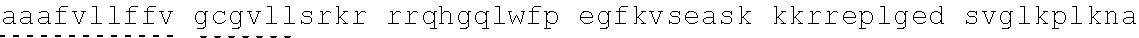 |
| 1801 | sdgalmddnq newgdedlet kkfrfeepvv lpdlddqtdh rqwtqqhlda adlrmsamap |
| 1861 | tppqgevdad cmdvnvrgpd gftplmiasc sgggletgns eeeedapavi sdfiyqgasl |
| 1921 | hnqtdrtget alhlaarysr sdaakrllea sadaniqdnm grtplhaavs adaqgvfqil |
| 1891 | irnratdlda rmhdgttpli laarlavegm ledlinshad vnavddlgks alhwaaavnn |
| 2041 | vdaavvllkn gankdmqnnr eetplflaar egsyetakvl ldhfanrdit dhmdrlprdi |
| 2101 | agermhhdiv rlldeynlvr spqlhgaplg gtptlspplc spngylgslk pgvqgkkvrk |
| 2161 | psskglacgs keakdlkarr kksqdgkgcl ldssgmlspv dslesphgyl sdvasppllp |
| 2221 | spfqqspsvp Inhlpgmpdt hlgighlnva akpemaalgg ggrlafetgp prlshlpvas |
| 2281 | gtstvlgsss ggalnftvgg stslngqcew lsrlqsgmvp nqynplrgsv apgplstqap |
| 2341 | slqhgmvgpl hsslaasals qmmsyqglps trlatqphlv qtqqvqpqnl qmqqqnlqpa |
| 2401 | niqqqqslqp pppppqphlg vssaasghlg rsflsgepsq advqplgpss lavhtilpqe |
| 2461 | spalptslps slvppvtaaq fltppsqhsy sspvdntpsh qlqvpehpfl tpspespdqw |
| 2521 | ssssphsnvs dwsegvsspp tsmqsqiari peafk |

Boldface = LNR segment;
underline = HD-N segment,
italics = HD-C segment,
dash underline = TM segment

In some embodiments, the Notch receptor polypeptide present in a P-selectin-specific chimeric receptor polypeptide of the present disclosure has a length of from 50 amino acids (aa) to 1000 aa, e.g., from 50 aa to 75 aa, from 75 aa to 100 aa, from 100 aa to 150 aa, from 150 aa to 200 aa, from 200 aa to 250 aa, from 250 a to 300 aa, from 300 aa to 350 aa, from 350 aa to 400 aa, from 400 aa to 450 aa, from 450 aa to 500 aa, from 500 aa to 550 aa, from 550 aa to 600 aa, from 600 aa to 650 aa, from 650 aa to 700 aa, from 700 aa to 750 aa, from 750 aa to 800 aa, from 800 aa to 850 aa, from 850 aa to 900 aa, from 900 aa to 950 aa, or from 950 aa to 1000 aa. In some embodiments, the Notch receptor polypeptide present in a P-selectin-specific chimeric receptor polypeptide of the present disclosure has a length of from 300 aa to 400 aa. In some embodiments, the Notch receptor polypeptide present in a P-selectin-specific chimeric receptor polypeptide of the present disclosure has a length of from 300 aa to 350 aa. In some embodiments, the Notch receptor polypeptide present in a P-selectin-specific chimeric receptor polypeptide of the present disclosure has a length of from 300 aa to 325 aa. In some embodiments, the Notch receptor polypeptide present in a P-selectin-specific chimeric receptor polypeptide of the present disclosure has a length of from 350 aa to 400 aa. In some embodiments, the Notch receptor polypeptide present in a P-selectin-specific chimeric receptor polypeptide of the present disclosure has a length of from 750 aa to 850 aa. In some embodiments, the Notch receptor polypeptide present in a P-selectin-specific chimeric receptor polypeptide of the present disclosure has a length of from 50 aa to 75 aa. In some embodiments, the Notch receptor polypeptide present in a P-selectin-specific chimeric receptor polypeptide of the present disclosure has a length of from 310 aa to 330 aa, e.g., 310 aa, 311 aa, 312 aa, 313 aa, 314 aa, 315 aa, 316 aa, 317 aa, 318 aa, 319 aa, 320 aa, 321 aa, 322 aa, 323 aa, 324 aa, 325 aa, 326 aa, 327 aa, 328 aa, 329 aa, or 330 aa. In some embodiments, the Notch receptor polypeptide present in a P-selectin-specific chimeric receptor polypeptide of the present disclosure has a length of 326 aa.

In some embodiments, the Notch receptor polypeptide present in a P-selectin-specific chimeric receptor polypeptide of the present disclosure comprises an amino acid sequence having at least 75%, at least 80%, at least 85%, at least 90%, at least 95%, at least 98%, at least 99%, or 100%, amino acid sequence identity to the following amino acid sequence:

(SEQ ID NO: 38)
IPYKIEAVKSEPVEPPLPSQLHLMYVAAAAFVLLFFVGCGVLLSRKRRR;

where the TM domain is underlined; where the Notch receptor polypeptide comprises an S2 proteolytic cleavage site and an S3 proteolytic cleavage site; where the Notch receptor polypeptide has a length of from 49 amino acids (aa) to 65 aa, e.g., 49, 50, 51, 52, 53, 54, 55, 56, 57, 58, 59, 60, 61, 62, 63, 64, or 65 aa.

In some embodiments, the Notch receptor polypeptide present in a P-selectin-specific chimeric receptor polypeptide of the present disclosure comprises, in order from N-terminus to C-terminus: i) a LNR segment; ii) an HD-N segment, iii) an HD-C segment; and iv) a TM domain.

An LNR segment can comprise an amino acid sequence having at least 75%, at least 80%, at least 85%, at least 90%, at least 95%, at least 98%, at least 99%, or 1000%, amino acid sequence identity to amino acids 1442-1562 of the amino acid sequence of SEQ ID NO: 37; and can have a length of from 90 amino acids to 150 amino acids, e.g., from 90 amino acids (aa) to 100 aa, from 100 aa to 110 aa, from 110 aa to 120 aa, from 120 aa to 130 aa, from 130 aa to 140 aa, or from 140 aa to 150 aa. In some embodiments, an LNR segment comprises an amino acid sequence having at least 75%, at least 80%, at least 85%, at least 90%, at least 95%, at least 98%, at least 99%, or 100%, amino acid sequence identity to amino acids 1442-1562 of the amino acid sequence of SEQ ID NO: 37; and has a length of from 115 aa to 125 aa, e.g., 115, 116, 117, 118, 119, 120, 121, 122, 123, 124, or 125 aa.

An LNR segment can comprise an amino acid sequence having at least 75%, at least 80%, at least 85%, at least 90% c, at least 95%, at least 98%, at least 99%, or 100%, amino acid sequence identity to the following amino acid sequence: PPQIEEACELPECQVDAGNKVCNLQCNNHACGWDGDCSLNFNDPWKNCTQSLQ CWKYFSDGHCDSQCNSAGCLFDGFDCQLTEGQCNPLYDQYCKDHFSDGHCDQGC NSAECEWDGLDC (SEQ ID NO: 39); and can have a length of from 118 to 122 amino acids (e.g., 118, 119, 120, 121, or 122 amino acids).

An HD-N segment can comprise an amino acid sequence having at least 75%, at least 80%, at least 85%, at least 90%, at least 95%, at least 98%, at least 99%, or 100%, amino acid sequence identity to amino acids 1563-1664 of the amino acid sequence of SEQ ID NO: 37, and can have a length of from 90 amino acids (aa) to 110 aa, e.g., 90 aa to 95 aa, 95 aa to 100 aa, 100 aa to 105 aa, or 105 aa to 110 aa. In some embodiments, an HD-N segment comprises an amino acid sequence having at least 75%, at least 80%, at least 85%, at least 90%, at least 95%, at least 98%, at least 99%, or 100%, amino acid sequence identity to amino acids 1563-1664 of the amino acid sequence of SEQ ID NO: 37; and has a length of from 95 aa to 105 aa, e.g., 95, 96, 98, 98, 99, 100, 101, 102, 103, 104, or 105 aa.

An HD-C segment can comprise an amino acid sequence having at least 75%, at least 80%, at least 85%, at least 90%, at least 95%, at least 98%, at least 99%, or 1000%, amino acid sequence identity to amino acids 1665-1733 of the amino acid sequence of SEQ ID NO: 37; and can have a length of from 60 amino acids (aa) to 80 aa, e.g., from 60 aa to 65 aa, from 65 aa to 70 aa, from 70 aa to 75 aa, or from 75 aa to 80 aa in some embodiments, an HD-C segment comprises an amino acid sequence having at least 75%, at least 80%, at least 85%, at least 90%, at least 95%, at least 98%, at least 99%, or 100%, amino acid sequence identity to amino acids 1665-1733 of the amino acid sequence of SEQ ID NO: 37; and has a length of from 65 amino acids to 75 amino acids, e.g., 65, 66, 67, 68, 69, 70, 71, 72, 73, 74, or 75 amino acids.

An HD segment (HD-N plus HD-C) can comprise an amino acid sequence having at least 75%, at least 80%, at least 85%, at least 90%, at least 95%, at least 98%, at least 99%, or 100%, amino acid sequence identity to the following amino acid sequence: AAGTLVLVVLLPPDQLRNNSFHFLRELSHVLHTNVVFKRDAQGQQMIFPYYGHEEE LRKHPIKRSTVGWATSSLLPGTSGGRQRRELDPMDIRGSIVYLEDNRQCVQSSSQC FQSATDVAAFLGALASLGSLNIPYKIEAVKSEPVEPPLP (SEQ ID NO: 40); and can have a length of 150, 151, 152, 153, or 154 amino acids.

A transmembrane segment can comprise an amino acid sequence having at least 75%, at least 80%, at least 85%, at least 90%, at least 95%, at least 98%, at least 99%, or 100%, amino acid sequence identity to amino acids 1736 to 1756 of the amino acid sequence of SEQ ID NO: 37; and can have a length of from 15 amino acids (aa) to 25 amino acids, e.g., 15, 16, 17, 18, 29, 20, 21, 22, 23, 24, or 25 amino acids.

A transmembrane segment can comprise an amino acid sequence having at least 75%, at least 80%, at least 85%, at least 90%, at least 95%, at least 98%, at least 99%, or 100%, amino acid sequence identity to the following amino acid sequence: HLMYVAAAAFVLLFFVGCGVLLS (SEQ ID NO: 41); and can have a length of 21, 22, 23, 24, or 25 amino acids.

In some embodiments, a Notch receptor polypeptide has a length of from about 310 amino acids (aa) to about 330 aa (e.g., 310 aa, 311 aa, 312 aa, 313 aa, 314 aa, 315 aa, 316 aa, 317 aa, 318 aa, 319 aa, 320 aa, 321 aa, 322 aa, 323 aa, 324 aa, 325 aa, 326 aa, 327 aa, 328 aa), and comprises an amino acid sequence having at least 75%, at least 80%, at least 85%, at least 90%, at least 95%, at least 98%, at least 99%, or 100%, amino acid sequence identity to amino acids 1442-1756 of the amino acid sequence of SEQ ID NO: 37.

In some embodiments, a Notch receptor polypeptide comprises an amino acid sequence having at least 75%, at least 80%, at least 85%, at least 90%, at least 95%, at least 98%, at least 99%, or 100%, amino acid sequence identity to the following amino acid sequence: PPQIEEACELPECQVDAGNKVCNLQCNNHACGWDGCDCSLNFNDPWKNCTQSLQ CWKYFSDGHCDSQCNSAGCLFDGFDCQLTEGQCNPLYDQYCKDHFSDGHCDQGC NSAECEWDGLDCAEHVPERLAAGTLVLVVLLPPDQLRNNSFHFLRELSHVLHTNV VFKRDAQGQQMIFPYYGHEEELRKHPIKRSTVGWATSSLLPGTSGGRQRRELDPMD IRGSIVYLEIDNRQCVQSSSQCFQSATDVAAFLGALASLGSLNIPYKIEAVKSEPVEPP LPSQLHLMYVAAAAFVLLFFVGCGVLLS (SEQ ID NO: 42); and has a length of from 300 amino acids to 310 amino acids (e.g., 300, 301, 302, 303, 304, 305, 306, 307, 308, 309, or 310 amino acids).

In some embodiments, a Notch receptor polypeptide comprises an amino acid sequence having at least 75%, at least 80%, at least 85%, at least 90%, at least 95%, at least 98%, at least 99%, or 100%, amino acid sequence identity to the following amino acid sequence:

(SEQ ID NO: 43)
ILDYSFTGGAGRDIPPPQIEEACELPECQVDAGNKVCNLQCNNHA
CGWDGGDCSLNFNDPWKNCTQSLQCWKYFSDGHCDSQCNSAGCLF
DGFDCQLTEGQCNPLYDQYCKDHFSDGHCDQGCNSAECEWDGLDC
AEHVPERLAAGTLVLVVLLPPDQLRNNSFHFLRELSHVLHTNVV
FKRDAQGQQMIFPYYGHEEELRKHPIKRSTVGWATSSLLPGTSGG
RQRRELDPMDIRGSIVYLEIDNRQCVQSSSQCFQSATDVAAFLGA
LASLGSLNIPYKIEAVKSEPVEPPLPSQLHLMYVAAAAFVLLFFV
GCGVLLS;

and has a length of from 350 amino acids to 370 amino acids (e.g., 350 351, 352, 353, 354, 355, 356, 357, 358, 359, 360, 361, 362, 363, 364, 365, 366, 367, 368, 369, or 370 amino acids).

In some embodiments, the Notch receptor polypeptide present in a P-selectin-specific chimeric receptor polypeptide of the present disclosure comprises, in order from N-terminus to C-terminus: i) a single EGF repeat; ii) an LNR segment; iii) an HD-N segment, iv) an HD-C segment; and v) a TM domain.

An EGF repeat can comprises an amino acid sequence having at least 75%, at least 80%, at least 85%, at least 90%, at least 95%, at least 98%, at least 99%, or 100%, amino acid sequence identity to amino acids 1390 to 1430 of the amino acid sequence of SEQ ID NO: 37; and can have a length of from 35 amino acids (aa) to 45 aa (e.g., 35, 36, 37, 38, 39, 40, 41, 42, 43, 44, or 45 aa).

An EGF repeat can comprise an amino acid sequence having at least 75%, at least 80%, at least 85%, at least 90%, at least 95%, at least 98%, at least 99%, or 100%, amino acid sequence identity to the following sequence PCVGSNPCYNQGTCEPTSENPFYRCLCPAKFNGLLCH (SEQ ID NO: 44); and can have a length of 35 amino acids to 40 amino acids (e.g., 35, 36, 37, 38, 39, or 40 amino acids.

An LNR segment can comprise an amino acid sequence having at least 75%, at least 80%, at least 85%, at least 90%, at least 95%, at least 98%, at least 99%, or 1(00, amino acid sequence identity to amino acids 1442-1562 of the amino acid sequence of SEQ ID NO: 37, and can have a length of from 90 amino acids to 150 amino acids, e.g., from 90 amino acids (aa) to 100 aa, from 100 aa to 110 aa, from 110 aa to 120 aa, from 120 aa to 130 aa, from 130 aa to 140 aa, or from 140 aa to 150 aa.

In some embodiments, an LNR segment comprises an amino acid sequence having at least 75%, at least 80%, at least 85%, at least 90%, at least 95%, at least 98%, at least 99%, or 100%, amino acid sequence identity to amino acids 1442-1562 of the amino acid sequence of SEQ ID NO: 37; and has a length of from 115 aa to 125 aa, e.g., 115, 116, 117, 118, 119, 120, 121, 122, 123, 124, or 125 aa.

An LNR segment can comprise an amino acid sequence having at least 75%, at least 80%, at least 85%, at least 90%, at least 95%, at least 98%, at least 990, or 100%, amino acid sequence identity to the following amino acid sequence: PPQIEEACELPECQVDAGNKVCNLQCNNHACGWDGGDCSLNFNDPWKNCTQSLQ CWKYFSDGHCDSQCNSAGCLFDGFDCQLTEGQCNPLYDQYCKDHFSDGHCDQGC NSAECEWDGLDC (SEQ 11) NO: 39); and can have a length of from 118 to 122 amino acids (e.g., 118, 119, 120, 121, or 122 amino acids).

An HD-N segment can comprise an amino acid sequence having at least 75%, at least 80%, at least 85%, at least 90%, at least 95%, at least 98%, at least 99%, or 1001%, amino acid sequence identity to amino acids 1563-1664 of the amino acid sequence of SEQ ID NO: 37, and can have a length of from 90 amino acids (aa) to 110 aa, e.g., 90 aa to 95 aa, 95 aa to 100 aa, 100 aa to 105 aa, or 105 aa to 110 aa. In some embodiments, an HD-N segment comprises an amino acid sequence having at least 75%, at least 80%, at least 85%, at least 90%, at least 95%, at least 98%, at least 99%, or 1000%, amino acid sequence identity to amino acids 1563-1664 of the amino acid sequence of SEQ ID NO: 37, and has a length of from 95 aa to 105 aa, e.g., 95, 96, 98, 98, 99, 100, 101, 102, 103, 104, or 105 aa.

An HD-C segment can comprise an amino acid sequence having at least 75%, at least 80%, at least 85%, at least 90%, at least 95%, at least 98%, at least 99%, or 100%, amino acid sequence identity to amino acids 1665-1733 of the amino acid sequence of SEQ ID NO: 37, and can have a length of from 60 amino acids (aa) to 80 aa, e.g., from 60 aa to 65 aa, from 65 aa to 70 aa, from 70 aa to 75 aa, or from 75 aa to 80 aa. In some embodiments, an HD-C segment comprises an amino acid sequence having at least 75%, at least 80%, at least 85%, at least 90%, at least 95%, at least 98%, at least 99%, or 100%, amino acid sequence identity to amino acids 1665-1733 of the amino acid sequence of SEQ ID NO: 37, and has a length of from 65 amino acids to 75 amino acids, e.g., 65, 66, 67, 68, 69, 70, 71, 72, 73, 74, or 75 amino acids.

An HD segment can comprise an amino acid sequence having at least 75%, at least 80%, at least 85%, at least 90%, at least 95%, at least 98%, at least 99%, or 100%, amino acid sequence identity to the following amino acid sequence: AAGTLVLVVLLPPDQLRNNSFHFLRELSHVLHTNVVFKRDAQGQQMIFPYYGHEEE LRKHPLKRSTVGWATSSLLPGTSGGRQRRELDPMDIRGSIVYLEIDNRQCVQSSSQC FQSATDVAAFLGALASLGSLNTPYKIEAVKSEPVEPPLP (SEQ ID NO: 40), and can have a length of 150, 151, 152, 153, or 154 amino acids.

A transmembrane segment can comprise an amino acid sequence having at least 75%, at least 80%, at least 85%, at least 90%, at least 95%, at least 98%, at least 99%, or 100%, amino acid sequence identity to amino acids 1736 to 1756 of the amino acid sequence of SEQ ID NO: 37, and can have a length of from 15 amino acids (aa) to 25 amino acids, e.g., 15, 16, 17, 18, 29, 20, 21, 22, 23, 24, or 25 amino acids.

A transmembrane segment can comprise an amino acid sequence having at least 75%, at least 80%, at least 85%, at least 90%, at least 95%, at least 98%, at least 99%, or 100%, amino acid sequence identity to the following amino acid sequence: HLMYVAAAAFVLLFFVGCGVLLS (SEQ ID NO: 41), and can have a length of 21, 22, 23, 24, or 25 amino acids.

In some embodiments, a Notch receptor polypeptide has a length of from about 360 amino acids (aa) to about 375 aa (e.g., 360 aa, 361 aa, 362 aa, 363 aa, 364 aa, 365 aa, 366 aa, 367 aa, 368 aa, 369 aa, 370 aa, 371 aa, 372 aa, 373 aa, 374 aa, or 375 aa), and comprises an amino acid sequence having at least 75%, at least 80%, at least 85%, at least 90%, at least 95%, at least 98%, at least 99%, or 100%, amino acid sequence identity to amino acids 1390-1756 of the amino acid sequence of SEQ ID NO: 37.

In some embodiments, a Notch receptor polypeptide comprises a synthetic linker. For example, In some embodiments, a Notch receptor polypeptide comprises, in order from N-terminus to C-terminus: i) a synthetic linker, ii) an EGF repeat; iii) an LNR segment; iv) an HD-N segment, v) an HD-C segment; and vi) a TM domain.

A synthetic linker can have a length of from about 10 amino acids (aa) to about 200 aa, e.g., from 10 aa to 25 aa, from 25 aa to 50 aa, from 50 aa to 75 aa, from 75 aa to 100 aa, from 100 aa to 125 aa, from 125 aa to 150 aa, from 150 aa to 175 aa, or from 175 aa to 200 aa. A synthetic linker can have a length of from 10 aa to 30 aa, e.g., 10, 11, 12, 13, 14, 15, 16, 17, 18, 19, 20, 21, 22, 23, 24, 25, 26, 27, 28, 29, or 30 aa. A synthetic linker can have a length of from 30 aa to 50 aa, e.g., from 30 aa to 35 aa, from 35 an to 40 aa, from 40 aa to 45 aa, or from 45 aa to 50 aa.

In some embodiments, the Notch receptor polypeptide present in a P-selectin-specific chimeric receptor polypeptide of the present disclosure comprises, in order from N-terminus to C-terminus: i) from two to eleven EGF repeats; ii) an LNR segment; iii) an HD-N segment, iv) an HD-C segment; and v) a TM domain.

In some embodiments, the Notch receptor polypeptide present in a P-selectin-specific chimeric receptor polypeptide of the present disclosure comprises, in order from N-terminus to C-terminus: i) two EGF repeats; ii) an LNR segment; iii) an HD-N segment, iv) an HD-C segment; and v) a TM domain. In some embodiments, the Notch receptor polypeptide present in a P-selectin-specific chimeric receptor polypeptide of the present disclosure comprises, in order from N-terminus to C-terminus: i) three EGF repeats; ii) an LNR segment; iii) an HD-N segment, iv) an HD-C segment; and v) a TM domain. In some embodiments, the Notch receptor polypeptide present in a P-selectin-specific chimeric receptor polypeptide of the present disclosure comprises, in order from N-terminus to C-terminus: i) four EGF repeats; ii) an LNR segment; iii) an HD-N segment, iv) an I-D-C segment; and v) a TM domain. In some embodiments, the Notch receptor polypeptide present in a P-selectin-specific chimeric receptor polypeptide of the present disclosure comprises, in order from N-terminus to C-terminus: i) five EGF repeats; ii) an LNR segment, iii) an HD-N segment, iv) an HD-C segment; and v) a TM domain. In some embodiments, the Notch receptor polypeptide present in a P-selectin-specific chimeric receptor polypeptide of the present disclosure comprises, in order from N-terminus to C-terminus: i) six EGF repeats; ii) an LNR segment, iii) an HD-N segment, iv) an HD-C segment; and v) a TM domain. In some embodiments, the Notch receptor polypeptide present in a P-selectin-specific chimeric receptor polypeptide of the present disclosure comprises, in order from N-terminus to C-terminus: i) seven EGF repeats; ii) an LNR segment; iii) an HD-N segment, iv) an HD-C segment; and v) a TM domain in some embodiments, the Notch receptor polypeptide present in a P-selectin-specific chimeric receptor polypeptide of the present disclosure comprises, in order from N-terminus to C-terminus: i) eight EGF repeats; ii) an LNR segment; iii) an HD-N segment, iv) an HD-C segment; and v) a TM domain. In some embodiments, the Notch receptor polypeptide present in a P-selectin-specific chimeric receptor polypeptide of the present disclosure comprises, in order from N-terminus to C-terminus: i) nine EGF repeats; ii) an LNR segment, iii) an HD-N segment, iv) an HD-C segment; and v) a TM domain. In some embodiments, the Notch receptor polypeptide present in a P-selectin-specific chimeric receptor polypeptide of the present disclosure comprises, in order from N-terminus to C-terminus: i) ten EGF repeats; ii) an LNR segment; iii) an HD-N segment, iv) an HD-C segment, and v) a TM domain. In some embodiments, the Notch receptor polypeptide present in a P-selectin-specific chimeric receptor polypeptide of the present disclosure comprises, in order from N-terminus to C-terminus: i) eleven EGF repeats; ii) an LNR segment; iii) an HD-N segment, iv) an HD-C segment; and v) a TM domain.

An EGF repeat can comprise an amino acid sequence having at least 75%, at least 80%, at least 85%, at least 90%, at least 95%, at least 98%, at least 99%, or 100%/0, amino acid sequence identity to amino acids 1390 to 1430 of the amino acid sequence of SEQ ID NO: 37; and can have a length of from 35 amino acids (aa) to 45 aa (e.g., 35, 36, 37, 38, 39, 40, 41, 42, 43, 44, or 45 aa).

An EGF repeat can comprise an amino acid sequence having at least 75%, at least 80%, at least 85%, at least 90%, at least 95%, at least 98%, at least 99%, or 1000%, amino acid sequence identity to amino acids 869-905 (DINECVLSPCRHGASCQNTHGGYRCHCQAGYSGRNCE; SEQ ID NO: 45) of the amino acid sequence of SEQ ID NO: 37; and can have a length of from 35 amino acids to about 40 amino acids (aa) (e.g., 35, 36, 37, 38, 39, or 40 aa).

An EGF repeat can comprise an amino acid sequence having at least 75%, at least 80%, at least 85%, at least 90% c, at least 95%, at least 98%, at least 99%, or 100%, amino acid sequence identity to amino acids 907-943 (DIDDCRPNPCHNGGSCTDGINTAFCDCLPGFRGTFCE; SEQ ID NO: 46) of the amino acid sequence of SEQ ID NO: 37; and can have a length of from 35 amino acids to about 40 amino acids (aa) (e.g., 35, 36, 37, 38, 39, or 40 aa).

An EGF repeat can comprise an amino acid sequence having at least 75%, at least 80%, at least 85%, at least 90%, at least 95%, at least 98%, at least 99%, or 100%, amino acid sequence identity to amino acids 945-981 (DINECASDPCRNGANCTDCVDSYTCTCPAGFSGIHCE; (SEQ ID NO: 47) of the amino acid sequence of SEQ ID NO: 37; and can have a length of from 35 amino acids to about 40 amino acids (aa) (e.g., 35, 36, 37, 38, 39, or 40 aa).

An EGF repeat can comprise an amino acid sequence having at least 75%, at least 80%, at least 85%, at least 90%, at least 95%, at least 98%, at least 99%, or 100/0, amino acid sequence identity to amino acids 988-1019 (TESSCFNGGTCVDGINSFTCLCPPGFTGSYCQ, SEQ ID NO: 48) of the amino acid sequence of SEQ ID NO: 37; and can have a length of from 30 amino acids (aa) to 35 aa (e.g., 30, 31, 32, 33, 34, or 35 aa).

An EGF repeat can comprise an amino acid sequence having at least 75%, at least 80%, at least 85%, at least 90%, at least 95%, at least 98%, at least 99%, or 100%, amino acid sequence identity to amino acids 1021-1057 (DVNECDSQPCLHGGTCQDGCGSYRCTCPQGYTGPNCQ; SEQ ID NO: 49) of the amino acid sequence of SEQ ID NO: 37; and can have a length of from 35 amino acids to about 40 amino acids (aa) (e.g., 35, 36, 37, 38, 39, or 40 aa).

An EGF repeat can comprise an amino acid sequence having at least 75%, at least 80%, at least 85%, at least 90%, at least 95%, at least 98%, at least 99%, or 1000%, amino acid sequence identity to amino acids 1064-1090 (DSSPCKNGGKCWQTHTQYRCECPSGWT; SEQ ID NO: 50) of the amino acid sequence of SEQ ID NO: 37; and can have a length of from 25 amino acids (aa) to 30 aa, e.g., 25, 26, 27, 28, 29, or 30 aa.

An EGF repeat can comprise an amino acid sequence having at least 75%, at least 80%, at least 85%, at least 90%, at least 95%, at least 98%, at least 99%, or 100%, amino acid sequence identity to amino acids 1146-1180 (LVDECSPSPCQNGATCTDYLGGYSCKCVAGYFHGVNC, SEQ ID NO: 51) of the amino acid sequence of SEQ ID NO: 37; and can have a length of from 35 amino acids to about 40 amino acids (aa) (e.g., 35, 36, 37, 38, 39, or 40 aa).

An EGF repeat can comprise an amino acid sequence having at least 75%, at least 80%, at least 85%, at least 90%, at least 95%, at least 98%, at least 99%, or 1000%, amino acid sequence identity to amino acids 1184-1219 (IDECLSHPCQNGGTCLDLPNTYKCSCPRGTQGVHCE; SEQ ID NO: 52) of the amino acid sequence of SEQ ID NO: 37, and can have a length of from 35 amino acids to about 40 amino acids (aa) (e.g., 35, 36, 37, 38, 39, or 40 aa).

An EGF repeat can comprise an amino acid sequence having at least 75%, at least 80%, at least 85%, at least 90%, at least 95%, at least 98%, at least 99%, or 100%, amino acid sequence identity to amino acids 1238-1265 (CFNNGTCVDQVGGYSCTCPPGFVGERCE, SEQ ID NO: 53) of the amino acid sequence of SEQ ID NO: 37; and can have a length of from 25 amino acids (aa) to 30 aa, e.g., 25, 26, 27, 28, 29, or 30 aa.

An EGF repeat can comprise an amino acid sequence having at least 75%, at least 80%, at least 85%, at least 90, at least 95%, at least 98%, at least 99%, or 100%, amino acid sequence identity to amino acids 1267-1305 (DVNECLSNPCDARGTQNCVQRVNDFHCECRAGHTGRRCE; (SEQ ID NO: 54) of the amino acid sequence of SEQ ID NO: 37; and can have a length of from 35 amino acids to about 40 amino acids (aa) (e.g., 35, 36, 37, 38, 39, or 40 aa).

An EGF repeat can comprise an amino acid sequence having at least 75%, at least 80%, at least 85%, at least 90%, at least 95%, at least 98%, at least 99%, or 100/0, amino acid sequence identity to the following sequence PCVGSNPCYNQGTCEPTSENPFYRCLCPAKFNGLLCH (SEQ ID NO: 44); and can have a length of 35 amino acids to 40 amino acids (e.g., 35, 36, 37, 38, 39, or 40 amino acids.

An LNR segment can comprise an amino acid sequence having at least 75%, at least 80%, at least 85%, at least 90%, at least 95%, at least 98%, at least 99%, or 100%, amino acid sequence identity to amino acids 1442-1562 of the amino acid sequence of SEQ ID NO: 37; and can have a length of from 90 amino acids to 150 amino acids, e.g., from 90 amino acids (aa) to 100 aa, from 100 aa to 110 aa, from 110 aa to 120 aa, from 120 aa to 130 aa, from 130 aa to 140 aa, or from 140 aa to 150 aa. In some embodiments, an LNR segment comprises an amino acid sequence having at least 75%, at least 80%, at least 85%, at least 90%, at least 95%, at least 98%, at least 99%, or 100%, amino acid sequence identity to amino acids 1442-1562 of the amino acid sequence of SEQ ID NO: 37; and has a length of from 115 aa to 125 aa, e.g., 115, 116, 117, 118, 119, 120, 121, 122, 123, 124, or 125 aa.

An LNR segment can comprise an amino acid sequence having at least 75%, at least 80%, at least 85%, at least 90%, at least 95%, at least 98%, at least 99%, or 100%, amino acid sequence identity to the following amino acid sequence: PPQIEEACELPECQVDAGNKVCNLQCNNHACGWDGGDCSLNFNDPWKNCTQSLQ CWKYFSDGHCDSQCNSAGCLFDGFDCQLTEGQCNPLYDQYCKDHFSDGHCDQGC NSAECEWDGLDC (SEQ ID NO: 39); and can have a length of from 118 to 122 amino acids (e.g., 118, 119, 120, 121, or 122 amino acids).

An HD-N segment can comprise an amino acid sequence having at least 75%, at least 80%, at least 85%, at least 90%, at least 95%, at least 98%, at least 99%, or 100%, amino acid sequence identity to amino acids 1563-1664 of the amino acid sequence of SEQ ID NO: 37; and can have a length of from 90 amino acids (aa) to 110 aa, e.g., 90 aa to 95 aa, 95 aa to 100 aa, 100 aa to 105 aa, or 105 aa to 110 aa. In some embodiments, an HD-N segment comprises an amino acid sequence having at least 75%, at least 80%, at least 85%, at least 90%, at least 95%, at least 98%, at least 99%, or 100%, amino acid sequence identity to amino acids 1563-1664 of the amino acid sequence of SEQ ID NO: 37; and has a length of from 95 aa to 105 aa, e.g., 95, 96, 98, 98, 99, 100, 101, 102, 103, 104, or 105 aa.

An HD-C segment can comprise an amino acid sequence having at least 75%, at least 80%, at least 85%, at least 90%, at least 95%, at least 98%, at least 99%, or 1000%, amino acid sequence identity to amino acids 1665-1733 of the amino acid sequence of SEQ ID NO: 37; and can have a length of from 60 amino acids (aa) to 80 aa, e.g., from 60 aa to 65 aa, from 65 aa to 70 aa, from 70 aa to 75 aa, or from 75 aa to 80 aa. In some embodiments, an HD-C segment comprises an amino acid sequence having at least 75%, at least 80%, at least 85%, at least 90%, at least 95%, at least 98%, at least 99%, or 1001%, amino acid sequence identity to amino acids 1665-1733 of the amino acid sequence of SEQ ID NO: 37; and has a length of from 65 amino acids to 75 amino acids, e.g., 65, 66, 67, 68, 69, 70, 71, 72, 73, 74, or 75 amino acids.

An HD segment (HD-N plus HD-C) can comprise an amino acid sequence having at least 75%, at least 80%, at least 85%, at least 90%, at least 95%, at least 98%, at least 99%, or 100%, amino acid sequence identity to the following amino acid sequence: AAGTLVLVVLLPPDQLRNNSFHFLRELSHVLHTNVVFKRDAQGQQMIFPYYGHEEE LRKHPIKRSTVGWATSSLLPGTSGGRQRRELDPMDIRGSIVYLEIDNRQCVQSSSQC FQSATDVAAFLGALASLGSLNIPYKIEAVKSEPVEPPLP (SEQ ID NO: 40); and can have a length of 150, 151, 152, 153, or 154 amino acids.

A transmembrane segment can comprise an amino acid sequence having at least 75%, at least 80%, at least 85%, at least 90%, at least 95%, at least 98%, at least 99%, or 100%, amino acid sequence identity to amino acids 1736 to 1756 of the amino acid sequence of SEQ ID NO: 37; and can have a length of from 15 amino acids (aa) to 25 amino acids, e.g., 15, 16, 17, 18, 29, 20, 21, 22, 23, 24, or 25 amino acids.

A transmembrane segment can comprise an amino acid sequence having at least 75%, at least 80%, at least 85%, at least 90%, at least 95%, at least 98%, at least 99%, or 100%, amino acid sequence identity to the following amino acid sequence: HLMYVAAAAFVLLFFVGCGVLLS (SEQ ID NO: 41); and can have a length of 21, 22, 23, 24, or 25 amino acids.

In some embodiments, a Notch receptor polypeptide has a length of from about 490 amino acids (aa) to about 900 aa, and comprises an amino acid sequence having at least 75%, at least 80%, at least 85%, at least 90%, at least 95%, at least 98%, at least 99%, or 100%, amino acid sequence identity to: i) amino acids 1267-1756; ii) 1238-1756; iii) 1184-1756; iv) 1146-1756; v) 1064-1756; vi) 1021-1756; vii) 988-1756; viii) 945-1756; ix) 907-1756; or x) 869-1756, of the amino acid sequence of SEQ ID NO: 37.

In some embodiments, a Notch receptor polypeptide comprises a synthetic linker. For example, In some embodiments, a Notch receptor polypeptide comprises, in order from N-terminus to C-terminus: i) two to eleven EGF repeats; ii) a synthetic linker; iii) an LNR segment; iv) an HD-N segment, v) an HD-C segment; and vi) a TM domain.

A synthetic linker can have a length of from about 10 amino acids (aa) to about 200 aa, e.g., from 10 as to 25 aa, from 25 aa to 50 aa, from 50 aa to 75 aa, from 75 aa to 100 aa, from 100 aa to 125 aa, from 125 aa to 150 aa, from 150 aa to 175 aa, or from 175 aa to 200 aa. A synthetic linker can have a length of from 10 aa to 30 aa, e.g., 10, 11, 12, 13, 14, 15, 16, 17, 18, 19, 20, 21, 22, 23, 24, 25, 26, 27, 28, 29, or 30 aa. A synthetic linker can have a length of from 30 aa to 50 aa, e.g., from 30 aa to 35 aa, from 35 aa to 40 aa, from 40 aa to 45 aa, or from 45 aa to 50 aa.

In some embodiments, a Notch receptor polypeptide comprises, in order from N-terminus to C-terminus: i) an HD-C segment; and ii) a TM domain, where the Notch receptor polypeptide does not include an LNR segment. In some embodiments, the LNR segment is replaced with a heterologous polypeptide.

An HD-C segment can comprise an amino acid sequence having at least 75%, at least 80%, at least 85%, at least 90%, at least 95%, at least 98%, at least 99%, or 1(00, amino acid sequence identity to amino acids 1665-1733 of the amino acid sequence of SEQ ID NO: 37, and can have a length of from 60 amino acids (aa) to 80 aa, e.g., from 60 aa to 65 aa, from 65 aa to 70 aa, from 70 aa to 75 aa, or from 75 aa to 80 aa. In some embodiments, an HD-C segment comprises an amino acid sequence having at least 75%, at least 80%, at least 85%, at least 90%, at least 95%, at least 98%, at least 99%, or 100%, amino acid sequence identity to amino acids 1665-1733 of the amino acid sequence of SEQ ID NO: 37, and has a length of from 65 amino acids to 75 amino acids, e.g., 65, 66, 67, 68, 69, 70, 71, 72, 73, 74, or 75 amino acids.

A transmembrane segment can comprise an amino acid sequence having at least 75%, at least 80%, at least 85%, at least 90%, at least 95%, at least 98%, at least 99%, or 100%, amino acid sequence identity to amino acids 1736 to 1756 of the amino acid sequence of SEQ ID NO: 37; and can have a length of from 15 amino acids (aa) to 25 amino acids, e.g., 15, 16, 17, 18, 29, 20, 21, 22, 23, 24, or 25 amino acids.

A transmembrane segment can comprise an amino acid sequence having at least 75%, at least 80%, at least 85%, at least 90%, at least 95%, at least 98%, at least 99%, or 100%, amino acid sequence identity to the following amino acid sequence: HLMYVAAAAFVLLFFVGCGVLLS (SEQ ID NO: 41); and can have a length of 21, 22, 23, 24, or 25 amino acids.

In some embodiments, a Notch receptor polypeptide comprises an amino acid sequence having at least 75%, at least 80%, at least 85%, at least 90%, at least 95%, at least 98%, at least 99%, or 100%, amino acid sequence identity to amino acids 1665 to 1756 of the amino acid sequence of SEQ ID NO: 37; and has a length of from 85 amino acids (aa) to 95 aa (e.g., 85, 86, 87, 88, 89, 90, 91, 92, 93, 94, or 95 aa).

In some embodiments, a Notch receptor polypeptide comprises an amino acid sequence having at least 75%, at least 80%, at least 85%, at least 90%, at least 95%, at least 98%, at least 99%, or 100%, amino acid sequence identity to amino acids 1665 to 1756 of the amino acid sequence of SEQ ID NO: 37; and comprises a heterologous polypeptide fused in-frame at the N-terminus of the Notch receptor polypeptide.

Ligand-Inducible Proteolytic Cleavage Sites. As noted above, a P-selectin-specific chimeric receptor polypeptide of the present disclosure comprises a Notch receptor polypeptide having a length of from 50 amino acids to 1000 amino acids, and comprising one or more ligand-inducible proteolytic cleavage sites. As discussed above, a P-selectin-specific chimeric receptor polypeptide of the present disclosure comprises: a) an extracellular domain that specifically binds P-selectin; b) a Notch receptor polypeptide comprising one or more ligand-inducible proteolytic cleavage sites; and c) an intracellular domain comprising a transcriptional activator, wherein binding of the extracellular domain to P-selectin induces cleavage of the heterologous receptor polypeptide at the one or more ligand-inducible proteolytic cleavage sites to release the intracellular domain (e.g., a Notch receptor polypeptide).

In some embodiments, the Notch receptor polypeptide includes only one ligand-inducible proteolytic cleavage site. In some embodiments, the Notch receptor polypeptide includes two ligand-inducible proteolytic cleavage sites. In some embodiments, the Notch receptor polypeptide includes three ligand-inducible proteolytic cleavage sites. For simplicity, ligand-inducible cleavage sites will be referred to herein as “S1,” “S2,” and “S3” ligand-inducible proteolytic cleavage sites.

In some embodiments, the Notch receptor polypeptide includes an S1 ligand-inducible proteolytic cleavage site. An S1 ligand-inducible proteolytic cleavage site can be located between the HD-N segment and the HD-C segment. In some embodiments, the S1 ligand-inducible proteolytic cleavage site is a furin-like protease cleavage site. A furin-like protease cleavage site can have the canonical sequence Arg-X-(Arg/Lys)-Arg, where X is any amino acid; the protease cleaves immediately C-terminal to the canonical sequence. For example, in some embodiments, an amino acid sequence comprising an S1 ligand-inducible proteolytic cleavage site can have the amino acid sequence GRRRRELDPM (SEQ ID NO. 55), where cleavage occurs between the “RE” sequence. As another example, an amino acid sequence comprising an S1 ligand-inducible proteolytic cleavage site can have the amino acid sequence RQRRELDPM (SEQ ID NO: 56), where cleavage occurs between the “RE” sequence.

In some embodiments, the Notch receptor polypeptide includes an S2 ligand-inducible proteolytic cleavage site. An S2 ligand-inducible proteolytic cleavage site can be located within the HD-C segment. In some embodiments, the S2 ligand-inducible proteolytic cleavage site is an ADAM-17-type protease cleavage site. An ADAM-17-type protease cleavage site can comprise an Ala-Val dipeptide sequence, where the enzyme cleaves between the Ala and the Val. For example, In some embodiments, amino acid sequence comprising an S2 ligand-inducible proteolytic cleavage site can have the amino acid sequence KIEAVKSE (SEQ ID NO: 57), where cleavage occurs between the “AV” sequence. As another example, an amino acid sequence comprising an S2 ligand-inducible proteolytic cleavage site can have the amino acid sequence KIEAVQSE (SEQ ID NO: 58), where cleavage occurs between the “AV” sequence.

In some embodiments, the Notch receptor polypeptide includes an S3 ligand-inducible proteolytic cleavage site. An S3 ligand-inducible proteolytic cleavage site can be located within the TM domain. In some embodiments, the S3 ligand-inducible proteolytic cleavage site is a gamma-secretase (γ-secretase) cleavage site. A γ-secretase cleavage site can comprise a Gly-Val dipeptide sequence, where the enzyme cleaves between the Gly and the Val. For example, In some embodiments, an S3 ligand-inducible proteolytic cleavage site has the amino acid sequence VGCGVLLS (SEQ ID NO: 59), where cleavage occurs between the “GV” sequence. In some embodiments, an S3 ligand-inducible proteolytic cleavage site comprises the amino acid sequence GCGVLLS (SEQ ID NO: 60).

In some embodiments, the Notch receptor polypeptide lacks an S1 ligand-inducible proteolytic cleavage site. In some embodiments, the Notch receptor polypeptide lacks an S2 ligand-inducible proteolytic cleavage site. In some embodiments, the Notch receptor polypeptide lacks an S3 ligand-inducible proteolytic cleavage site. In some embodiments, the Notch receptor polypeptide lacks both an S1 ligand-inducible proteolytic cleavage site and an S2 ligand-inducible proteolytic cleavage site. In some embodiments, the Notch receptor polypeptide includes an S3 ligand-inducible proteolytic cleavage site; and lacks both an S1 ligand-inducible proteolytic cleavage site and an S2 ligand-inducible proteolytic cleavage site.

Intracellular Domain A P-selectin-specific chimeric receptor polypeptide of the present disclosure comprises an intracellular domain that is released following binding of the P-selectin-specific chimeric receptor polypeptide to P-selectin, where binding of the P-selectin-specific chimeric receptor polypeptide to the P-selectin induces cleavage of an above-mentioned proteolytic cleavage site.

The intracellular domain comprises an amino acid sequence that is heterologous to the Notch receptor polypeptide. Specifically, the intracellular domain comprises an amino acid sequence that is not naturally present in a Notch receptor polypeptide.

In some embodiments, the intracellular domain, when released from the P-selectin-specific chimeric receptor polypeptide, provides for a change in transcription of a target gene. In some embodiments, the intracellular domain, when released from the P-selectin-specific chimeric receptor polypeptide, provides for an increase in the transcription of a target gene. The intracellular domain can be any of a wide variety of polypeptides, where examples include, but are not limited to, transcriptional activators.

In some embodiments, the intracellular domain comprises an amino acid sequence having at least 75%, at least 80%, at least 85%, at least 90%, at least 95%, at least 98%, at least 99%, or 100%, amino acid sequence identity to the GAL4 DNA binding domain amino acid sequence of LEIRAAFLRQRNTALRTEVAELEQEVQRLENEVSQYETRYGPLGGGKGSGSGGSCGS MKLLSSIEQACDICRLKKLKCSKEKPKCAKCLKNNWECRYSPKTKRSPLTRAHL TE VESRLERLEQLFLLIFPREDLDMILKMDSLQDIKALLTGLFVQDNVNKDAVTDRLAS VETDMPLTLRQHRISATSSSEESSNKGQRQLTVSAA (SEQ ID NO: 61); and has a length of from 200 amino acids to 210 amino acids (e.g., 200, 201, 202, 203, 204, 205, 206, 207, 208, 209, or 210 amino acids).

In some embodiments, the intracellular domain is a transcriptional activator. In some embodiments, the transcriptional activator is GAL4-VP16. In some embodiments, the transcriptional activator is GAL4-VP64. In some embodiments, the transcriptional activator is Tbx21. In some embodiments the transcriptional activator is an engineered protein, such as a zinc finger or TALE based DNA binding domain fused to an effector domain such as VP64 (transcriptional activation) A variety of other transcriptional transactivators are known in the art is suitable for use.

In some embodiments, the intracellular domain comprises an amino acid sequence having at least 75%, at least 80%, at least 85%, at least 90%, at least 95%, at least 98%, at least 99%, or 100%, amino acid sequence identity to the following GAL4-VP64 sequence:

(SEQ ID NO: 62)
MKLLSSIEQACDICRLKKLKCSKEKPKCAKCLKNNWECRYSPKTK
RSPLTRAHLTEVESRLERLEQLFLLIFPREDLDMILKMDSLQDIK
ALLTGLFVQDNVNKDAVTDRLASVETDMPLTLRQHRISATSSSEE
SSNKGQRQLTVSAAAGGSGGSGGSDALDDFDLDMLGSDALDDFDL
DMLGSDALDDFDLDMLGSDALDDEDLDMLGS;

and has a length of from 208 to 214 amino acids (e.g., 208, 209, 210, 211, 212, 213, or 214 amino acids).

One exemplary P-selectin-specific chimeric receptor polypeptide of the present disclosure is provided below:

(SEQ ID NO: 93)

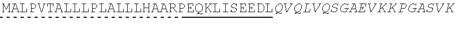

VSCKVSGYTFTSYDINWVRQAPGKGLEWMGWIYPGDGSIKYNEKFKGRVTMTVDKSTDT
AYMELSSLRSEDTAVYYCARRGEYGNYEGAMDYWGQGTLVTVSSGGGGSGGGGSGGG
GSDIQMTQSPSSLSASVGDRVTITCKASQSVDYDGHSYMNWYQQKPGKAPKLLIYAASNL
ESGVPSRFSSGSGSGTDFTLTISSSLQPEDFATYYCQQSDEENPLTFGGGTKVEIKRILDYSFT
GGAGRDIPPPQIEEACELPECQVDAGNKVCNLQCNNHACGWDGGDCSLNFND
PWKNCTQSLQCWKYFSDGHCDSQCNSAGCLFDGFDCQLTEGQCNPLYDQYC
KDHFSDGHCDQGCNSAECEWDGLDCAEHVPERLAAGTLVLVVLLPPDQLRNN
SFHFLRELSHVLHTNVVFKRDAQGQQMIFPYYGHEEELRKHPIKRSTVGWATS
SLLPGTSGGRQRRELDPMDIRGSIVYLEIDNRQCVQSSSQCFQSATDVAAFLGA
LASLGSLNIPYKIEAVKSEEPVEPPLPSQLHLMYVAAAAFVLLFFVGCGVLLSRK

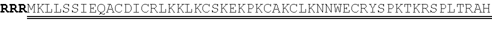

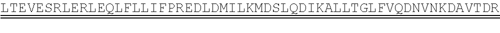

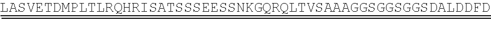

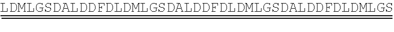

Dashed underline = Human IgH Signal Sequence; underlined =
myc tag; italics = VH and VL domains of P-selection
antigen binding fragement; Boldface = Notch;
double underline = Gal4

### Chimeric Antigen Receptors Induced by a Released Intracellular Domain of P-Selectin-Specific Chimeric Receptor Polypeptides of the Present Disclosure

In some embodiments, upon binding of the extracellular domain of the P-selectin-specific chimeric receptor polypeptide (e.g., a Notch receptor polypeptide) of the present disclosure to P-selectin, the intracellular domain is a polypeptide that is released and induces production, in a cell that expresses the P-selectin-specific chimeric receptor polypeptide (e.g., a Notch receptor polypeptide), of a gene product. For example, in some embodiments, the intracellular domain of a P-selectin-specific chimeric receptor polypeptide (e.g., a Notch receptor polypeptide) of the present disclosure, when released upon binding of the extracellular domain of the P-selectin-specific chimeric receptor polypeptide (e.g., a Notch receptor polypeptide) to P-selectin, induces production of a gene product (a polypeptide; a nucleic acid) in a cell expressing the P-selectin-specific chimeric receptor polypeptide (e.g., a Notch receptor polypeptide). In some embodiments, the gene product is a nucleic acid encoding a CAR or a CAR polypeptide. In some embodiments, a P-selectin-specific chimeric receptor (e.g., a Notch receptor) responsive to a P-selectin antigen induces the expression of a CAR responsive to a target antigen (i.e., antigen X) containing one or more intracellular components necessary for T cell activation exclusively in the tumor microenvironment.

In some embodiments, the intracellular domain of a P-selectin-specific chimeric receptor polypeptide (e.g., a Notch receptor polypeptide) of the present disclosure comprises a transcriptional activator (e.g., Gal4-VP). Accordingly, the transcriptional activator may be expressed within a cell from an expression cassette encoding a P-selectin-specific chimeric receptor polypeptide (e.g., a Notch receptor polypeptide) of the present disclosure. Additionally or alternatively, the cell expressing the P-selectin-specific chimeric receptor polypeptide (e.g., a Notch receptor polypeptide) further comprises a promoter that is responsive to the transcriptional activator (e.g., Gal4 promoter) and is operably linked to a nucleic acid encoding a CAR. In certain embodiments, the promoter that is responsive to the transcriptional activator (e.g., Gal4 promoter) may be present in an expression cassette that is distinct from the expression cassette encoding the P-selectin-specific chimeric receptor polypeptide (e.g., a Notch receptor polypeptide). In other embodiments, the promoter that is responsive to the transcriptional activator (e.g., Gal4 promoter) may be present in an expression cassette that is distinct from the expression cassette encoding the P-selectin-specific chimeric receptor polypeptide (e.g., a Notch receptor polypeptide).

An exemplary Gal4 promoter that is responsive to the transcriptional activator Gal4-VP is provided below

(SEQ ID NO: 94)
GGAGCACTGTCCTCCGAACGTCGGAGCACTGTCCTCCGAACGTCG
GAGCACTGTCCTCCGAACGTCGGAGCACTGTCCTCCGAACGGAGC
ATGTCCTCCGAACGTCGGAGCACTGTCCTCCGAACGACTAGTTAG
GCGTGTACGGTGGGAGGCCTATATAAGCAGAGCTCGTTTAGTGAA
CCGTCAGATCGCCTGGAGACGCCATCCACGCTGTTTTGACCTCCA
TAGAAGACACCGGGACCGATCCAGCCTCTCGACATTGCCGCCACC
Gal4 Promoter (5x)-italics,
Kozak Sequence-boldface,
Minimal CMV promoter-double underline

In some embodiments, the intracellular domain of a P-selectin-specific chimeric receptor polypeptide (e.g., a Notch receptor polypeptide) comprises a transcriptional activator (e.g., Gal4-VP) and when released upon binding of the extracellular domain of the P-selectin-specific chimeric receptor polypeptide (e.g., a Notch receptor polypeptide) to P-selectin, induces production of a CAR in a cell that expresses the P-selectin-specific chimeric receptor polypeptide (e.g., a Notch receptor polypeptide), wherein the CAR is encoded by a nucleic acid that is operably linked to a promoter that is responsive to the transcriptional activator of the released intracellular domain of the P-selectin-specific chimeric receptor polypeptide (e.g., a Notch receptor polypeptide). The CAR in some embodiments comprises a domain that specifically binds an antigen. Examples of such antigens include, e.g., tumor antigens; cancer cell-associated antigens; hematological malignancy antigens; solid tumor antigens; cell surface antigens (e.g., cell surface antigens targeted by a T cell receptor (TCR); intracellular antigens; and the like. Examples of hematological malignancy antigens include, e.g., CD19 (as expressed in e.g., B-cells), CD20 (as expressed in e.g., B-cells), CD22 (as expressed in e.g., B-cells), CD30 (as expressed in e.g., B-cells), CD33 (as expressed in e.g., Myeloid cells), CD70 (as expressed in e.g., B-cell/T-cells), CD123 (as expressed in e.g., Myeloid cells), Kappa (as expressed in e.g., B-cells), Lewis Y (as expressed in e.g., Myeloid cells), NKG2D ligands (as expressed in e.g., Myeloid cells), ROR1 (as expressed in e.g., B-cells), SLAMF7/CS1 (as expressed in e.g., myeloma cells, natural killer cells, T cells, and most B-cell types), CD138 (as expressed in e.g., malignant plasma cells in multiple myelomas), CD56 (as expressed in e.g., myeloma cells, neural cells, natural killer cells, T cells, and trabecular osteoblasts) CD38 (as expressed in e.g., B-cell/T-cells) and CD160 (as expressed in e.g., NK cells/T-cells), and the like. Examples of solid tumor antigens include, e.g., B7H3 (as expressed in e.g., Sarcoma, glioma), CAIX (as expressed in e.g., Kidney), CD44 v6/v7 (as expressed in e.g., Cervical), CD171 (as expressed in e.g., Neuroblastoma), CEA (as expressed in e.g., Colon), EGFRvIII (as expressed in e.g., Glioma), EGP2 (as expressed in e.g., Carcinomas), EGP40 (as expressed in e.g., Colon), EphA2 (as expressed in e.g., Glioma, lung), ErbB2(HER2) (as expressed in e.g., Breast, lung, prostate, glioma), ErbB receptor family (as expressed in e.g., Breast, lung, prostate, glioma), ErbB3/4 (as expressed in e.g., Breast, ovarian), HLA-A1/MAGE1 (as expressed in e.g., Melanoma), HLA-A2/NY-ESO-1 (as expressed in e.g., Sarcoma, melanoma), FR-a (as expressed in e.g., Ovarian), FAPt (as expressed in e.g., Cancer associated fibroblasts), FAR (as expressed in e.g., Rhabdomyosarcoma), GD2 (as expressed in e.g., Neuroblastoma, sarcoma, melanoma), GD3 (as expressed in e.g., Melanoma, lung cancer), HMW-MAA (as expressed in e.g., Melanoma), IL11Ra (as expressed in e.g., Osteosarcoma), IL13Ra2 (as expressed in e.g., Glioma), Lewis Y (as expressed in e.g., Breast/ovarian/pancreatic), Mesothelin (as expressed in e.g., Mesothelioma, breast, pancreas), Muc (as expressed in e.g., Ovarian, breast, prostate), NCAM (as expressed in e.g., Neuroblastoma, colorectal), NKG2D ligands (as expressed in e.g., Ovarian, sacoma), PSCA (as expressed in e.g., Prostate, pancreatic), PSMA (as expressed in e.g., Prostate), TAG72 (as expressed in e.g., Colon), VEGFR-2 (as expressed in e.g., Tumor vasculature), Axl (as expressed in e.g., Lung cancer), Met (as expressed in e.g., Lung cancer), α5β3 (as expressed in e.g., Tumor vasculature), α5β1 (as expressed in e.g., Tumor vasculature), TRAIL-R1/TRAIL-R2 (as expressed in e.g., Solid tumors (colon, lung, pancreas) and hematological malignancies), RANKL (as expressed in e.g., Prostate cancer and bone metastases), Tenacin (as expressed in e.g., Glioma, epithelial tumors (breast, prostate)), EpCAM (as expressed in e.g., Epithelial tumors (breast, colon, lung)), CEA (as expressed in e.g., Epithelial tumors (breast, colon, lung)), gpA33 (as expressed in e.g., Colorectal carcinoma), Mucins (as expressed in e.g., Epithelial tumors (breast, colon, lung, ovarian)), TAG-72 (as expressed in e.g., Epithelial tumors (breast, colon, lung)), EphA3 (as expressed in e.g., Lung, kidney, melanoma, glioma, hematological malignancies) and IGF1R (as expressed in e.g., Lung, breast, head and neck, prostate, thyroid, glioma). Examples of surface and intracellular antigens include, e.g., Her2 (gene symbol ERBB2), MAGE-A1 (gene symbol MAGEA1), MART-1 (gene symbol MLANA), NY-ESO (gene symbol CTAG1), WT1 (gene symbol WT1), MUC17 and MUC13. Examples of other antigens include, e.g., BCMA (gene symbol TNFRSF17), B7H6 (gene symbol NCR3LG1), CAIX (gene symbol CA9), CD123 (gene symbol IL3RA), CD138 (gene symbol SDC1), CD171 (gene symbol L1CAM), CD19 (gene symbol CD19), CD20 (gene symbol CD20), CD22 (gene symbol CD22), CD30 (gene symbol TNFRSF8), CD33 (gene symbol CD33), CD38 (gene symbol CD38), CD44, splice variants incl 7 and 8 (denoted vX in literature) (gene symbol CD44), CEA, CS1 (gene symbol SLAMF7), EGFRvIII (gene symbol EGFR, vIII deletion variant), EGP2, EGP40 (gene symbol EPCAM), Erb family member (gene symbol ERBB1, ERBB2, ERBB3, ERBB4), FAP (gene symbol FAP), fetal acetylcholine receptor (gene symbol AChR), Folate receptor alpha (gene symbol FOLR1), Folate receptor beta (gene symbol FOLR2), GD2, GD3, GPC3 (gene symbol GPC3), Her2/neu (gene symbol ERBB2), IL-13Ra2 (gene symbol IL13RA2), Kappa light chain (gene symbol IGK), Lewis-Y, Mesothelin (gene symbol MSLN), Mucin-1 (gene symbol MUC1), Mucin-16 (gene symbol MUC16), NKG2D ligands, prostate specific membrane antigen (PSMA) (gene symbol FOLH1), prostate stem cell antigen (PSCA) (gene symbol PSCA), receptor tyrosine kinase-like orphan receptor 1 (gene symbol ROR1), and Anaplastic Lymphoma Receptor Tyrosine Kinase (gene symbol ALK).

In some embodiments, the MEAT cells provided herein express at least one chimeric antigen receptor (CAR). CARs are engineered receptors, which graft or confer a specificity of interest onto an immune effector cell. For example, CARs can be used to graft the specificity of a monoclonal antibody onto an immune cell, such as a T cell. In some embodiments, transfer of the coding sequence of the CAR is facilitated by nucleic acid vector, such as a retroviral vector.

There are currently three generations of CARs. In some embodiments, the MEAT cells provided herein express a “first generation” CAR. “First generation” CARs are typically composed of an extracellular antigen binding domain (e.g., a single-chain variable fragment (scFv)) fused to a transmembrane domain fused to cytoplasmic/intracellular domain of the T cell receptor (TCR) chain. “First generation” CARs typically have the intracellular domain from the CD3ζ chain, which is the primary transmitter of signals from endogenous TCRs. “First generation” CARs can provide de novo antigen recognition and cause activation of both CD4+ and CD8+ T cells through their CD3ζ chain signaling domain in a single fusion molecule, independent of HLA-mediated antigen presentation.

In some embodiments, the MEAT cells provided herein express a “second generation” CAR. “Second generation” CARs add intracellular domains from various co-stimulatory molecules (e.g., CD28, 4-1BB, ICOS, OX40) to the cytoplasmic tail of the CAR to provide additional signals to the T cell. “Second generation” CARs comprise those that provide both co-stimulation (e.g., CD28 or 4-1BB) and activation (e.g., CD3ζ). Preclinical studies have indicated that “Second Generation” CARs can improve the antitumor activity of T cells. For example, robust efficacy of “Second Generation” CAR modified T cells was demonstrated in clinical trials targeting the CD19 molecule in patients with chronic lymphoblastic leukemia (CLL) and acute lymphoblastic leukemia (ALL).

In some embodiments, the MEAT cells provided herein express a “third generation” CAR. “Third generation” CARs comprise those that provide multiple co-stimulation (e.g., CD28 and 4-1BB) and activation (e.g., CD3ζ).

In accordance with the presently disclosed subject matter, the CARs of the MEAT cells provided herein comprise an extracellular antigen-binding domain, a transmembrane domain and an intracellular domain. Further, the activity of the MEAT cells can be adjusted by selection of co-stimulatory molecules included in the chimeric antigen receptor.

Extracellular Antigen-Binding Domain of a CAR. In certain embodiments, the extracellular antigen-binding domain of a CAR specifically binds a tumor antigen. In certain embodiments, the extracellular antigen-binding domain is derived from a monoclonal antibody (mAb) that binds to a tumor antigen. In some embodiments, the extracellular antigen-binding domain comprises an scFv. In some embodiments, the extracellular antigen-binding domain comprises a Fab, which is optionally crosslinked. In some embodiments, the extracellular binding domain comprises a F(ab)₂. In some embodiments, any of the foregoing molecules are included in a fusion protein with a heterologous sequence to form the extracellular antigen-binding domain. In certain embodiments, the extracellular antigen-binding domain comprises a human scFv that binds specifically to a tumor antigen. In certain embodiments, the scFv is identified by screening scFv phage library with a tumor antigen-Fc fusion protein.

In certain embodiments, the extracellular antigen-binding domain of a presently disclosed CAR has a high binding specificity and high binding affinity to a tumor antigen. For example, in some embodiments, the extracellular antigen-binding domain of the CAR (embodied, for example, in a human scFv or an analog thereof) binds to a particular tumor antigen with a dissociation constant (K_{d}) of about 1×10⁻⁵ M or less. In certain embodiments, the K_{d} is about 5×10⁻⁶ M or less, about 1×10⁻⁶ M or less, about 5×10⁻⁷ M or less, about 1×10⁻⁷ M or less, about 5×10⁻⁸ M or less, about 1×10⁻⁸ M or less, about 5×10⁻⁹ or less, about 4×10⁻⁹ or less, about 3×10⁻⁹ or less, about 2×10⁻⁹ or less, or about 1×10⁻⁹ M or less. In certain non-limiting embodiments, the K_{d} is from about 3×10⁻⁹ M or less. In certain non-limiting embodiments, the K_{d} is from about 3×10⁻⁹ to about 2×10⁻⁷.

Binding of the extracellular antigen-binding domain (embodiment, for example, in an scFv or an analog thereof) of a presently disclosed tumor antigen-specific CAR can be confirmed by, for example, enzyme-linked immunosorbent assay (ELISA), radioimmunoassay (RIA), FACS analysis, bioassay (e.g., growth inhibition), or Western Blot assay. Each of these assays generally detect the presence of protein-antibody complexes of particular interest by employing a labeled reagent (e.g., an antibody, or an scFv) specific for the complex of interest. For example, the scFv can be radioactively labeled and used in a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a γ counter or a scintillation counter or by autoradiography. In certain embodiments, the extracellular antigen-binding domain of the tumor antigen-specific CAR is labeled with a fluorescent marker. Non-limiting examples of fluorescent markers include green fluorescent protein (GFP), blue fluorescent protein (e.g., EBFP, EBFP2, Azurite, and mKalamal), cyan fluorescent protein (e.g., ECFP, Cerulean, and CyPet), and yellow fluorescent protein (e.g., YFP, Citrine, Venus, and YPet). In certain embodiments, the scFv of a presently disclosed tumor antigen-specific CAR is labeled with GFP.

In some embodiments, the extracellular antigen-binding domain of the expressed CAR binds to a tumor antigen that is expressed by a tumor cell. In some embodiments, the extracellular antigen-binding domain of the expressed CAR binds to a tumor antigen that is expressed on the surface of a tumor cell. In some embodiments, the extracellular antigen-binding domain of the expressed CAR binds to a tumor antigen that is expressed on the surface of a tumor cell in combination with an MHC protein in some embodiments, the MHC protein is a MHC class I protein. In some embodiments, the MHC Class I protein is an HLA-A, HLA-B, or HLA-C molecules. In some embodiments, the extracellular antigen-binding domain of the expressed CAR binds to a tumor antigen that is expressed on the surface of a tumor cell not in combination with an MHC protein.

In some embodiments, the extracellular antigen-binding domain of the expressed CAR binds to a tumor antigen selected from among 5T4, alpha 5pi-integrin, 707-AP, A33, AFP, ART-4, B7H4, BAGE, Bcl-2, β-catenin, Bcr-abl, MN/C IX antibody, CA125, CA19-9, CAMEL, CAP-1, CASP-8, CD4, CD5, CD19, CD20, CD21, CD22, CD25, CDC27/m, CD33, CD37, CD45, CD52, CD56, CD80, CD123, CDK4/m, CEA, c-Met, CS-1, CT, Cyp-B, cyclin B1, DAGE, DAM, EBNA, EGFR, ErbB3, ELF2M, EMMPRIN, EpCam, ephrinB2, estrogen receptor, ETV6-AML1, FAP, ferritin, folate-binding protein, GAGE, G250, GD2, GM1, GM2, GnT-V, gp75, gp100 (Pmel 17), HAGE, HER-2/neu, HLA-A*0201-R170I, HPV E6, HPV E7, Ki-67, HSP70-2M, HST-2, hTERT (or hTRT), iCE, IGF-1R, IL-2R, IL-5, KIAA0205, LAGE, LDLR/FUT, LRP, MAGE, MART, MART-1/melan-A, MART-2/Ski, MC1R, mesothelin, MUC, MUM-1-B, myc, MUM-2, MUM-3, NA88-A, NYESO-1, NY-Eso-B, p53, proteinase-3, p190 minor bcr-abl, Pml/RARa, PRAME, progesterone receptor, PSA, PSM, PSMA, ras, RAGE, RU1 or RU2, RORI, SART-1 or SART-3, survivin, TEL/AML1, TGFp, TPI/m, TRP-1, TRP-2, TRP-2/INT2, tenascin, TSTA tyrosinase, VEGF, and WT1. In certain embodiments, the extracellular antigen-binding domain of the expressed CAR binds to a tumor antigen selected from among CD19 or GD2. Exemplary extracellular antigen-binding domains and methods of generating such domains and associated CARs are described in, e.g., WO2016/191246, WO2017/023859, WO2015/188141, WO2015/070061, WO2012/135854, WO2014/055668, which are incorporated by reference in their entirety, including the sequence listings provided therein.

In certain embodiments, the extracellular antigen-binding domain (e.g., human scFv) comprises a heavy chain variable region and a light chain variable region, optionally linked with a linker sequence, for example a linker peptide (e.g., SEQ NO: 1), between the heavy chain variable region and the light chain variable region. In certain embodiments, the extracellular antigen-binding domain is a human scFv-Fc fusion protein or full length human IgG with VH and VL regions.

In certain embodiments, the extracellular antigen-binding domain comprises a human scFv that binds to a GD2 antigen. In some embodiments, the scFv comprises a polypeptide having an amino acid sequence of SEQ ID NO: 3:

(SEQ ID NO: 3)
MEFGLSWLFLVAILKGVQCGSDILLTQTPLSLPVSLGDQASISCRSSQSLV
HRNGNTYLHWYLQKPGQSPKLLIHKVSNRFSGVPDRFSGSGSGTDFTLKISRVEAE
DLGVYFCSQSTHVPPLTFGAGTKLELKRADAAPTVSIFPGGGGSGGGGSGGGGSGG
GGSEVKLQQSGPSLVEPGASVMISCKASGSSFTGYNMNWVRQNIGKSLEWIGAIDP
YYGGTSYNQKFKGRATLTVDKSSSTAYMHLKSLTSEDSAVYYCVSGMKYWGQGT
SVTVSS.

### Leader Peptide is Underlined; Peptide Linker is Underlined, Linker Peptide is Italicized

In some embodiments, the scFv comprises a polypeptide having an amino acid sequence that is at least 80%, at least 85%, at least 90%, or at least 95% identical to SEQ ID NO: 3. For example, the scFv comprises a polypeptide having an amino acid sequence that is about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 3.

In some embodiments, the scFv is encoded by a nucleic acid having a nucleic acid sequence of SEQ ID NO. 4.

(SEQ ID NO: 4)
TGATAAGGATCTCGAGGCCGGGTAGGGGAGGCGCTTTTCCCAAGGCA
GTCTGGAGCATGCGCTTTAGCAGCCCCGCTGGGCACTTGGCGCTACACAAGTGG
CCTCTGGCCTCGCACACATTCCACATCCACCGGTAGGCGCCAACCGGCTCCGTT
CTTTGGTGGCCCCTTCGCGCCACCTTCTACTCCTCCCCTAGTCAGGAAGTTCCCC
CCCGCCCCGCAGCTCGCGTCGTGCAGGACGTGACAAATGGAAGTAGCACGTCT
CACTAGTCTCGTGCAGATGGACAGCACCGCTGAGCAATGGAAGCGGGTAGGCC
TTTGGGGCAGCGGCCAATAGCAGCTTTGCTCCTTCGCTTTCTGGGCTCAGAGGC
TGGGAAGGGGTGGGTCCGGGGGCGGGCTCAGGGGGGGGCTCAGGGGCGGGGC
GGGCGCCCGAAGGTCCTCCGGAGGCCCGGCATTCTGCACGCTTCAAAAGCGCA
CGTCTGCCGCGCTGTTCTCCTCTTCCTCATCTCCGGGCCTTTCGACCTCC

In some embodiments, the scFv is encoded by a nucleic acid having a nucleic acid sequence that is at least 80%, at least 85%, at least 90%, or at least 95% identical to SEQ ID NO: 4. In some embodiments, the scFv is encoded by a nucleic acid having a nucleic acid sequence of SEQ ID NO: 4. In some embodiments, the scFv is encoded by a nucleic acid having a nucleic acid sequence that is about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 4.

In some embodiments, the anti-GD2 scFv comprises a V_{H} having an amino acid sequence comprising EVKLQQSGPSLVEPGASVMISCKASGSSFTGYNMNWVRQNIGKSLEWIGAIDPYYG GTSYNQKFKGRATLTVDKSSSTAYMHLKSLTSEDSAVYYCVSGMKYWGQGTSVT VSS (SEQ ID NO: 95); and/or a V_{L} having an amino acid sequence comprising

(SEQ ID NO: 96)
DILLTQTPLSLPVSLGDQASISCRSSQSLVHRNGNTYLHWYLQKPGQSP
KLLIHKVSNRFSGVPDRFSGSGSGTDFTLKISRVEAEDLGVYFCSQSTH
VPPLTFGAGTKLELKRADAAPTVSIFP

In certain embodiments, the extracellular antigen-binding domain comprises a human scFv that binds to a CD19 antigen. In some embodiments, the scFv comprises a polypeptide having an amino acid sequence of SEQ ID NO: 97:

(SEQ ID NO: 97)
MALPVTALLLPLALLLHAEVKLQQSGAELVRPGSSVKISCKASGYAFSSY
WMNWVKQRPGQGLEWIGQIYPGDGDTNYNGKFKGQATLTADKSSSTAYMQ
LSGLTSEDSAVYFCARKTISSVVDFYFDYWGQGTTVTVSSGGGGSGGGGS
GGGGSDIELTQSPKFMSTSVGDRVSVTCKASQNVGTNVAWYQQKPGQSPK
PLIYSATYRNSGVPDRFTGSGSGTDFTLTITNVQSKDLADYFCQQYNRYP
YTSGGGTKLEIK

### Leader Peptide is Underlined; Peptide Linker is Underlined, Linker Peptide is Italicized

In some embodiments, the anti-CD19 scFv comprises a V_{H} having an amino acid sequence comprising

EVKLQQSGAELVRPGSSVKISCKASGYAFSSYWMNWVKQRPGQGLEWI GQIYPGDGDTNYNGKFKGQATLTADKSSSTAYMQLSGLTSEDSAVYFCARKTISSV VDFYFDYWGQGTTVTVSS (SEQ ID NO: 98) and/or a V_{L} having an amino acid sequence comprising

(SEQ ID NO: 99)
DIELTQSPKFMSTSVGDRVSVTCKASQNVGTNVAWYQQKPGQSPKPLIY
SATYRNSGVPDRFTGSGSGTDFTLTITNVQSKDLADYFCQQYNRYPYTS
GGGTKLEIK.

In certain non-limiting embodiments, an extracellular antigen-binding domain of the presently disclosed CAR can comprise a linker connecting the heavy chain variable (V_{H}) region and light chain variable (V_{L}) region of the extracellular antigen-binding domain. As used herein, the term “linker” refers to a functional group (e.g., chemical or polypeptide) that covalently attaches two or more polypeptides or nucleic acids so that they are connected to one another. As used herein, a “peptide linker” refers to one or more amino acids used to couple two proteins together (e.g., to couple V_{H} and V_{L} domains). In certain embodiments, the linker comprises amino acids having the sequence set forth in (GGGGS)ₙ, wherein n is 1, 2, 3, 4, 5, 6, 7, 8, 9, 10, 11, 12, 13, 14, or 15 (SEQ ID NO: 5).

Additionally or alternatively, in some embodiments, the extracellular antigen-binding domain can comprise a leader or a signal peptide sequence that directs the nascent protein into the endoplasmic reticulum. The signal peptide or leader can be essential if the CAR is to be glycosylated and anchored in the cell membrane. The signal sequence or leader sequence can be a peptide sequence (about 5, about 10, about 15, about 20, about 25, or about 30 amino acids long) present at the N-terminus of the newly synthesized proteins that direct their entry to the secretory pathway.

In certain embodiments, the signal peptide is covalently joined to the N-terminus of the extracellular antigen-binding domain. In certain embodiments, the signal peptide comprises a human CD8 signal polypeptide comprising amino acids having the sequence set forth in SEQ ID NO: 6 as provided below: MALPVTALLLPLALLLHAARP (SEQ ID NO: 6).

The nucleotide sequence encoding the amino acid sequence of SEQ ID NO: 6 is set forth in SEQ ID NO: 7, which is provided below:

(SEQ ID NO: 7)
ATGGCCCTGCCAGTAACGGCTCTGCTGCTGCCACTTGCTCTGCTCCTCC
ATGCAGCCAGGCCT.

In certain embodiments, the signal peptide comprises a human CD8 signal polypeptide comprising amino acids having the sequence set forth in SEQ ID NO: 8 as provided below: MALPVTALLLPLALLLHA (SEQ ID NO: 8).

The nucleotide sequence encoding the amino acid sequence of SEQ ID NO: 8 is set forth in SEQ ID NO: 9, which is provided below:

(SEQ ID NO: 9)
ATGGCTCTCCCAGTGACTGCCCTACTGCTTCCCCTAGCGCTTCTCCTGC
ATGCA.

In certain embodiments, the signal peptide comprises a signal polypeptide comprising amino acids having the sequence set forth in SEQ ID NO: 10 as provided below: MEFGLSWLFLVAILKGVQCGS (SEQ ID NO: 10).

In certain embodiments, the signal peptide comprises a mouse CD8 signal polypeptide comprising amino acids having the sequence set forth in SEQ ID NO: 11 as provided below: MASPLTRFLSLNLLLLGESII (SEQ ID NO: 11).

The nucleotide sequence encoding the amino acid sequence of SEQ ID NO: 11 is set forth in SEQ ID NO: 12, which is provided below:

(SEQ ID NO: 12)
ATGGCCAGCCCCCTGACCAGGTTCCTGAGCCTGAACCTGCTGCTGCT
GGGCGAGAGCATCATC.

In certain embodiments, the signal peptide comprises a mouse CD8 signal polypeptide comprising amino acids having the sequence set forth in SEQ ID NO: 13 as provided below: MASPLTRFLSLNLLLLGE (SEQ ID NO: 13).

The nucleotide sequence encoding the amino acid sequence of SEQ ID NO: 13 is set forth in SEQ ID NO: 14, which is provided below:

(SEQ ID NO: 14)
ATGGCCAGCCCCCTGACCAGGTTCCTGAGCCTGAACCTGCTGCTGCTGG
GCGAG.

Transmembrane Domain of a CAR. In certain non-limiting embodiments, the transmembrane domain of the CAR comprises a hydrophobic alpha helix that spans at least a portion of the membrane. Different transmembrane domains result in different receptor stability. After antigen recognition, receptors cluster and a signal is transmitted to the cell. In accordance with the presently disclosed subject matter, the transmembrane domain of the CAR can comprise a CD8 polypeptide, a CD28 polypeptide, a CD3ζ polypeptide, a CD4 polypeptide, a 4-1BB polypeptide, an OX40 polypeptide, an ICOS polypeptide, a CTLA-4 polypeptide, a PD-1 polypeptide, a LAG-3 polypeptide, a 2B4 polypeptide, a BTLA polypeptide, a synthetic peptide (e.g., a transmembrane peptide not based on a protein associated with the immune response), or a combination thereof.

In certain embodiments, the transmembrane domain of a presently disclosed CAR comprises a CD28 polypeptide. The CD28 polypeptide can have an amino acid sequence that is at least about 85%, about 90%, about 95%, about 96%, about 97%, about 98%, about 99% or 100% homologous to the sequence having a UniProtKB Reference No: P10747 or NCBI Reference No: NP006130 (SEQ ID NO: 15), or fragments thereof, and/or may optionally comprise up to one or up to two or up to three conservative amino acid substitutions. In certain embodiments, the CD28 polypeptide can have an amino acid sequence that is a consecutive portion of SEQ ID NO: 15 which is at least 20, or at least 30, or at least 40, or at least 50, and up to 220 amino acids in length. Additionally or alternatively, in non-limiting various embodiments, the CD28 polypeptide has an amino acid sequence of amino acids 1 to 220, 1 to 50, 50 to 100, 100 to 150, 114 to 220, 150 to 200, or 200 to 220 of SEQ ID NO: 15. In certain embodiments, the CAR of the present disclosure comprises a transmembrane domain comprising a CD28 polypeptide, and optionally an intracellular domain comprising a co-stimulatory signaling region that comprises a CD28 polypeptide. In certain embodiments, the CD28 polypeptide comprised in the transmembrane domain and the intracellular domain has an amino acid sequence of amino acids 114 to 220 of SEQ ID NO: 15. In certain embodiments, the CD28 polypeptide comprised in the transmembrane domain has an amino acid sequence of amino acids 153 to 179 of SEQ ID NO: 15.

SEQ ID NO: 15 is provided below:

(SEQ ID NO: 15)
MLRLLLALNLFPSIQVTGNKILVKQSPMLVAYDNAVNLSCKYSYNLFSR
EFRASLHKGLDSAVEVCVVYGNYSQQLQVYSKTGFNCDGKLGNESVTFY
LQNLYVNQTDIYFCKIEVMYPPPYLDNEKSNGTIIHVKGKHLCPSPLFP
GPSKPFWVLVVVGGVLACYSLLVTVAFIIFWVRSKRSRLLHSDYMNMTP
RRPGPTRKHYQPYAPPRDFAAYRS

In accordance with the presently disclosed subject matter, a “CD28 nucleic acid molecule” refers to a polynucleotide encoding a CD28 polypeptide. In certain embodiments, the CD28 nucleic acid molecule encoding the CD28 polypeptide comprised in the transmembrane domain (and optionally the intracellular domain (e.g., the co-stimulatory signaling region)) of the presently disclosed CAR (e.g., amino acids 114 to 220 of SEQ ID NO: 15 or amino acids 153 to 179 of SEQ ID NO: 15) comprises at least a portion of the sequence set forth in SEQ ID NO: 16 as provided below.

(SEQ ID NO: 16)
attgaagttatgtatcctcctccttacctagacaatgagaagagcaatggaaccattatccatgtgaaagggaaacacctttgtccaagt
cccctatttcccggaccttctaagcccttttgggtgctggtggtggttggtggagtcctggcttgctatagcttgctagtaacagtggcct
ttattattttctgggtgaggagtaagaggagcaggctcctgcacagtgactacatgaacatgactccccgccgccccgggcccaccc
gcaagcattaccagccctatgccccaccacgcgacttcgcagcctatcgctcc

In certain embodiments, the transmembrane domain comprises a CD8 polypeptide. The CD8 polypeptide can have an amino acid sequence that is at least about 85%, about 90%, about 95%, about 96%, about 97%, about 98%, about 99% or about 100%) homologous to SEQ ID NO: 17 (homology herein may be determined using standard software such as BLAST or FASTA) as provided below, or fragments thereof, and/or may optionally comprise up to one or up to two or up to three conservative amino acid substitutions. In certain embodiments, the CD8 polypeptide can have an amino acid sequence that is a consecutive portion of SEQ ID NO: 17 which is at least 20, or at least 30, or at least 40, or at least 50, and up to 235 amino acids in length. Additionally or alternatively, in various embodiments, the CD8 polypeptide has an amino acid sequence of amino acids 1 to 235, 1 to 50, 50 to 100, 100 to 150, 150 to 200, or 200 to 235 of SEQ ID NO: 17.

(SEQ ID NO: 17)
MALPVTALLLPLALLLHAARPSQFRVSPLDRTWNLGETVELKCQVLLSN
PTSGCSWLFQPRGAAASPTFLLYLSQNKPKAAEGLDTQRFSGKRLGDTFVLTLSDF
RRENEGYYFCSALSNSIMYFSHFVPVFLPAKPTTTPAPRPPTPAPTIASQPLSLRPEAC
RPAAGGAVHTRGLDFACDIYIWAPLAGTCGVLLLSLVITLYCNHRNRRRVCKCPRP
WKSGDKPSLSARYV

In certain embodiments, the transmembrane domain comprises a CD8 polypeptide comprising amino acids having the sequence set forth in SEQ ID NO: 18 as provided below:

(SEQ ID NO: 18)
PTTTPAPRPPTPAPTIASQPLSLRPEACRPAAGGAVHTRGLDFACD
IYIWAPLAGTCGVLLLSLVITLYCN

In accordance with the presently disclosed subject matter, a “CD8 nucleic acid molecule” refers to a polynucleotide encoding a CD8 polypeptide. In certain embodiments, the CD8 nucleic acid molecule encoding the CD8 polypeptide comprised in the transmembrane domain of the presently disclosed CAR (SEQ ID NO: 18) comprises nucleic acids having the sequence set forth in SEQ ID NO: 19 as provided below.

(SEQ ID NO: 19)
CCCACCACGACGCCAGCGCCGCGACCACCAACCCCGGCGCCCACGAT
CGCGTCGCAGCCCCTGTCCCTGCGCCCAGAGGCGTGCCGGCCAGCGGCG
GGGGGCGCAGTGCACACGAGGGGGCTGGACTTCGCCTGTGATATCTACA
TCTGGGCGCCCCTGGCCGGGACTTGTGGGGTCCTTCTCCTGTCACTGGT
TATCACCCTTTACTGCAAC

In certain non-limiting embodiments, a CAR can also comprise a spacer region that links the extracellular antigen-binding domain to the transmembrane domain. The spacer region can be flexible enough to allow the antigen-binding domain to orient in different directions to facilitate antigen recognition while preserving the activating activity of the CAR. In certain non-limiting embodiments, the spacer region can be the hinge region from IgG1, the CH₂CH₃ region of immunoglobulin and portions of CD3, a portion of a CD28 polypeptide (e.g., SEQ ID NO: 15), a portion of a CD8 polypeptide (e.g., SEQ ID NO: 17), a variation of any of the foregoing which is at least about 80%, at least about 85%, at least about 90%, or at least about 95% homologous thereto, or a synthetic spacer sequence. In certain non-limiting embodiments, the spacer region may have a length between about 1-50 (e.g., 5-25, 10-30, or 30-50) amino acids.

Intracellular Domain of a CAR. In certain non-limiting embodiments, an intracellular domain of the CAR can comprise a CD3ζ polypeptide, which can activate or stimulate a cell (e.g., a cell of the lymphoid lineage, e.g., a T cell). CD3ζ comprises 3 ITAMs, and transmits an activation signal to the cell (e.g., a cell of the lymphoid lineage, e.g., a T cell) after antigen is bound. The CD3ζ polypeptide can have an amino acid sequence that is at least about 85%, about 90%, about 95%, about 96%, about 97%, about 98%, about 99% or about 100% homologous to the sequence having a NCBI Reference No: NP_932170 (SEQ ID NO: 20), or fragments thereof, and/or may optionally comprise up to one or up to two or up to three conservative amino acid substitutions.

In certain embodiments, the CD3ζ polypeptide can have an amino acid sequence that is a consecutive portion of SEQ ID NO: 21 which is at least 20, or at least 30, or at least 40, or at least 50, and up to 164 amino acids in length. Additionally or alternatively, in various embodiments, the CD3ζ polypeptide has an amino acid sequence of amino acids 1 to 164, 1 to 50, 50 to 100, 100 to 150, or 150 to 164 of SEQ ID NO: 21. In certain embodiments, the CD3ζ polypeptide has an amino acid sequence of amino acids 52 to 164 of SEQ ID NO: 21.

SEQ ID NO: 21 is provided below:

(SEQ ID NO: 21)
MKWKALFTAAILQAQLPITEAQSFGLLDPKLCYLLDGILFIYGVILTAL
FLRVKFSRSADAPAYQQGQNQLYNELNLGRREEYDVLDKRRGRDPEMGG
KPQRRKNPQEGLYNELQKDKMAEAYSEIGMKGERRRGKGHDGLYQGLST
ATKDTYDALHMQALPPR

In certain embodiments, the CD3ζ polypeptide has the amino acid sequence set forth in SEQ ID NO: 22, which is provided below:

(SEQ ID NO: 22)
RVKFSRSAEPPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR

In certain embodiments, the CD3ζ polypeptide has the amino acid sequence set forth in SEQ ID NO: 23, which is provided below:

(SEQ ID NO: 23)
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR

In accordance with the presently disclosed subject matter, a “CD3ζ nucleic acid molecule” refers to a polynucleotide encoding a CD3ζ polypeptide. In certain embodiments, the CD3ζ nucleic acid molecule encoding the CD3ζ polypeptide (SEQ ID NO: 22) comprised in the intracellular domain of the presently disclosed CAR comprises a nucleotide sequence as set forth in SEQ ID NO: 24 as provided below.

(SEQ ID NO: 24)
AGAGTGAAGTTCAGCAGGAGCGCAGAGCCCCCCGCGTACCAGCAGGGCC
AGAACCAGCTCTATAACGAGCTCAATCTAGGACGAAGAGAGGAGTACGA
TGTTTTGGACAAGAGACGTGGCCGGGACCCTGAGATGGGGGGAAAGCCG
AGAAGGAAGAACCCTCAGGAAGGCCTGTACAATGAACTGCAGAAAGATA
AGATGGCGGAGGCCTACAGTGAGATTGGGATGAAAGGCGAGCGCCGGAG
GGGCAAGGGGCACGATGGCCTTTACCAGGGTCTCAGTACAGCCACCAAG
GACACCTACGACGCCCTTCACATGCAGGCCCTGCCCCCTCGCG

In certain embodiments, the CD3ζ nucleic acid molecule encoding the CD3ζ polypeptide (SEQ ID NO: 23) comprised in the intracellular domain of the presently disclosed CAR comprises a nucleotide sequence as set forth in SEQ ID NO: 25 as provided below.

(SEQ ID NO: 25)
AGAGTGAAGTTCAGCAGGAGCGCAGACGCCCCCGCGTACCAGCAGGGCC
AGAACCAGCTCTATAACGAGCTCAATCTAGGACGAAGAGAGGAGTACGA
TGTTTTGGACAAGAGACGTGGCCGGGACCCTGAGATGGGGGGAAAGCCG
AGAAGGAAGAACCCTCAGGAAGGCCTGTACAATGAACTGCAGAAAGAT
AAGATGGCGGAGGCCTACAGTGAGATTGGGATGAAAGGCGAGCGCCGGA
GGGGCAAGGGGCACGATGGCCTTTACCAGGGTCTCAGTACAGCCACCAA
GGACACCTACGACGCCCTTCACATGCAGGCCCTGCCCCCTCGCTAA

In certain non-limiting embodiments, an intracellular domain of the CAR further comprises at least one signaling region. The at least one signaling region can include a CD28 polypeptide, a 4-1BB polypeptide, an OX40 polypeptide, an ICOS polypeptide, a DAP-10 polypeptide, a PD-1 polypeptide, a CTLA-4 polypeptide, a LAG-3 polypeptide, a 2B4 polypeptide, a BTLA polypeptide, a synthetic peptide (not based on a protein associated with the immune response), or a combination thereof.

In certain embodiments, the signaling region is a co-stimulatory signaling region.

In certain embodiments, the co-stimulatory signaling region comprises at least one co-stimulatory molecule, which can provide optimal lymphocyte activation. As used herein, “co-stimulatory molecules” refer to cell surface molecules other than antigen receptors or their ligands that are required for an efficient response of lymphocytes to antigen. The at least one co-stimulatory signaling region can include a CD28 polypeptide, a 4-1BB polypeptide, an OX40 polypeptide, an ICOS polypeptide, a DAP-10 polypeptide, or a combination thereof. The co-stimulatory molecule can bind to a co-stimulatory ligand, which is a protein expressed on cell surface that upon binding to its receptor produces a co-stimulatory response, i.e., an intracellular response that effects the stimulation provided when an antigen binds to its CAR molecule. Co-stimulatory ligands, include, but are not limited to CD80, CD86, CD70, OX40L, 4-1BBL, CD48, TNFRSF14, and PD-L1. As one example, a 4-1BB ligand (i.e., 4-1BBL) may bind to 4-1BB (also known as “CD 137”) for providing an intracellular signal that in combination with a CAR signal induces an effector cell function of the CAR⁺ T cell. CARs comprising an intracellular domain that comprises a co-stimulatory signaling region comprising 4-1BB, ICOS or DAP-10 are disclosed in U.S. Pat. No. 7,446,190, which is herein incorporated by reference in its entirety. In certain embodiments, the intracellular domain of the CAR comprises a co-stimulatory signaling region that comprises a CD28 polypeptide. In certain embodiments, the intracellular domain of the CAR comprises a co-stimulatory signaling region that comprises two co-stimulatory molecules: CD28 and 4-1BB or CD28 and OX40.

4-1BB can act as a tumor necrosis factor (TNF) ligand and have stimulatory activity. The 4-1BB polypeptide can have an amino acid sequence that is at least about 85%, about 90%, about 95%, about 96%, about 97%, about 98%, about 99% or 100% homologous to the sequence having a UniProtKB Reference No: P41273 or NCBI Reference No: NP_001552 (SEQ ID NO: 26) or fragments thereof, and/or may optionally comprise up to one or up to two or up to three conservative amino acid substitutions.

SEQ ID NO: 26 is provided below:

(SEQ ID NO: 26)
MEYASDASLDPEAPWPPAPRARACRVLPWALVAGLLLLLLLAAACAVFLACPWA
VSGARASPGSAASPRLREGPELSPDDPAGLLDLRQGMFAQLVAQNVLLIDGPLSWY
SDPGLAGVSLTGGLSYKEDTKELVVAKAGVYYVFFQLELRRVVAGEGSGSVSLAL
HLQPLRSAAGAAALALTVDLPPASSEARNSAFGFQGRLLHLSAGQRLGVHLHTEAR
ARHAWQLTQGATVLGLFRVTPEIPAGLPSPRSE

In certain embodiments, the 4-1BB co-stimulatory domain has the amino acid sequence set forth in SEQ ID NO: 27, which is provided below:

(SEQ ID NO: 27)
KRGRKKLLYIFKQPFMRPVQTTQEEDGCSCRFPEEEEGGCEL

In accordance with the presently disclosed subject matter, a “4-1BB nucleic acid molecule” refers to a polynucleotide encoding a 4-1BB polypeptide. In certain embodiments, the 4-1BB nucleic acid molecule encoding the 4-1BB polypeptide (SEQ ID NO: 27) comprised in the intracellular domain of the presently disclosed CAR comprises a nucleotide sequence as set forth in SEQ ID NO: 28 as provided below.

(SEQ ID NO: 28)
AAACGGGGCAGAAAGAAGCTCCTGTATATATTCAAACAACCATTTATGA
GACCAGTACAAACTACTCAAGAGGAAGATGGCTGTAGCTGCCGATTTCC
AGAAGAAGAAGAAGGAGGATGTGAACTG

An OX40 polypeptide can have an amino acid sequence that is at least about 85%, about 90%, about 95%, about 96%, about 97%, about 98%, about 99% or 100% homologous to the sequence having a UniProtKB Reference No: P43489 or NCBI Reference No: NP_003318 (SEQ ID NO: 29), or fragments thereof, and/or may optionally comprise up to one or up to two or up to three conservative amino acid substitutions.

SEQ ID NO: 29 is provided below:

(SEQ ID NO: 29)
MCVGARRLGRGPCAALLLLGLGLSTVTGLHCVGDTYPSNDRCCHECRPGNGMVS
RCSRSQNTVCRPCGPGFYNDVVSSKPCKPCTWCNLRSGSERKQLCTATQDTVCRC
RAGTQPLDSYKPGVDCAPCPPGHFSPGDNQACKPWTNCTLAGKHTLQPASNSSDAI
CEDRDPPATQPQETQGPPARPITVQPTEAWPRTSQGPSTRPVEVPGGRAVAAILGLG
LVLGLLGPLAILLALYLLRRDQRLPPDAHKPPGGGSFRTPIQEEQADAHSTLAKI

In accordance with the presently disclosed subject matter, an “OX40 nucleic acid molecule” refers to a polynucleotide encoding an OX40 polypeptide.

An ICOS polypeptide can have an amino acid sequence that is at least about 85%, about 90%, about 95%, about 96%, about 97%, about 98%, about 99% or 100% homologous to the sequence having a NCBI Reference No: NP_036224 (SEQ ID NO: 30) or fragments thereof, and/or may optionally comprise up to one or up to two or up to three conservative amino acid substitutions.

SEQ ID NO: 30 is provided below:

(SEQ ID NO: 30)
MKSGLWYFFLFCLRIKVLTGEINGSANYEMFIFHNGGVQILCKYPDIVQQFKMQLL
KGGQILCDLTKTKGSGNTVSIKSLKFCHSQLSNNSVSFFLYNLDHSHANYYFCNLSI
FDPPPFKVTLTGGYLHIYESQLCCQLKFWLPIGCAAFVVVCILGCILICWLTKKKYSS
SVHDPNGEYMFMRAVNTAKKSRLTDVTL

In accordance with the presently disclosed subject matter, an “ICOS nucleic acid molecule” refers to a polynucleotide encoding an ICOS polypeptide.

CTLA-4 is an inhibitory receptor expressed by activated T cells, which when engaged by its corresponding ligands (CD80 and CD86; B7-1 and B7-2, respectively), mediates activated T cell inhibition or anergy. In both preclinical and clinical studies, CTLA-4 blockade by systemic antibody infusion, enhanced the endogenous anti-tumor response albeit, in the clinical setting, with significant unforeseen toxicities.

CTLA-4 contains an extracellular V domain, a transmembrane domain, and a cytoplasmic tail. Alternate splice variants, encoding different isoforms, have been characterized. The membrane-bound isoform functions as a homodimer interconnected by a disulfide bond, while the soluble isoform functions as a monomer. The intracellular domain is similar to that of CD28, in that it has no intrinsic catalytic activity and contains one YVKM motif (SEQ ID NO: 31) able to bind PI3K, PP2A and SHP-2 and one proline-rich motif able to bind SH3 containing proteins. One role of CTLA-4 in inhibiting T cell responses seem to be directly via SHP-2 and PP2A dephosphorylation of TCR-proximal signaling proteins such as CD3 and LAT. CTLA-4 can also affect signaling indirectly via competing with CD28 for CD80/86 binding. CTLA-4 has also been shown to bind and/or interact with PI3K, CD80, AP2M1, and PPP2R5A.

In accordance with the presently disclosed subject matter, a CTLA-4 polypeptide can have an amino acid sequence that is at least about 85%, about 90%, about 95%, about 96%, about 97%, about 98%, about 99% or about 100% homologous to UniProtKB/Swiss-Prot Ref. No.: P16410.3 (SEQ ID NO: 32) (homology herein may be determined using standard software such as BLAST or FASTA) or fragments thereof, and/or may optionally comprise up to one or up to two or up to three conservative amino acid substitutions.

SEQ ID NO: 32 is provided below:

(SEQ ID NO: 32)
MACLGFQRHKAQLNLATRTWPCTLLFFLLFIPVFCKAMHVAQPAVVLASSRGIASF
VCEYASPGKATEVRVTVLRQADSQVTEVCAATYMMGNELTFLDDSICTGTSSGNQ
VNLTIQGLRAMDTGLYICKVELMYPPPYYLGIGNGTQIYVIDPEPCPDSDFLLWILA
AVSSGLFFYSFLLTAVSLSKMLKKRSPLTTGVYVKMPPTEPECEKQFQPYFIPIN

In accordance with the presently disclosed subject matter, a “CTLA-4 nucleic acid molecule” refers to a polynucleotide encoding a CTLA-4 polypeptide.

PD-1 is a negative immune regulator of activated T cells upon engagement with its corresponding ligands PD-L1 and PD-L2 expressed on endogenous macrophages and dendritic cells. PD-1 is a type I membrane protein of 268 amino acids. PD-1 has two ligands, PD-L1 and PD-L2, which are members of the B7 family. The protein's structure comprises an extracellular IgV domain followed by a transmembrane region and an intracellular tail. The intracellular tail contains two phosphorylation sites located in an immunoreceptor tyrosine-based inhibitory motif and an immunoreceptor tyrosine-based switch motif, that PD-1 negatively regulates TCR signals. SHP-I and SHP-2 phosphatases bind to the cytoplasmic tail of PD-1 upon ligand binding. Upregulation of PD-L1 is one mechanism tumor cells may evade the host immune system. In pre-clinical and clinical trials, PD-1 blockade by antagonistic antibodies induced anti-tumor responses mediated through the host endogenous immune system. In accordance with the presently disclosed subject matter, a PD-1 polypeptide can have an amino acid sequence that is at least about 85%, about 90%, about 95%, about 96%, about 97%, about 98%, about 99% or about 100% homologous to NCBI Reference No: NP_005009.2 (SEQ ID NO: 33) or fragments thereof, and/or may optionally comprise up to one or up to two or up to three conservative amino acid substitutions.

SEQ ID NO: 33 is provided below:

(SEQ ID NO: 33)
MQIPQAPWPVVWAVLQLGWRPGWFLDSPDRPWNPPTFSPALLVVTEGDNATFTCS
FSNTSESFVLNWYRMSPSNQTDKLAAFPEDRSQPGQDCRFRVTQLPNGRDFHMSV
VRARRNDSGTYLCGAISLAPKAQIKESLRAELRVTERRAEVPTAHPSPSPRPAGQFQ
TLVVGVVGGLLGSLVLLVWVLAVICSRAARGTIGARRTGQPLKEDPSAVPVFSVDY
GELDFQWREKTPEPPVPCVPEQTEYATIVFPSGMGTSSPARRGSADGPRSAQPLRPE
DGHCSWPL

In accordance with the presently disclosed subject matter, a “PD-1 nucleic acid molecule” refers to a polynucleotide encoding a PD-1 polypeptide.

Lymphocyte-activation protein 3 (LAG-3) is a negative immune regulator of immune cells. LAG-3 belongs to the immunoglobulin (Ig) superfamily and contains 4 extracellular Ig-like domains. The LAG3 gene contains 8 exons. The sequence data, exon/intron organization, and chromosomal localization all indicate a close relationship of LAG3 to CD4. LAG3 has also been designated CD223 (cluster of differentiation 223).

In accordance with the presently disclosed subject matter, a LAG-3 polypeptide can have an amino acid sequence that is at least about 85%, about 90%, about 95%, about 96%, about 97%, about 98%, about 99% or about 100% homologous to UniProtKB/Swiss-Prot Ref. No.: P18627.5 (SEQ ID NO: 34) or fragments thereof, and/or may optionally comprise up to one or up to two or up to three conservative amino acid substitutions.

SEQ ID NO: 34 is provided below:

(SEQ ID NO: 34)
MWEAQFLGLLFLQPLWVAPVKPLQPGAEVPVVWAQEGAPAQLPCSPTIPLQDLSLL
RRAGVTWQHQPDSGPPAAAPGHPLAPGPHPAAPSSWGPRPRRYTVLSVGPGGLRS
GRLPLQPRVQLDERGRQRGDFSLWLRPARRADAGEYRAAVHLRDRALSCRLRLRL
GQASMTASPPGSLRASDWVILNCSFSRPDRPASVHWFRNRGQGRVPVRESPHHHLA
ESFLFLPQVSPMDSGPWGCILTYRDGFNVSIMYNLTVLGLEPPTPLTVYAGAGSRVG
LPCRLPAGVGTRSFLTAKWTPPGGGPDLLVTGDNGDFTLRLEDVSQAQAGTYTCHI
HLQEQQLNATVTLAIITVTPKSFGSPGSLGKLLCEVTPVSGQERFVWSSLDTPSQRSF
SGPWLEAQEAQLLSQPWQCQLYQGERLLGAAVYFTELSSPGAQRSGRAPGALPAG
HLLLFLILGVLSLLLLVTGAFGFHLWRRQWRPRRFSALEQGIHPPQAQSKIEELEQEP
EPEPEPEPEPEPEPEPEQL

In accordance with the presently disclosed subject matter, a “LAG-3 nucleic acid molecule” refers to a polynucleotide encoding a LAG-3 polypeptide.

Natural Killer Cell Receptor 2B4 (2B4) mediates non-MHC restricted cell killing on NK cells and subsets of T cells. To date, the function of 2B4 is still under investigation, with the 2B4-S isoform believed to be an activating receptor, and the 2B4-L isoform believed to be a negative immune regulator of immune cells. 2B4 becomes engaged upon binding its high-affinity ligand, CD48. 2B4 contains a tyrosine-based switch motif, a molecular switch that allows the protein to associate with various phosphatases. 2B4 has also been designated CD244 (cluster of differentiation 244).

In accordance with the presently disclosed subject matter, a 2B4 polypeptide can have an amino acid sequence that is at least about 85%, about 90%, about 95%, about 96%, about 97%, about 98%, about 99% or about 100% homologous to UniProtKB/Swiss-Prot Ref. No.: Q9BZW8.2 (SEQ ID NO: 35) or fragments thereof, and/or may optionally comprise up to one or up to two or up to three conservative amino acid substitutions.

SEQ ID NO: 35 is provided below:

(SEQ ID NO: 35)
MLGQVVTLILLLLLKVYQGKGCQGSADHVVSISGVPLQLQPNSIQTKVDSIAWKKL
LPSQNGFHHILKWENGSLPSNTSNDRFSFIVKNLSLLIKAAQQQDSGLYCLEVTSISG
KVQTATFQVFVFESLLPDKVEKPRLQGQGKILDRGRCQVALSCLVSRDGNVSYAW
YRGSKLIQTAGNLTYLDEEVDINGTHTYTCNVSNPVSWESHTLNLTQDCQNAHQEF
RFWPFLVIIVILSALFLGTLACFCVWRRKRKEKQSETSPKEFLTIYEDVKDLKTRRNH
EQEQTFPGGGSTIYSMIQSQSSAPTSQEPAYTLYSLIQPSRKSGSRKRNHSPSFNSTIY
EVIGKSQPKAQNPARLSRKELENFDVYS

In accordance with the presently disclosed subject matter, a “2B4 nucleic acid molecule” refers to a polynucleotide encoding a 2B4 polypeptide.

B- and T-lymphocyte attenuator (BTLA) expression is induced during activation of T cells, and BTLA remains expressed on Th1 cells but not Th2 cells. Like PD1 and CTLA4, BTLA interacts with a B7 homolog, B7H4. However, unlike PD-1 and CTLA-4, BTLA displays T-Cell inhibition via interaction with tumor necrosis family receptors (TNF-R), not just the B7 family of cell surface receptors. BTLA is a ligand for tumor necrosis factor (receptor) superfamily, member 14 (TNFRSF14), also known as herpes virus entry mediator (HVEM). BTLA-HVEM complexes negatively regulate T-cell immune responses. BTLA activation has been shown to inhibit the function of human CD8⁺ cancer-specific T cells. BTLA has also been designated as CD272 (cluster of differentiation 272).

In accordance with the presently disclosed subject matter, a BTLA polypeptide can have an amino acid sequence that is at least about 85%>, about 90%, about 95%, about 96%, about 97%, about 98%, about 99% or about 100% homologous to UniProtKB/Swiss-Prot Ref. No.: Q7Z6A9.3 (SEQ ID NO: 36) or fragments thereof, and/or may optionally comprise up to one or up to two or up to three conservative amino acid substitutions.

SEQ ID NO: 36 is provided below:

(SEQ ID NO: 36)
MKTLPAMLGTGKLFWVFFLIPYLDIWNIHGKESCDVQLYIKRQSEHSILAGDPFELE
CPVKYCANRPHVTWCKLNGTTCVKLEDRQTSWKEEKNISFFILHFEPVLPNDNGSY
RCSANFQSNLIESHSTTLYVTDVKSASERPSKDEMASRPWLLYRLLPLGGLPLLITTC
FCLFCCLRRHQGKQNELSDTAGREINLVDAHLKSEQTEASTRQNSQVLLSETGIYD
NDPDLCFRMQEGSEVYSNPCLEENKPGIVYASLNHSVIGPNSRLARNVKEAPTEYA
SICVRS

In accordance with the presently disclosed subject matter, a “BTLA nucleic acid molecule” refers to a polynucleotide encoding a BTLA polypeptide.

### Polynucleotides, Polypeptides and Analogs

Also included in the presently disclosed subject matter are polypeptides including P-selectin-specific chimeric receptor polypeptides (e.g., a Notch receptor polypeptides) comprising an extracellular domain that specifically binds to a P-selectin polypeptide (e.g., a human or mouse P-selectin) (e.g., an anti-P-selectin scFv (e.g., a human scFv), a Fab, or a (Fab)₂), PSGL-1), antigen-specific CARs, CD3ζ, CD8, CD28, etc. or fragments thereof, and polynucleotides encoding the same, that are modified in ways that enhance their biological activity when expressed in a MEAT cell. The presently disclosed subject matter provides methods for optimizing an amino acid sequence or a nucleic acid sequence by producing an alteration in the sequence. Such alterations may comprise certain mutations, deletions, insertions, or post-translational modifications. The presently disclosed subject matter further comprises analogs of any naturally-occurring polypeptide of the presently disclosed subject matter. Analogs can differ from a naturally-occurring polypeptide of the presently disclosed subject matter by amino acid sequence differences, by post-translational modifications, or by both. Analogs of the presently disclosed subject matter can generally exhibit at least about 85%, about 90%, about 91%, about 92%, about 93%, about 94%, about 95%, about 96%, about 97%, about 98%, about 99% or more identity or homology with all or part of a naturally-occurring amino acid sequence of the presently disclosed subject matter. The length of sequence comparison is at least about 5, about 10, about 15, about 20, about 25, about 50, about 75, about 100 or more amino acid residues. Again, in an exemplary approach to determining the degree of identity, a BLAST program may be used, with a probability score between e⁻³ and e⁻¹⁰° indicating a closely related sequence. Modifications comprise in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring polypeptides of the presently disclosed subject matter by alterations in primary sequence. These include genetic variants, both natural and induced (for example, resulting from random mutagenesis by irradiation or exposure to ethanemethyl sulfate or by site-specific mutagenesis as described in Sambrook, Fritsch and Maniatis, Molecular Cloning: A Laboratory Manual (2nd ed.), CSH Press, 1989, or Ausubel et al., supra). Also included are cyclized peptides, molecules, and analogs which contain residues other than L-amino acids, e.g., D-amino acids or non-naturally occurring or synthetic amino acids, e.g., beta (β) or gamma (γ) amino acids.

In addition to full-length polypeptides, the presently disclosed subject matter also provides fragments of any one of the polypeptides or peptide domains of the presently disclosed subject matter. A fragment can be at least about 5, about 10, about 13, or about 15 amino acids. In some embodiments, a fragment is at least about 20 contiguous amino acids, at least about 30 contiguous amino acids, or at least about 50 contiguous amino acids. In some embodiments, a fragment is at least about 60 to about 80, about 100, about 200, about 300 or more contiguous amino acids. Fragments of the presently disclosed subject matter can be generated by methods known to those of ordinary skill in the art or may result from normal protein processing (e.g., removal of amino acids from the nascent polypeptide that are not required for biological activity or removal of amino acids by alternative mRNA splicing or alternative protein processing events).

Non-protein analogs have a chemical structure designed to mimic the functional activity of a protein of the present technology. Such analogs are administered according to methods of the presently disclosed subject matter. Such analogs may exceed the physiological activity of the original polypeptide. Methods of analog design are well known in the art, and synthesis of analogs can be carried out according to such methods by modifying the chemical structures such that the resultant analogs increase the antineoplastic activity of the original polypeptide when expressed in a MEAT cell. These chemical modifications include, but are not limited to, substituting alternative R groups and varying the degree of saturation at specific carbon atoms of a reference polypeptide. The protein analogs can be relatively resistant to in vivo degradation, resulting in a more prolonged therapeutic effect upon administration. Assays for measuring functional activity include, but are not limited to, those described in the Examples below.

In accordance with the presently disclosed subject matter, the polynucleotides encoding a P-selectin-specific chimeric receptor polypeptide (e.g., a Notch receptor polypeptide) comprising an extracellular domain that specifically binds to a P-selectin polypeptide (e.g., a human or mouse P-selectin) (e.g., an anti-P-selectin scFv (e.g., a human scFv), a Fab, or a (Fab)₂), PSGL-1), an antigen-specific CAR, CD3ζ, CD8, CD28, etc. can be modified by codon optimization. Codon optimization can alter both naturally occurring and recombinant gene sequences to achieve the highest possible levels of productivity in any given expression system. Factors that are involved in different stages of protein expression include codon adaptability, mRNA structure, and various cis-elements in transcription and translation. Any suitable codon optimization methods or technologies that are known to ones skilled in the art can be used to modify the polynucleotides of the presently disclosed subject matter, including, but not limited to, OptimumGene™, Encor optimization, and Blue Heron.

### Vectors

Many expression vectors are available and known to those of skill in the art and can be used for expression of polypeptides provided herein. The choice of expression vector will be influenced by the choice of host expression system. Such selection is well within the level of skill of the skilled artisan. In general, expression vectors can include transcriptional promoters and optionally enhancers, translational signals, and transcriptional and translational termination signals. Expression vectors that are used for stable transformation typically have a selectable marker which allows selection and maintenance of the transformed cells. In some cases, an origin of replication can be used to amplify the copy number of the vector in the cells.

Vectors also can contain additional nucleotide sequences operably linked to the ligated nucleic acid molecule, such as, for example, an epitope tag such as for localization, e.g., a hexa-his tag (SEQ ID NO: 101) or a myc tag, hemagglutinin tag or a tag for purification, for example, a GST fusion, and a sequence for directing protein secretion and/or membrane association.

Expression of any of the P-selectin-specific chimeric receptor polypeptides (e.g., a Notch receptor polypeptide) can be controlled by any promoter/enhancer known in the art. Suitable bacterial promoters are well known in the art and described herein below. Other suitable promoters for mammalian cells, yeast cells and insect cells are well known in the art and some are exemplified below. Selection of the promoter used to direct expression of a heterologous nucleic acid depends on the particular application and is within the level of skill of the skilled artisan. Promoters which can be used include but are not limited to eukaryotic expression vectors containing the SV40 early promoter (Bernoist and Chambon, Nature 290:304-310(1981)), the promoter contained in the 3′ long terminal repeat of Rous sarcoma virus (Yamamoto et al., Cell 22:787-797(1980)), the herpes thymidine kinase promoter (Wagner et al., Proc. Natd. Acad. Sci. USA 75: 1441-1445 (1981)), the regulatory sequences of the metallothionein gene (Brinster et al., Nature 296:39-42 (1982)); prokaryotic expression vectors such as the β-lactamase promoter (Jay et al., Proc. Natl. Acad. Sci. USA 75:5543 (1981)) or the tac promoter (DeBoer et al., Proc. Natl. Acad. Sci. USA 50:21-25(1983)); see also “Useful Proteins from Recombinant Bacteria”: in Scientific American 242:79-94 (1980)); plant expression vectors containing the nopaline synthetase promoter (Herrera-Estrella et al., Nature 505:209-213(1984)) or the cauliflower mosaic virus 35S RNA promoter (Gardner et al., Nucleic Acids Res. 9:2871(1981)), and the promoter of the photosynthetic enzyme ribulose bisphosphate carboxylase (Herrera-Estrella et al., Nature 510: 1 15-120(1984)); promoter elements from yeast and other fungi such as the Gal4 promoter, the alcohol dehydrogenase promoter, the phosphoglycerol kinase promoter, the alkaline phosphatase promoter, and the following animal transcriptional control regions that exhibit tissue specificity and have been used in transgenic animals: elastase I gene control region which is active in pancreatic acinar cells (Swift et al., Cell 55:639-646 (1984); Ornitz et al., Cold Spring Harbor Symp. Quant. Biol. 50:399-409(1986); MacDonald, Hepatology 7:425-515 (1987)); insulin gene control region which is active in pancreatic beta cells (Hanahan et al., Nature 515: 115-122 (1985)), immunoglobulin gene control region which is active in lymphoid cells (Grosschedl et al., Cell 55:647-658 (1984); Adams et al., Nature 515:533-538 (1985); Alexander et al., Mol. Cell Biol. 7: 1436-1444 (1987)), mouse mammary tumor virus control region which is active in testicular, breast, lymphoid and mast cells (Leder et al., Cell 15:485-495 (1986)), albumin gene control region which is active in liver (Pinckert et al., Genes and Devel. 1:268-276 (1987)), alpha-fetoprotein gene control region which is active in liver (Krumlauf et al., Mol. Cell. Biol. 5:1639-403 (1985)); Hammer et al., Science 255:53-58 (1987)), alpha-1 antitrypsin gene control region which is active in liver (Kelsey et al., Genes and Devel. 7:161-171 (1987)), beta globin gene control region which is active in myeloid cells (Magram et al., Nature 515:338-340 (1985)); Kollias et al., Cell 5:89-94 (1986)), myelin basic protein gene control region which is active in oligodendrocyte cells of the brain (Readhead et al., Cell 15:703-712 (1987)), myosin light chain-2 gene control region which is active in skeletal muscle (Shani, Nature 514:283-286 (1985)), and gonadotrophic releasing hormone gene control region which is active in gonadotrophs of the hypothalamus (Mason et al., Science 254: 1372-1378 (1986)).

In addition to the promoter, the expression vector typically contains a transcription unit or expression cassette that contains all the additional elements required for the expression of a P-selectin-specific chimeric receptor polypeptide (e.g., a Notch receptor polypeptide) in host cells. A typical expression cassette contains a promoter operably linked to the nucleic acid sequence encoding the polypeptide chains of interest and signals required for efficient polyadenylation of the transcript, ribosome binding sites and translation termination. Additional elements of the cassette can include enhancers. In addition, the cassette typically contains a transcription termination region downstream of the structural gene to provide for efficient termination. The termination region can be obtained from the same gene as the promoter sequence or can be obtained from different genes.

Some expression systems have markers that provide gene amplification such as thymidine kinase and dihydrofolate reductase. Alternatively, high yield expression systems not involving gene amplification are also suitable, such as using a baculovirus vector in insect cells, with a nucleic acid sequence encoding a germline antibody chain under the direction of the polyhedron promoter or other strong baculovirus promoter.

Any methods known to those of skill in the art for the insertion of DNA fragments into a vector can be used to construct expression vectors containing a nucleic acid encoding any of the polypeptides provided herein. These methods can include in vitro recombinant DNA and synthetic techniques and in vivo recombinants (genetic recombination). The insertion into a cloning vector can, for example, be accomplished by ligating the DNA fragment into a cloning vector which has complementary cohesive termini. If the complementary restriction sites used to fragment the DNA are not present in the cloning vector, the ends of the DNA molecules can be enzymatically modified. Alternatively, any site desired can be produced by ligating nucleotide sequences (linkers) onto the DNA termini; these ligated linkers can contain specific chemically synthesized nucleic acids encoding restriction endonuclease recognition sequences.

Exemplary plasmid vectors useful to produce the polypeptides provided herein contain a strong promoter, such as the HCMV immediate early enhancer/promoter or the MHC class I promoter, an intron to enhance processing of the transcript, such as the HCMV immediate early gene intron A, and a polyadenylation (poly A) signal, such as the late SV40 polyA signal.

Genetic modification of MEAT cells can be accomplished by transducing a substantially homogeneous cell composition with a recombinant DNA or RNA construct. The vector can be a retroviral vector (e.g., gamma retroviral), which is employed for the introduction of the DNA or RNA construct into the host cell genome. For example, a polynucleotide encoding a P-selectin-specific chimeric receptor polypeptide (e.g., a Notch receptor polypeptide) described herein or a CAR polypeptide described herein can be cloned into a retroviral vector and expression can be driven from its endogenous promoter, from the retroviral long terminal repeat, or from an alternative internal promoter.

Non-viral vectors or RNA may be used as well. Random chromosomal integration, or targeted integration (e.g., using a nuclease, transcription activator-like effector nucleases (TALENs), Zinc-finger nucleases (ZFNs), and/or clustered regularly interspaced short palindromic repeats (CRISPRs), or transgene expression (e.g., using a natural or chemically modified RNA) can be used.

For initial genetic modification of the cells to provide P-selectin-specific chimeric receptor polypeptide (e.g., a Notch receptor polypeptide) and/or CAR polypeptide expressing cells, a retroviral vector is generally employed for transduction, however any other suitable viral vector or non-viral delivery system can be used. For subsequent genetic modification of the cells to provide cells comprising an antigen presenting complex comprising at least two co-stimulatory ligands, retroviral gene transfer (transduction) likewise proves effective. Combinations of retroviral vector and an appropriate packaging line are also suitable, where the capsid proteins will be functional for infecting human cells. Various amphotropic virus-producing cell lines are known, including, but not limited to, PA12 (Miller, et al., Mol. Cell. Biol. 5:431-437 (1985)); PA317 (Miller, et al., Mol. Cell. Biol. 6:2895-2902 (1986)); and CRIP (Danos, et al. Proc. Natl. Acad. Sci. USA 85:6460-6464 (1988)). Non-amphotropic particles are suitable too, e.g., particles pseudotyped with VSVG, RD 114 or GALV envelope and any other known in the art.

Possible methods of transduction also include direct co-culture of the cells with producer cells, e.g., by the method of Bregni, et al., Blood 80: 1418-1422(1992), or culturing with viral supernatant alone or concentrated vector stocks with or without appropriate growth factors and polycations, e.g., by the method of Xu, et al., Exp. Hemat. 22:223-230 (1994); and Hughes, et al., J. Clin. Invest. 89: 1817 (1992).

Transducing viral vectors can be used to express a co-stimulatory ligand and/or secretes a cytokine (e.g., 4-1BBL and/or IL-12) in a MEAT cell. In some embodiments, the chosen vector exhibits high efficiency of infection and stable integration and expression (see, e.g., Cayouette et al., Human Gene Therapy 8:423-430 (1997); Kido et al., Current Eye Research 15:833-844 (1996); Bloomer et al., Journal of Virology 71:6641-6649, 1997; Naldini et al., Science 272:263 267 (1996); and Miyoshi et al., Proc. Natd. Acad. Sci. U.S.A. 94: 10319, (1997)). Other viral vectors that can be used include, for example, adenoviral, lentiviral, and adeno-associated viral vectors, vaccinia virus, a bovine papilloma virus, or a herpes virus, such as Epstein-Barr Virus (also see, for example, the vectors of Miller, Human Gene Therapy 15-14, (1990); Friedman, Science 244: 1275-1281 (1989); Eglitis et al., BioTechniques 6:608-614, (1988); Tolstoshev et al., Current Opinion in Biotechnology 1:55-61(1990); Sharp, The Lancet 337: 1277-1278 (1991); Cornetta et al., Nucleic Acid Research and Molecular Biology 36:311-322 (1987); Anderson, Science 226:401-409 (1984); Moen, Blood Cells 17:407-416 (1991); Miller et al., Biotechnology 7:980-990 (1989); Le Gal La Salle et al., Science 259:988-990 (1993); and Johnson, Chest 107:77S-83S (1995)). Retroviral vectors are particularly well developed and have been used in clinical settings (Rosenberg et al., N. Engl. J. Med 323:370 (1990); Anderson et al., U.S. Pat. No. 5,399,346).

In certain non-limiting embodiments, the vector expressing a presently disclosed P-selectin-specific chimeric receptor polypeptide (e.g., a Notch receptor polypeptide) and/or a presently disclosed CAR polypeptide is a retroviral vector, e.g., an oncoretroviral vector. In some instances, the retroviral vector is a SFG retroviral vector or murine stem cell virus (MSCV) retroviral vector. In certain non-limiting embodiments, the vector expressing a presently disclosed P-selectin-specific chimeric receptor polypeptide (e.g., a Notch receptor polypeptide) and/or a presently disclosed CAR polypeptide is a lentiviral vector or a transposon vector.

Non-viral approaches can also be employed for the expression of a protein in a cell. For example, a nucleic acid molecule can be introduced into a cell by administering the nucleic acid in the presence of lipofection (Feigner et al., Proc. Nat'l. Acad. Sci. U.S.A. 84:7413, (1987); Ono et al., Neuroscience Letters 17:259 (1990); Brigham et al., Am. J. Med. Sci. 298:278, (1989); Staubinger et al., Methods in Enzymology 101:512 (1983)), asialoorosomucoid-polylysine conjugation (Wu et al., Journal of Biological Chemistry 263: 14621 (1988); Wu et al., Journal of Biological Chemistry 264: 16985 (1989)), or by micro-injection under surgical conditions (Wolff et al., Science 247: 1465 (1990)). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g., Zinc finger nucleases, meganucleases, or TALE nucleases). Transient expression may be obtained by RNA electroporation.

cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g., the elongation factor 1a enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

The resulting cells can be grown under conditions similar to those for unmodified cells, whereby the modified cells can be expanded and used for a variety of purposes.

### MEAT Cells

The MEAT cells of the presently disclosed subject matter can be any type of T cells, including, but not limited to, T helper cells, cytotoxic T cells, memory T cells (including central memory T cells, stem-cell-like memory T cells (or stem-like memory T cells), and two types of effector memory T cells: e.g., T_{EM} cells and TEMRA cells, Regulatory T cells (also known as suppressor T cells), Natural killer T cells, Mucosal associated invariant T cells, and γδ T cells. T cells are lymphocytes that mature in the thymus and are chiefly responsible for cell-mediated immunity. T cells are involved in the adaptive immune system. Cytotoxic T cells (CTL or killer T cells) are a subset of T lymphocytes capable of inducing the death of infected somatic or tumor cells.

The MEAT cells of the presently disclosed subject matter express (1) a P-selectin-specific chimeric receptor polypeptide comprising (a) an extracellular domain that specifically binds P-selectin; (b) a heterologous receptor polypeptide comprising one or more ligand-inducible proteolytic cleavage sites (e.g., Notch receptor polypeptide); and (c) an intracellular domain comprising a transcriptional activator (e.g., Gal4-VP16), wherein binding of the extracellular domain to P-selectin induces cleavage of the heterologous receptor polypeptide (e.g., Notch receptor polypeptide) at the one or more ligand-inducible proteolytic cleavage sites to release the intracellular domain, and (2) a CAR polypeptide, wherein the CAR polypeptide is encoded by a nucleic acid that is operably linked to a promoter that is responsive to the transcriptional activator of the released intracellular domain of the P-selectin-specific chimeric receptor polypeptide. In some embodiments of the MEAT cells disclosed herein, the CAR polypeptide specifically bind and inhibit a tumor antigen for the treatment of cancer, e.g., for treatment of solid tumor. Such MEAT cells can be administered to a subject (e.g., a human subject) in need thereof for the treatment of cancer. In certain embodiments, the MEAT cell can be a CD4⁺ T cell or a CD8⁺ T cell. In certain embodiments, the MEAT cell is a CD4⁺ T cell. In certain embodiments, the MEAT cell is a CD8⁺ T cell.

The presently disclosed MEAT cells of the present technology may further include at least one recombinant or exogenous co-stimulatory ligand. For example, the presently disclosed MEAT cells can be further transduced with at least one co-stimulatory ligand, such that the MEAT cells co-express or are induced to co-express a P-selectin-specific chimeric receptor polypeptide (e.g., a Notch receptor polypeptide) described herein, a CAR polypeptide described herein, and the at least one co-stimulatory ligand. Co-stimulatory ligands include, but are not limited to, members of the tumor necrosis factor (TNF) superfamily, and immunoglobulin (Ig) superfamily ligands. TNF is a cytokine involved in systemic inflammation and stimulates the acute phase reaction. Its primary role is in the regulation of immune cells. Members of TNF superfamily share a number of common features. The majority of TNF superfamily members are synthesized as type II transmembrane proteins (extracellular C-terminus) containing a short cytoplasmic segment and a relatively long extracellular region. TNF superfamily members include, without limitation, nerve growth factor (NGF), CD40L (CD40L)/CD 154, CD137L/4-1BBL, TNF-a, CD134L/OX40L/CD252, CD27L/CD70, Fas ligand (FasL), CD30L/CD153, tumor necrosis factor beta (TNFP)/lymphotoxin-alpha (LTa), lymphotoxin-beta O-Tβ), CD257/B cell-activating factor (B AFF)/Bly s/THANK/Tall-1, glucocorticoid-induced TNF Receptor ligand (GITRL), and T F-related apoptosis-inducing ligand (TRAIL), LIGHT (TNFSF14). The immunoglobulin (Ig) superfamily is a large group of cell surface and soluble proteins that are involved in the recognition, binding, or adhesion processes of cells. These proteins share structural features with immunoglobulins they possess an immunoglobulin domain (fold). Immunoglobulin superfamily ligands include, but are not limited to, CD80 and CD86, both ligands for CD28, PD-L1/(B7-H1) that ligands for PD-1. In certain embodiments, the at least one co-stimulatory ligand is selected from the group consisting of 4-1BBL, CD80, CD86, CD70, OX40L, CD48, TNFRSF14, PD-L1, and combinations thereof.

Furthermore, the presently disclosed MEAT cells can further comprise at least one exogenous cytokine. For example, a presently disclosed MEAT cell can be further transduced with at least one cytokine, such that the MEAT secrete the at least one cytokine as well as express a P-selectin-specific chimeric receptor polypeptide (e.g., a Notch receptor polypeptide) disclosed herein, and a CAR polypeptide disclosed herein. In certain embodiments, the at least one cytokine is selected from the group consisting of IL-2, IL-3, IL-6, IL-7, IL-11, IL-12, IL-15, IL-17, and IL-21. In certain embodiments, the cytokine is IL-12.

The MEAT cells can be generated from peripheral donor lymphocytes, e.g., those disclosed in Sadelain, M., et al., Nat Rev Cancer 3:35-45 (2003), in Morgan, R. A. et al. (2006) Science 314: 126-129, in Panelli et al. (2000) J Immunol 164:495-504; Panelli et al. (2000) J Immunol 164:4382-4392 (2000), and in Dupont et al. (2005) Cancer Res 65:5417-5427; Papanicolaou et al. (2003) Blood 102:2498-2505. The MEAT cells (e.g., T cells) can be autologous, non-autologous (e.g., allogeneic), or derived in vitro from engineered progenitor or stem cells.

The unpurified source of T cells can be any known in the art, such as the bone marrow, fetal, neonate or adult or other hematopoietic cell source, e.g., fetal liver, peripheral blood or umbilical cord blood. Various techniques can be employed to separate the cells. For instance, negative selection methods can remove non-immune cells and non-T cells initially. Monoclonal antibodies are particularly useful for identifying markers associated with particular cell lineages and/or stages of differentiation for both positive and negative selections.

A large proportion of terminally differentiated cells can be initially removed by a relatively crude separation. For example, magnetic bead separations can be used initially to remove large numbers of irrelevant cells. In some embodiments, at least about 80%, usually at least 70% of the total hematopoietic cells will be removed prior to cell isolation.

Procedures for separation include, but are not limited to, density gradient centrifugation; resetting; coupling to particles that modify cell density; magnetic separation with antibody-coated magnetic beads; affinity chromatography; cytotoxic agents joined to or used in conjunction with a mAb, including, but not limited to, complement and cytotoxins; and panning with antibody attached to a solid matrix, e.g., plate, chip, elutriation or any other convenient technique.

Techniques for separation and analysis include, but are not limited to, flow cytometry, which can have varying degrees of sophistication, e.g., a plurality of color channels, low angle and obtuse light scattering detecting channels, impedance channels.

The cells can be selected against dead cells, by employing dyes associated with dead cells such as propidium iodide (PI). In some embodiments, the cells are collected in a medium comprising 2% fetal calf serum (FCS) or 0.2% bovine serum albumin (BSA) or any other suitable, preferably sterile, isotonic medium.

In some embodiments, the MEAT cells may comprise one or more additional modifications. For example, in some embodiments, the MEAT cells comprise and express (is transduced to express) a chimeric co-stimulatory receptor (CCR). CCR is described in Krause et al. (1998) J Exp. Med. 188(4):619-626, and US20020018783, the contents of which are incorporated by reference in their entireties. CCRs mimic co-stimulatory signals, but do not provide a T-cell activation signal, e.g., CCRs lack a CD3ζ polypeptide. CCRs provide co-stimulation, e.g., a CD28-like signal, in the absence of the natural co-stimulatory ligand on the antigen-presenting cell. A combinatorial antigen recognition, i.e., use of a CCR in combination with an engineered receptor (e.g., CAR), can augment T-cell reactivity against the dual-antigen expressing T cells, thereby improving selective tumor targeting.

In some embodiments, the MEAT cells are further modified to suppress expression of one or more genes. In some embodiments, the MEAT cells are further modified via genome editing. Various methods and compositions for targeted cleavage of genomic DNA have been described. Such targeted cleavage events can be used, for example, to induce targeted mutagenesis, induce targeted deletions of cellular DNA sequences, and facilitate targeted recombination at a predetermined chromosomal locus. See, for example, U.S. Pat. Nos. 7,888,121; 7,972,854; 7,914,796; 7,951,925; 8,110,379; 8,409,861; 8,586,526; U.S. Patent Publications 20030232410; 20050208489; 20050026157; 20050064474; 20060063231; 201000218264; 20120017290; 20110265198; 20130137104; 20130122591; 20130177983 and 20130177960, the disclosures of which are incorporated by reference in their entireties. These methods often involve the use of engineered cleavage systems to induce a double strand break (DSB) or a nick in a target DNA sequence such that repair of the break by an error born process such as non-homologous end joining (NHEJ) or repair using a repair template (homology directed repair or HDR) can result in the knock out of a gene or the insertion of a sequence of interest (targeted integration). Cleavage can occur through the use of specific nucleases such as engineered zinc finger nucleases (ZFN), transcription-activator like effector nucleases (TALENs), or using the CRISPR/Cas system with an engineered crRNA/tracr RNA (‘single guide RNA’) to guide specific cleavage. In some embodiments, the MEAT cells are modified to disrupt or reduce expression of an endogenous T-cell receptor gene (see, e.g. WO 2014153470, which is incorporated by reference in its entirety). In some embodiments, the MEAT cells are modified to result in disruption or inhibition of PD1, PDL-1 or CTLA-4 (see, e.g. U.S. Patent Publication 20140120622), or other immunosuppressive factors known in the art (Wu et al. (2015) Oncoimmunology 4(7): e1016700, Mahoney et al. (2015) Nature Reviews Drug Discovery 14, 561-584).

### Administration

The MEAT cells of the presently disclosed subject matter can be provided systemically or directly to a subject for treating cancer. In certain embodiments, MEAT cells are directly injected into an organ of interest. Additionally or alternatively, the MEAT cells are provided indirectly to the organ of interest, for example, by administration into the circulatory system (e.g., the tumor vasculature) or into the tissue of interest (e.g., solid tumor). Expansion and differentiation agents can be provided prior to, during or after administration of cells and compositions to increase production of the MEAT cells either in vitro or in vivo.

MEAT cells of the presently disclosed subject matter can be administered in any physiologically acceptable vehicle, systemically or regionally, normally intravascularly, intraperitoneally, intrathecally, or intrapleurally, although they may also be introduced into bone or other convenient site where the cells may find an appropriate site for regeneration and differentiation (e.g., thymus). In certain embodiments, at least 1×10⁵ cells can be administered, eventually reaching 1×10¹⁰ or more. In certain embodiments, at least 1×10⁶ cells can be administered. A cell population comprising MEAT cells can comprise a purified population of cells. Those skilled in the art can readily determine the percentage of MEAT cells in a cell population using various well-known methods, such as fluorescence activated cell sorting (FACS). The ranges of purity in cell populations comprising MEAT cells can be from about 50% to about 55%, from about 55% to about 60%, about 60% to about 65%, from about 65% to about 70%, from about 70% to about 75%, from about 75% to about 80%, from about 80% to about 85%; from about 85% to about 90%, from about 90% to about 95%, or from about 95 to about 100%. Dosages can be readily adjusted by those skilled in the art (e.g., a decrease in purity may require an increase in dosage). The MEAT cells can be introduced by injection, catheter, or the like. If desired, factors can also be included, including, but not limited to, interleukins, e.g., IL-2, IL-3, IL 6, IL-11, IL-7, IL-12, IL-15, IL-21, as well as the other interleukins, the colony stimulating factors, such as G-, M- and GM-CSF, interferons, e.g., γ-interferon.

In certain embodiments, compositions of the presently disclosed subject matter comprise pharmaceutical compositions comprising MEAT cells expressing (1) a P-selectin-specific chimeric receptor polypeptide comprising (a) an extracellular domain that specifically binds P-selectin; (b) a heterologous receptor polypeptide comprising one or more ligand-inducible proteolytic cleavage sites (e.g., Notch receptor polypeptide); and (c) an intracellular domain comprising a transcriptional activator (e.g., Gal4-VP16), wherein binding of the extracellular domain to P-selectin induces cleavage of the heterologous receptor polypeptide (e.g., Notch receptor polypeptide) at the one or more ligand-inducible proteolytic cleavage sites to release the intracellular domain, and (2) a CAR polypeptide, wherein the CAR polypeptide is encoded by a nucleic acid that is operably linked to a promoter that is responsive to the transcriptional activator of the released intracellular domain of the P-selectin-specific chimeric receptor polypeptide, with a pharmaceutically acceptable carrier.

Administration can be autologous or non-autologous. For example, MEAT cells and compositions comprising the same can be obtained from one subject, and administered to the same subject or a different, compatible subject. Peripheral blood derived T cells of the presently disclosed subject matter or their progeny (e.g., in vivo, ex vivo or in vitro derived) can be administered via localized injection, including catheter administration, systemic injection, localized injection, intravenous injection, or parenteral administration. When administering a pharmaceutical composition of the presently disclosed subject matter (e.g., a pharmaceutical composition comprising any and all embodiments of MEAT cells), it can be formulated in a unit dosage injectable form (solution, suspension, emulsion).

### Formulations

MEAT cells expressing (1) a P-selectin-specific chimeric receptor polypeptide comprising (a) an extracellular domain that specifically binds P-selectin; (b) a heterologous receptor polypeptide comprising one or more ligand-inducible proteolytic cleavage sites (e.g., Notch receptor polypeptide); and (c) an intracellular domain comprising a transcriptional activator (e.g., Gal4-VP16), wherein binding of the extracellular domain to P-selectin induces cleavage of the heterologous receptor polypeptide (e.g., Notch receptor polypeptide) at the one or more ligand-inducible proteolytic cleavage sites to release the intracellular domain, and (2) a CAR polypeptide, wherein the CAR polypeptide is encoded by a nucleic acid that is operably linked to a promoter that is responsive to the transcriptional activator of the released intracellular domain of the P-selectin-specific chimeric receptor polypeptide, and compositions comprising the same can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the compositions of the presently disclosed subject matter, e.g., a composition comprising MEAT cells, in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the presently disclosed subject matter, however, any vehicle, diluent, or additive used would have to be compatible with the MEAT cells of the presently disclosed subject matter.

The compositions can be isotonic, i.e., they can have the same osmotic pressure as blood and lacrimal fluid. The desired isotonicity of the compositions of the presently disclosed subject matter may be accomplished using sodium chloride, or other pharmaceutically acceptable agents such as dextrose, boric acid, sodium tartrate, propylene glycol or other inorganic or organic solutes. Sodium chloride is suitable particularly for buffers containing sodium ions.

Viscosity of the compositions, if desired, can be maintained at the selected level using a pharmaceutically acceptable thickening agent. Methylcellulose can be used because it is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Those skilled in the art will recognize that the components of the compositions should be selected to be chemically inert and will not affect the viability or efficacy of the MEAT cells as described in the presently disclosed subject matter. This will present no problem to those skilled in chemical and pharmaceutical principles, or problems can be readily avoided by reference to standard texts or by simple experiments (not involving undue experimentation), from this disclosure and the documents cited herein.

One consideration concerning the therapeutic use of the MEAT cells of the presently disclosed subject matter is the quantity of cells necessary to achieve an optimal effect. The quantity of cells to be administered will vary for the subject being treated. In certain embodiments, from about 10² to about 10¹², from about 10′ to about 10¹¹, from about 10⁴ to about 10¹⁰, from about 10⁵ to about 10⁹, or from about 10⁶ to about 10⁸ MEAT cells of the presently disclosed subject matter are administered to a subject. More effective cells may be administered in even smaller numbers. In some embodiments, at least about 1×10⁸, about 2×10⁸, about 3×10⁸, about 4×10⁸, about 5×10⁸, about 1×10⁹, about 5×10⁹, about 1×10¹⁰, about 5×10¹⁰, about 1×10¹¹, about 5×10¹¹, about 1×10¹² or more MEAT cells of the presently disclosed subject matter are administered to a human subject. The precise determination of what would be considered an effective dose may be based on factors individual to each subject, including their size, age, sex, weight, and condition of the particular subject. Dosages can be readily ascertained by those skilled in the art from this disclosure and the knowledge in the art. Generally, MEAT cells are administered at doses that are nontoxic or tolerable to the patient.

The skilled artisan can readily determine the amount of cells and optional additives, vehicles, and/or carrier in compositions to be administered in methods of the presently disclosed subject matter. Typically, any additives (in addition to the active cell(s) and/or agent(s)) are present in an amount of from about 0.001% to about 50% by weight) solution in phosphate buffered saline, and the active ingredient is present in the order of micrograms to milligrams, such as from about 0.0001 wt % to about 5 wt %, from about 0.0001 wt % to about 1 wt %, from about 0.0001 wt % to about 0.05 wt %, from about 0.001 wt % to about 20 wt %, from about 0.01 wt % to about 10 wt %, or from about 0.05 wt % to about 5 wt %. For any composition to be administered to an animal or human, and for any particular method of administration, toxicity should be determined, such as by determining the lethal dose (LD) and LD50 in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

### Therapeutic Uses of the MEAT Cells of the Present Technology

For treatment, the amount of the MEAT cells provided herein administered is an amount effective in producing the desired effect, for example, treatment of a cancer or one or more symptoms of a cancer. An effective amount can be provided in one or a series of administrations of the MEAT cells provided herein. An effective amount can be provided in a bolus or by continuous perfusion. For adoptive immunotherapy using antigen-specific T cells, cell doses in the range of about 10⁶ to about 10¹⁰ are typically infused. The MEAT cells of the presently disclosed subject matter can be administered by any methods known in the art, including, but not limited to, pleural administration, intravenous administration, subcutaneous administration, intranodal administration, intratumoral administration, intrathecal administration, intrapleural administration, intraperitoneal administration, and direct administration to the thymus. In certain embodiments, the MEAT cells and the compositions comprising thereof are intravenously administered to the subject in need. Methods for administering cells for adoptive cell therapies, including, for example, donor lymphocyte infusion and engineered T cell therapies, and regimens for administration are known in the art and can be employed for administration of the MEAT cells provided herein.

The presently disclosed subject matter provides various methods of using the MEAT cells provided herein, expressing (1) a P-selectin-specific chimeric receptor polypeptide comprising (a) an extracellular domain that specifically binds P-selectin; (b) a heterologous receptor polypeptide comprising one or more ligand-inducible proteolytic cleavage sites (e.g., Notch receptor polypeptide); and (c) an intracellular domain, wherein binding of the extracellular domain to P-selectin induces cleavage of the heterologous receptor polypeptide (e.g., Notch receptor polypeptide) at the one or more ligand-inducible proteolytic cleavage sites to release the intracellular domain, and (2) a CAR polypeptide, wherein the CAR polypeptide is encoded by a nucleic acid that is operably linked to a promoter that is responsive to the transcriptional activator of the released intracellular domain of the P-selectin-specific chimeric receptor polypeptide. For example, the presently disclosed subject matter provides methods of reducing tumor burden in a subject. In one non-limiting example, the method of reducing tumor burden comprises administering an effective amount of the presently disclosed MEAT cells to the subject, thereby inducing tumor cell death in the subject.

The presently disclosed MEAT cells can reduce the number of tumor cells, reduce tumor size, and/or eradicate the tumor in the subject. In certain embodiments, the method of reducing tumor burden comprises administering an effective amount of MEAT cells to the subject, thereby inducing tumor cell death in the subject. Non-limiting examples of suitable tumors include adrenal cancers, bladder cancers, blood cancers, bone cancers, brain cancers, breast cancers, carcinoma, cervical cancers, colon cancers, colorectal cancers, corpus uterine cancers, ear, nose and throat (ENT) cancers, endometrial cancers, esophageal cancers, gastrointestinal cancers, glioma, head and neck cancers, Hodgkin's disease, intestinal cancers, kidney cancers, larynx cancers, acute and chronic leukemias, liver cancers, lymph node cancers, lymphomas, lung cancers, medulloblastoma, melanomas, mesothelioma, myelomas, nasopharynx cancers, neuroblastomas, non-Hodgkin's lymphoma, oral cancers, ovarian cancers, pancreatic cancers, penile cancers, pharynx cancers, prostate cancers, rectal cancers, retinoblastomas, sarcomas (e.g., osteosarcoma, soft tissue sarcoma, Ewing's sarcoma, rhabdomyosarcoma, liposarcoma, fibrosarcoma, malignant fibrous histiocytoma, leiomyosarcoma, and spindle cell sarcoma), seminomas, skin cancers, stomach cancers, teratomas, testicular cancers, thyroid cancers, uterine cancers, vaginal cancers, vascular tumors, and metastases thereof. In some embodiments, the cancer is a relapsed or refractory cancer. In some embodiments, the cancer is resistant to one or more cancer therapies, e.g., one or more chemotherapeutic drugs.

The presently disclosed subject matter also provides methods of increasing or lengthening survival of a subject having a neoplasia (e.g., a tumor). In one non-limiting example, the method of increasing or lengthening survival of a subject having neoplasia (e.g., a tumor) comprises administering an effective amount of the presently disclosed MEAT cell to the subject, thereby increasing or lengthening survival of the subject. The presently disclosed subject matter further provides methods for treating or preventing a neoplasia (e.g., a tumor) in a subject, comprising administering the presently disclosed MEAT cells to the subject.

Cancers whose growth can be inhibited using the MEAT cells of the presently disclosed subject matter comprise cancers typically responsive to immunotherapy. Non-limiting examples of cancers for treatment include multiple myeloma, neuroblastoma, glioma, acute myeloid leukemia, colon cancer, pancreatic cancer, thyroid cancer, small cell lung cancer, and NK cell lymphoma.

Additionally, the presently disclosed subject matter provides methods of increasing immune-activating cytokine production in response to a cancer cell or virally infected cell in a subject. In one non-limiting example, the method comprises administering the presently disclosed MEAT cell to the subject. The immune-activating cytokine can be granulocyte macrophage colony stimulating factor (GM-CSF), IFNα, IFN-β, IFN-γ, TNF-a, IL-2, IL-3, IL-6, IL-1 1, IL-7, IL-12, IL-15, IL-21, interferon regulatory factor 7 (IRF7), and combinations thereof.

Suitable human subjects for therapy typically comprise two treatment groups that can be distinguished by clinical criteria. Subjects with “advanced disease” or “high tumor burden” are those who bear a clinically measurable tumor (e.g., multiple myeloma). A clinically measurable tumor is one that can be detected on the basis of tumor mass (e.g., by palpation, CAT scan, sonogram, mammogram or X-ray; positive biochemical or histopathologic markers on their own are insufficient to identify this population). A pharmaceutical composition embodied in the presently disclosed subject matter is administered to these subjects to elicit an anti-tumor response, with the objective of palliating their condition. Ideally, reduction in tumor mass occurs as a result, but any clinical improvement constitutes a benefit. Clinical improvement comprises decreased risk or rate of progression or reduction in pathological consequences of the tumor (e.g., multiple myeloma).

A second group of suitable subjects is known in the art as the “adjuvant group.” These are individuals who have had a history of neoplasia (e.g., multiple myeloma), but have been responsive to another mode of therapy. The prior therapy can have included, but is not restricted to, surgical resection, radiotherapy, and traditional chemotherapy. As a result, these individuals have no clinically measurable tumor. However, they are suspected of being at risk for progression of the disease, either near the original tumor site, or by metastases. This group can be further subdivided into high-risk and low-risk individuals. The subdivision is made on the basis of features observed before or after the initial treatment. These features are known in the clinical arts, and are suitably defined for each different neoplasia. Features typical of high-risk subgroups are those in which the tumor (e.g., multiple myeloma) has invaded neighboring tissues, or who show involvement of lymph nodes. Another group has a genetic predisposition to neoplasia (e.g., multiple myeloma) but has not yet evidenced clinical signs of neoplasia (e.g., multiple myeloma). For instance, women testing positive for a genetic mutation associated with breast cancer, but still of childbearing age, can wish to receive one or more of the compositions described herein in treatment prophylactically to prevent the occurrence of neoplasia until it is suitable to perform preventive surgery.

The subjects can have an advanced form of disease (e.g., multiple myeloma), in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

Further modification can be introduced to the MEAT cells disclosed herein to avert or minimize the risks of immunological complications (known as “malignant T-cell transformation”), e.g., graft versus-host disease (GvHD), or when healthy tissues express the same target antigens as the tumor cells, leading to outcomes similar to GvHD. One exemplary modification includes engineering a suicide gene into any and all embodiments of the MEAT cells disclosed herein. Suitable suicide genes include, but are not limited to, Herpes simplex virus thymidine kinase (hsv-tk), inducible Caspase 9 Suicide gene (iCasp-9), and a truncated human epidermal growth factor receptor (EGFRt) polypeptide. In certain embodiments, the suicide gene is an EGFRt polypeptide. The EGFRt polypeptide can enable T cell elimination by administering anti-EGFR monoclonal antibody (e.g., cetuximab). The suicide gene can be included within the vector comprising nucleic acids encoding any embodiment of the P-selectin specific chimeric receptor polypeptide (e.g., a Notch receptor polypeptide) disclosed herein, and/or any embodiment of the CAR polypeptide disclosed herein.

A presently disclosed MEAT cell incorporated with a suicide gene can be pre-emptively eliminated at a given time point post T cell infusion, or eradicated at the earliest signs of toxicity.

### Combination Therapy

The MEAT cells of the present technology may be employed in conjunction with other therapeutic agents useful in the treatment of cancers. For example, the MEAT cells of the present technology may be separately, sequentially or simultaneously administered with at least one additional therapeutic agent. Examples of additional therapeutic agents include, but are not limited to, antiangiogenic agents, alkylating agents, platinum agents, taxanes, vinca agents, anti-estrogen drugs, aromatase inhibitors, ovarian suppression agents, VEGF/VEGFR inhibitors, EGF/EGFR inhibitors, PARP inhibitors, cytostatic alkaloids, cytotoxic antibiotics, antimetabolites, endocrine/hormonal agents, bisphosphonate therapy agents and targeted biological therapy agents (e.g., therapeutic peptides described in U.S. Pat. No. 6,306,832, WO 2012007137, WO 2005000889, WO 2010096603 etc.). In some embodiments, the at least one additional therapeutic agent is a chemotherapeutic agent. Specific chemotherapeutic agents include, but are not limited to, cyclophosphamide, fluorouracil (or 5-fluorouracil or 5-FU), methotrexate, edatrexate (10-ethyl-10-deaza-aminopterin), thiotepa, carboplatin, cisplatin, taxanes, paclitaxel, protein-bound paclitaxel, docetaxel, vinorelbine, tamoxifen, raloxifene, toremifene, fulvestrant, gemcitabine, irinotecan, ixabepilone, temozolmide, topotecan, vincristine, vinblastine, eribulin, mutamycin, capecitabine, anastrozole, exemestane, letrozole, leuprolide, abarelix, buserlin, goserelin, megestrol acetate, risedronate, pamidronate, ibandronate, alendronate, denosumab, zoledronate, trastuzumab, tykerb, anthracyclines (e.g., daunorubicin and doxorubicin), bevacizumab, oxaliplatin, melphalan, etoposide, mechlorethamine, bleomycin, microtubule poisons, annonaceous acetogenins, or combinations thereof.

Other examples of additional therapeutic agents include, but are not limited to, immune checkpoint inhibitors, monoclonal antibodies that specifically target tumor antigens, cell-mediated immunotherapy (e.g., T cell therapy), immune activating agents (e.g., interferons, interleukins, cytokines), oncolytic virus therapy and cancer vaccines. Examples of immune checkpoint inhibitors include immuno-modulating/stimulating antibodies such as an anti-PD-1 antibody, an anti-PD-L1 antibody, an anti-PD-L2 antibody, an anti-CTLA-4 antibody, an anti-TIM3 antibody, an anti-4-1BB antibody, an anti-CD73 antibody, an anti-GITR antibody, and an anti-LAG-3 antibody. Specific immuno-modulating/stimulating antibodies include ipilimumab, Nivolumab, Pembrolizumab, Atezolizumab, Avelumab, and Durvalumab. Additionally or alternatively, in some embodiments, the monoclonal antibodies that specifically target tumor antigens bind to one or more targets selected from among CD3, GPA33, HER2/neu, GD2, MUC16, MAGE-1, MAGE-3, BAGE, GAGE-1, GAGE-2, MUM-1, CDK4, N-acetylglucosaminyltransferase, p15, gp75, beta-catenin, ErbB2, cancer antigen 125 (CA-125), carcinoembryonic antigen (CEA), RAGE, MART (melanoma antigen), MUC-1, MUC-2, MUC-3, MUC-4, MUC-5ac, MUC-16, MUC-17, tyrosinase, Pmel 17 (gp100), GnT-V intron V sequence (N-acetylglucoaminyltransferase V intron V sequence), Prostate cancer psm, PRAME (melanoma antigen), β-catenin, EBNA (Epstein-Barr Virus nuclear antigen) 1-6, LMP2, p53, lung resistance protein (LRP), Bcl-2, prostate specific antigen (PSA), Ki-67, CEACAM6, colon-specific antigen-p (CSAp), HLA-DR, CD40, CD74, CD138, EGFR, EGP-1, EGP-2, VEGF, P1GF, insulin-like growth factor (ILGF), tenascin, platelet-derived growth factor, IL-6, CD20, CD19, PSMA, CD33, CD123, MET, DLL4, Ang-2, HER3, IGF-1R, CD30, TAG-72, SPEAP, CD45, L1-CAM, Lewis Y (Le^{y}) antigen, E-cadherin, V-cadherin, GPC3, EpCAM, DLL3, PD-1, PD-L1, CD28, CD137, CD99, GloboH, CD24, STEAPI, B7H3, Polysialic Acid, OX40, OX40-ligand, or other peptide MHC complexes (e.g., with peptides derived from TP53, KRAS, MYC, EBNAi-6, PRAME, MART, tyronsinase, MAGEA1-A6, pmel17, LMP2, or WT1). Examples of immune activating agents include, but are not limited to, interferon α, interferon β, interferon γ, complement C5a, IL-2, TNFalpha, CD40L, IL12, IL-23, IL15, IL17, CCL1, CCL11, CCL12, CCL13, CCL14-1, CCL14-2, CCL14-3, CCL15-1, CCL15-2, CCL16, CCL17, CCL18, CCL19, CCL19, CCL2, CCL20, CCL21, CCL22, CCL23-1, CCL23-2, CCL24, CCL25-1, CCL25-2, CCL26, CCL27, CCL28, CCL3, CCL3L1, CCL4, CCL4L1, CCL5, CCL6, CCL7, CCL8, CCL9, CCR10, CCR2, CCR5, CCR6, CCR7, CCR8, CCRL1, CCRL2, CX3CL1, CX3CR, CXCL1, CXCL10, CXCL11, CXCL12, CXCL13, CXCL14, CXCL15, CXCL16, CXCL2, CXCL3, CXCL4, CXCL5, CXCL6, CXCL7, CXCL8, CXCL9, CXCL9, CXCR1, CXCR2, CXCR4, CXCR5, CXCR6, CXCR7 and XCL2.

In any case, the multiple therapeutic agents may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may vary from more than zero weeks to less than four weeks. In addition, the combination methods, compositions and formulations are not to be limited to the use of only two agents.

### Kits

The present disclosure provides a kit comprising an expression vector comprising a nucleic acid encoding a P-selectin-specific chimeric receptor polypeptide comprising (a) an extracellular domain that specifically binds P-selectin; (b) a heterologous receptor polypeptide comprising one or more ligand-inducible proteolytic cleavage sites (e.g., Notch receptor polypeptide); and (c) an intracellular domain comprising a transcriptional activator (e.g., Gal4-VP16), wherein binding of the extracellular domain to P-selectin induces cleavage of the heterologous receptor polypeptide (e.g., Notch receptor polypeptide) at the one or more ligand-inducible proteolytic cleavage sites to release the intracellular domain. In some cases, a kit comprises a nucleic acid encoding any and all embodiments of the P-selectin-specific chimeric receptor polypeptide (e.g., a Notch receptor polypeptide) disclosed herein.

In some embodiments, the kits of the present technology further comprise a nucleic acid encoding a CAR polypeptide that is operably linked to a promoter that is responsive to the transcriptional activator of the released intracellular domain of the P-selectin-specific chimeric receptor polypeptide, or an expression vector comprising said nucleic acid. Additionally or alternatively, in some embodiments, the kits of the present technology further comprise instructions for transducing T cells with a nucleic acid encoding any and all embodiments of the P-selectin-specific chimeric receptor polypeptide (e.g., a Notch receptor polypeptide) disclosed herein (or an expression vector comprising said nucleic acid), and/or a nucleic acid encoding a CAR poly peptide that is operably linked to a promoter that is responsive to the transcriptional activator of the released intracellular domain of the P-selectin-specific chimeric receptor polypeptide (or an expression vector comprising said nucleic acid).

Kit components can be in the same container, or in separate containers. Any of the above-described kits can further include one or more additional reagents, where such additional reagents can be selected from: a dilution buffer; a reconstitution solution; a wash buffer; a control reagent; a control expression vector; a negative control polypeptide (e.g., a chimeric receptor polypeptide that lacks the one or more ligand-inducible proteolytic cleavage sites, such that, upon binding of the extracellular domain to IP-selectin, the intracellular domain is not released), a positive control polypeptide; a reagent for in vitro production of the chimeric receptor polypeptide, and the like.

The instructions for practicing the methods disclosed herein are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, flash drive, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### EXAMPLES

Those skilled in the art will recognize that several embodiments are possible within the scope and spirit of this present application. The present application will now be described in greater detail by reference to the following non-limiting examples. The following examples further illustrate the present application but, of course, should not be construed as in any way limiting its scope.

### Example 1: Experimental Materials and Methods

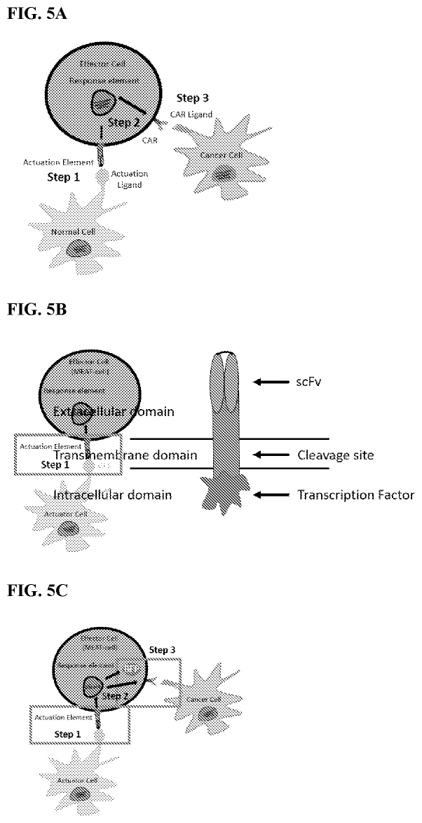

Approach: The MEAT-cell requires two target cells for it to become selectively cytotoxic: a normal “actuator” cell in the normal tissue, and a target cancer cell residing in that tissue. If these two conditions are met, the effector cell (MEAT-cell) will kill the target cancer cell (FIGS. 5A-5C). The MEAT-cells have genetically encoded actuation elements and response elements. The actuation element will express a receptor that binds to a cognate ligand present on actuator cells. The response element will encode a CAR molecule that is turned on following actuation. Therefore, IF a MEAT-cell engages with actuator cells in the metastatic tissue, it will express its CAR molecule, and THEN kill target cells; creating a “IF A” “THEN B” logic decision for the cell. In contrast, if the MEAT-cell is not actuated (not expressing the CAR molecule) and encounters the CAR antigen in a different environment, for example on a healthy, non-cancerous tissue cell, the MEAT cell will not kill.

MEAT-cell system design: MEAT cell systems were built by utilizing the two vector Gal4 SynNotch receptor technology. Through Gibson Assembly, we engineered the sensor element and response element into a single pHR lentiviral vector (MEAT). The SynNotch receptor utilized scFv sequences synthesized by Genscript. These include scFvs from Crizanlizumab (anti-human P-selectin) and from Biolegend (anti-mouse P-selectin antibody) Cat #148301. A GD2 reactive CAR vector was provided by the Milone group. Through Gibson Assembly, the GD2 reactive CAR was cloned into a constitutively expressing the pHR Lentiviral vector and into the response element of the MEAT System.

Primary Human T-cell isolation and culture: Human blood was collected from normal human donors. Lymphocytes were isolated using lymphocyte separation media and red blood cells were lysed with LCK lysis buffer. T-cells were cultured with IL-2 supplemented complete RPMI media.

Lentiviral Transduction of Human T-cells: Lenti-X 293T cells were plated in 10 cm poly-d-lysine coated TC treated plates at 6×10⁶ cells per plate. After 24 hr, Lenti-X cells were transfected with equimolar concentrations of pHR, PAX2, MD2.G plasmids in Optimem. After 6 hours of incubation, the media was replaced with 10 ml of complete RPMI. 24 hrs post transfection, media was once again replaced with 10 ml of complete RPMI. T-cells were stimulated for 48 hrs with anti-CD3 OKT3 antibody and IL-2 prior to transduction. The human T-cells were subsequently plated into 6-well non-treated TC plates coated with retronectin and viral supernatant was added with polybrene (final concentration of 4 ug/ml). Cells were spun at 2000 g for 1 hr at 30° C. and transferred to 37° C. incubator. Transduction was verified by flow-cytometric analysis using an anti-Myc antibody that binds to the Myc tag on the synNotch receptor.

Cancer Cell Lines: Jurkat, K562, and SK-N-BE(2) cells were ordered from ATCC.

Assessment of SynNotch AND-Gate T Cell Cytotoxicity: Transduction of the actuating element was confirmed with fluorescently labeled anti-Myc antibodies that bind to the Myc tag on the synNotch receptor.

### pHR CD19CAR (41BBz) Amino Acid Sequence

(SEQ ID NO: 100)
MALPVTALLLPLALLLHAEVKLQQSGAELVRPGSSVKISCKASGYAFSSY
WMNWVKQRPGQGLEWIGQIYPGDGDTNYNGKFKGQATLTADKSSSTAYMQLSGL
TSEDSAVYFCARKTISSVVDFYFDYWGQGTTVTVSSGGGGSGGGGSGGGGSDIELT
QSPKFMSTSVGDRVSVTCKASQNVGTNVAWYQQKPGQSPKPLIYSATYRNSGVPD
RFTGSGSGTDFTLTITNVQSKDLADYFCQQYNRYPYTSGGGTKLEIKRAAAPTTTPA
PRPPTPAPTIASQPLSLRPEACRPAAGGAVHTRGLDFACDIYIWAPLAGTCGVLLLSL
VITLYCNKRGRKKLLYIFKQPFMRPVQTTQEEDGCSCRFPEEEEGGCELRVKFSRSA
EPPAYQQGQNQLYNELNLGRREEYDVLDKRRGRDPEMGGKPRRKNPQEGLYNEL
QKDKMAEAYSEIGMKGERRRGKGHDGLYQGLSTATKDTYDALHMQALPPRS

### Example 2: GD2-Reactive CAR T-Cells Cause Neurological Toxicities Through On-Target, Off Tumor Killing

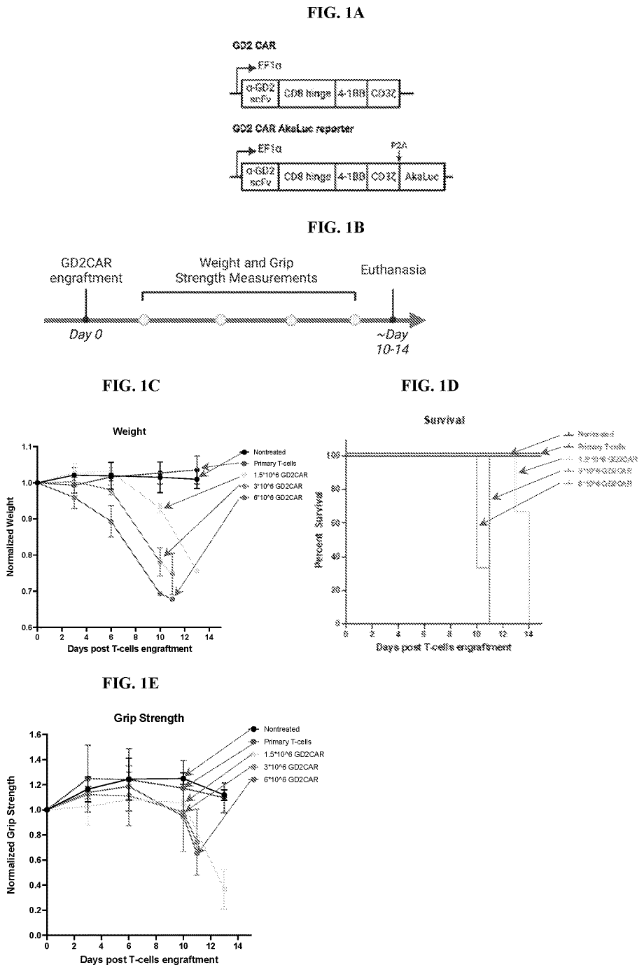

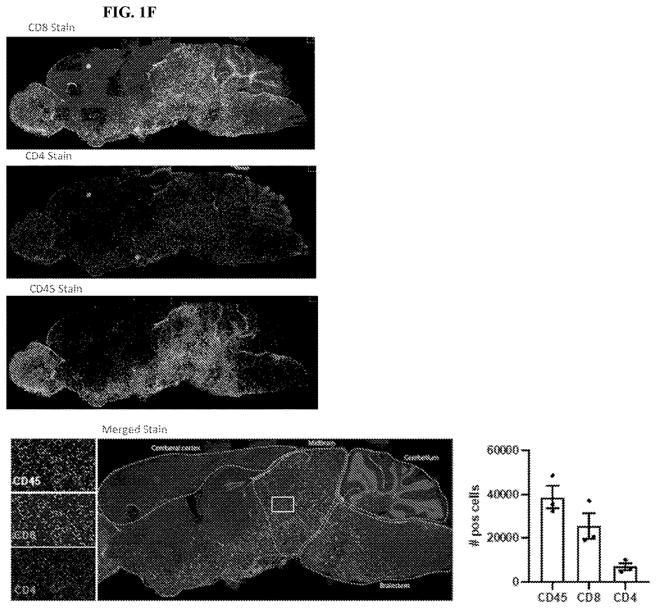

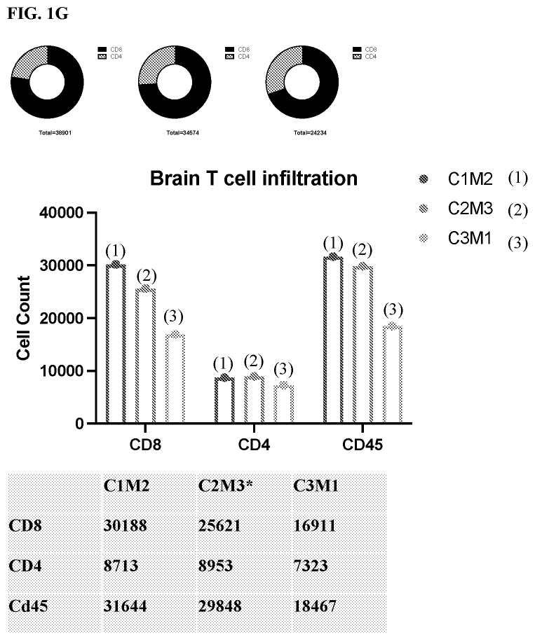

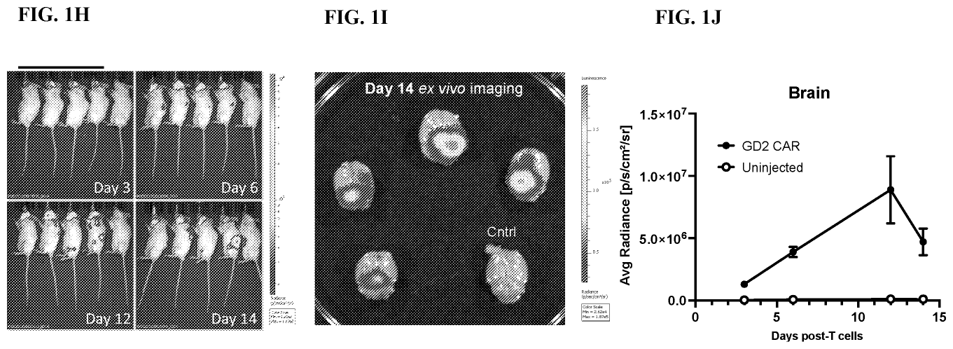

Previous work has reported fatal encephalitis toxicities through treatment of NSG mice with a high-affinity E101K mutated scFv GD2-reactive CAR T-cells (GD2 CAR). Richman, S. A. et al., Cancer Immunology Research, (2018) 6 (1): 36-46. GD2 CAR was cloned into a pHR lentiviral expression vector and which was used to transduce primary human T cells from healthy donors (FIG. 1A). To determine if the GD2 CAR causes toxicity as previously reported, we engrafted retro-orbitally human primary T-cells transduced with GD2 CAR (FIG. 1B). The mice weights were measured periodically and were euthanized upon 20% weight loss or through succumbing to treatment (FIG. 1C). All mice treated with GD2 CAR at all dosage levels tested died, though, at surprisingly earlier timepoints of ~2 weeks (FIG. 1D) versus the previously published results of ~3 weeks.

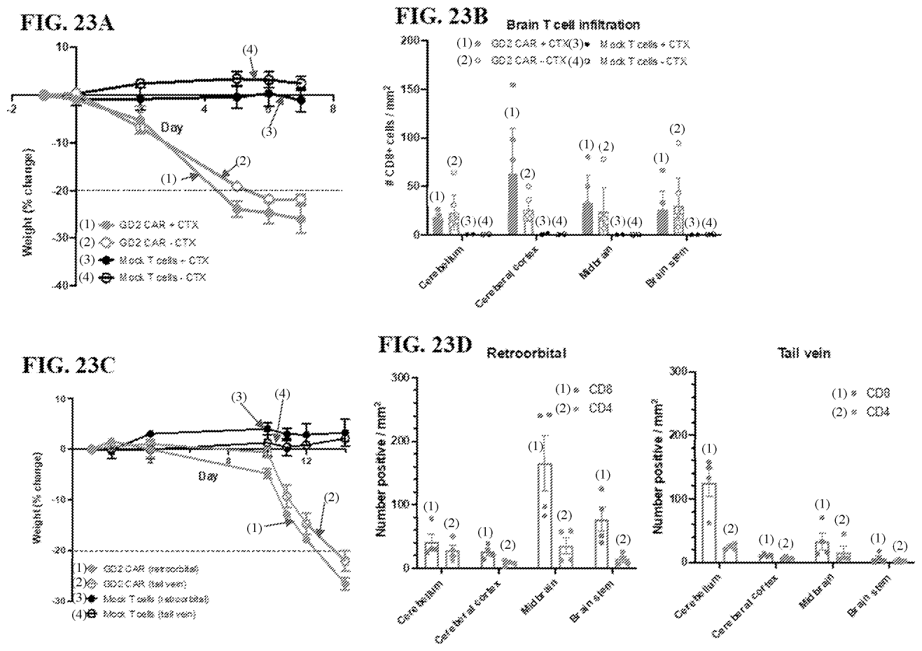

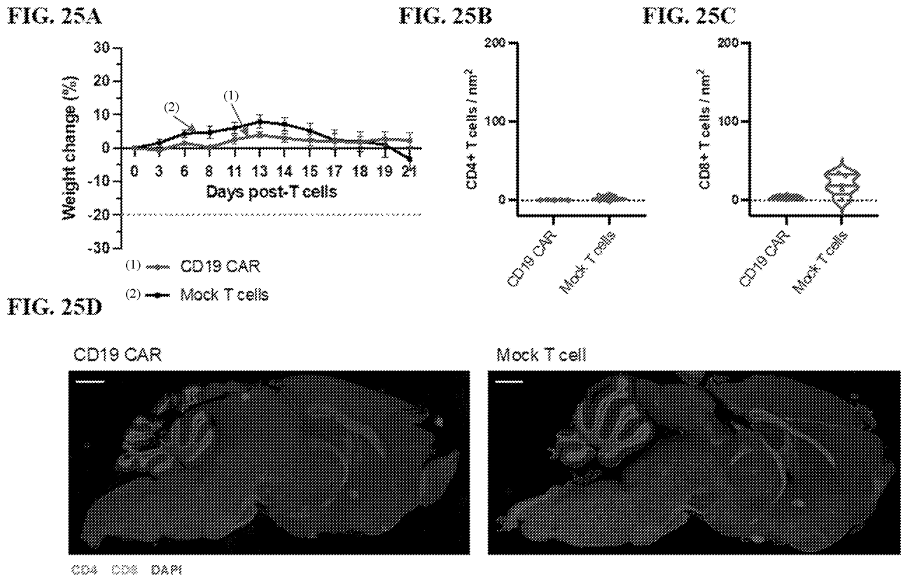

At the endpoint, the brains were harvested and stained with immunofluorescent histochemistry. Of note, GD2 CAR treated mice began to display motor function abnormalities such as hind limb paralysis (FIG. 1E) correlating to their steep drop off in weight and subsequent death, consistent with signs of neurological toxicities. Control mice injected with CD19 CAR T cells did not demonstrate weight loss or significant T cell brain infiltration (FIGS. 25A-25D). To investigate, 3×10⁶ GD2 CAR T-cells were engrafted i.v. and mice were sacrificed at Day 1, 5, 9, 12, 14. Mouse spleens were collected to examine T-cell expansion and their brains were collected to examine for T-cell infiltration through immunofluorescent histological analysis. Like previously published results, we observed infiltration of CD45+ T-cells into the cerebellum and midbrain with limited infiltration into the cerebral cortex (FIG. 1F). Upon further investigation CD8+ T-cells were the predominant CD45+ population with an approximate 3:1 ratio compared to CD4+ T-cells (FIG. 1G). It was further found that there was no difference in brain infiltration with cyclophosphamide (CTX) preconditioning of the mice or when injecting CAR T cells via different intravenous routes (FIGS. 23A-23D). These data are consistent with previous reports and show that engraftment of constitutively expressed GD2-reactive CAR T-cells leads to fatal encephalitis in NSG mouse models.

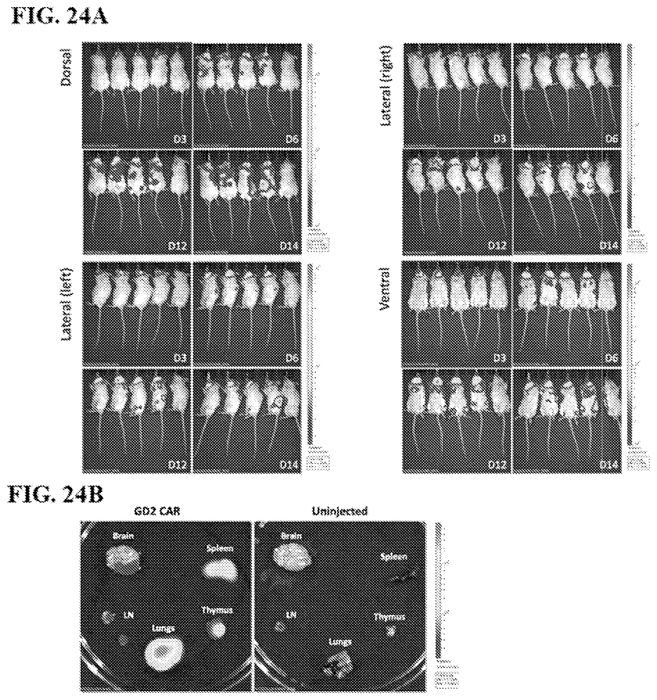

Mice were then injected with GD2 CAR T cells expressing AkaLuc as a reporter and tracked via bioluminescence imaging (BLI) to determine kinetics and biodistribution of CAR expansion. GD2 CAR T cells localized to the brain by day 3 and continued to expand through the endpoint at day 14 (FIGS. 1H-1J). Brain-specific localization was confirmed by ex vivo imaging of the whole brain after injection of the AkaLuc substrate AkaLumine-HCl. CAR T cells appeared to also expand systemically, as shown by signal in the spleen on day 6 and lymph nodes, thymus, and lungs on day 12 (FIGS. 24A-24B).

### Example 3: P-selectin Induces GD2 CAR Production in MEAT^{(αm/mSELP_GD 2CAR)})

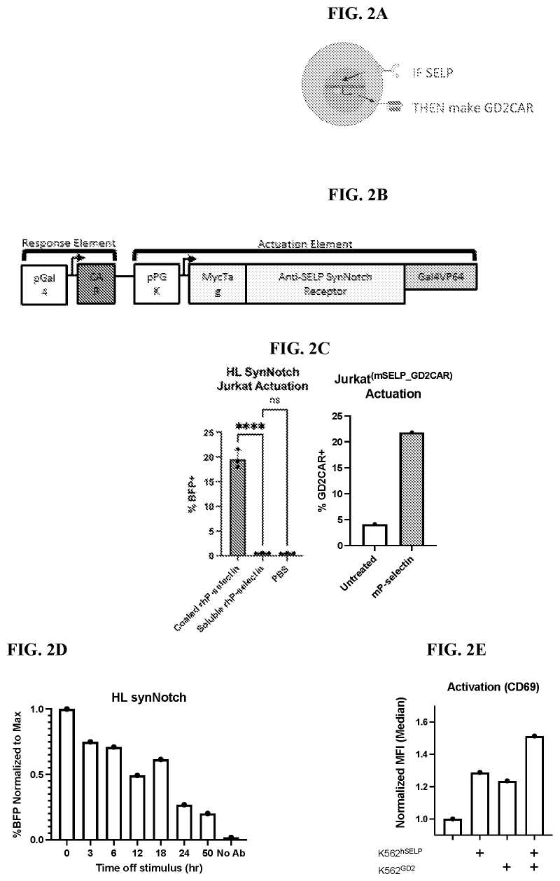

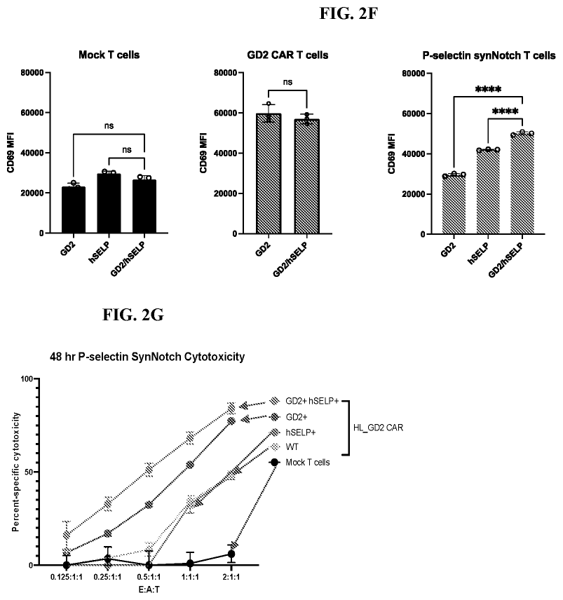

The MEAT-cell system is designed to bind to two auxiliary cells for it to become selectively cytotoxic: a normal “actuator” cell in the tumor microenvironment, and a target cancer cell residing in that tumor microenvironment. If these two conditions are met, the effector cell (MEAT-cell) will kill the target cancer cell (FIG. 2A). The MEAT-cells genetically encode actuation elements and response elements. The actuation element expresses a receptor that binds to a cognate ligand human P-selectin (hSELP) or mouse P-selectin (mSELP). The response element encodes a GD2-reactive CAR molecule that is turned on following actuation (MEAT^{(αh/mSELP_GD2 CAR)}) (FIG. 2B). To test the MEAT systems, Jurkat cells were transduced with MEAT systems containing P-selectin sensors for human and mouse variants.

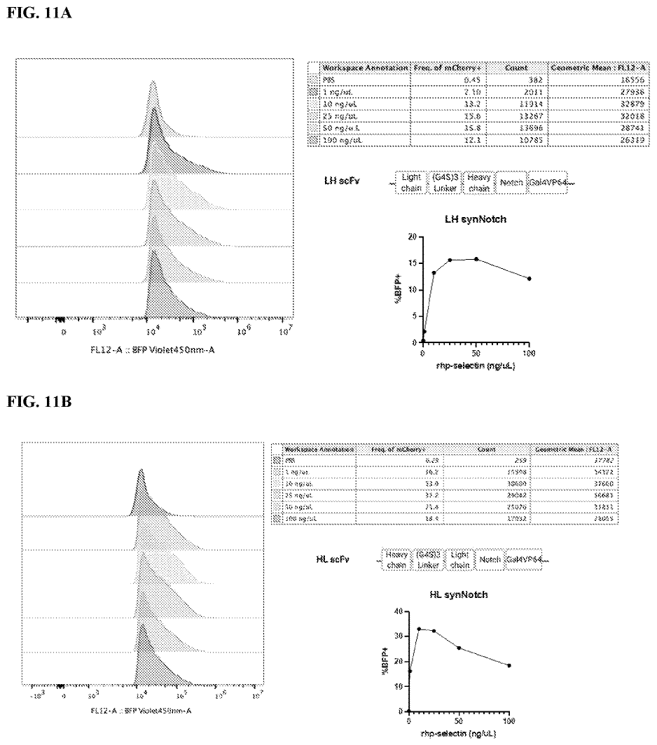

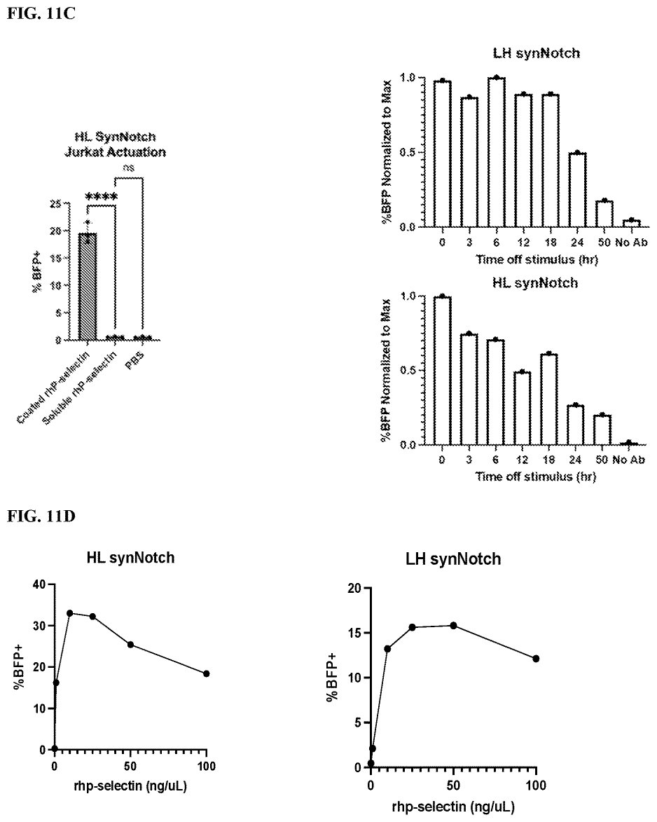

We tested the actuability of the HL Crizanlizumab Combo and LH Crizanlizumab Combo constructs by transducing Jurkat cell lines and culturing them on a hSELP coated non-tissue culture treated 96 well plate. Both constructs yielded MEAT systems capable of actuation as measured through induction of BFP (FIGS. 11C-11D). Co-culture with soluble form of hSELP did not induce actuation, demonstrating HL synNotch ligands must be tethered to induce actuation (FIG. 11C).

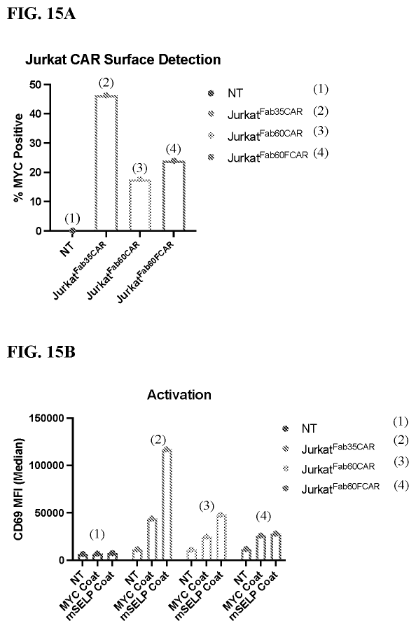

We obtained a monoclonal anti-mSELP antibody from Biolegend and obtained its amino acid sequence using mass-spectrometry. We designed multiple variations of anti-P-selectin Fabs, incorporated them into CAR constructs, and transduced Jurkat cells. Expression levels were tested using an anti-MYC antibody. Out of the four variations, three variations (Jurkat^{Fab35}, Jurkat^{Fab60}, Jurkat^{Fab60F}) showed surface expression of our CAR (FIG. 15A). Next, we tested the ability of Jurkat^{Fab35}, Jurkat^{Fab60}, Jurkat^{Fab60F} cells to bind to recombinant mSELP using our mSELP coated plate assay. All three variants activated in response to mSELP (FIG. 15B).

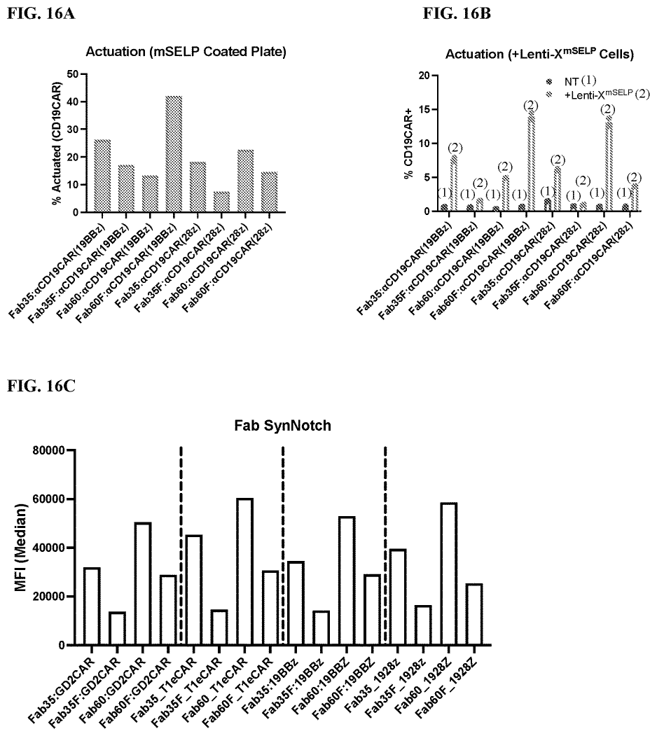

Next we wanted to see if incorporating our anti-mSELP Fab sequences into synNotch receptors would result in functional actuation in the presence of mSELP. We designed constructs with each variation of our Fab sequences into MEAT Barebones constructs containing inducible αGD2CAR, T1eCAR, αCD19CAR (19BBz) and αCD19CAR (1928z). All constructs were detectable upon transduction into Jurkat cells through flow cytometric analysis using an anti-murine Fab antibody (FIG. 16C). We took the MEAT^{anti-mSELP Fab:αCD19CAR (19BBz)/αCD19CAR (1928z)} Jurkat cell variants and cultured them on a mSELP coated plate and on Lenti-X^{mSELP} cells. All forms actuated and produced their respective αCD19CARs (FIGS. 16A-16B). All Jurkats transduced with our anti-mSELP Fab MEAT systems transduced with different efficiencies, thus making it difficult to compare between all variations and chose our best synNotch receptor to move forward. Due to the highest detection signal by flow cytometry (strongest MFI signal in positively transduced cells), we chose the Fab60 based constructs for our future experiments (FIG. 16C).

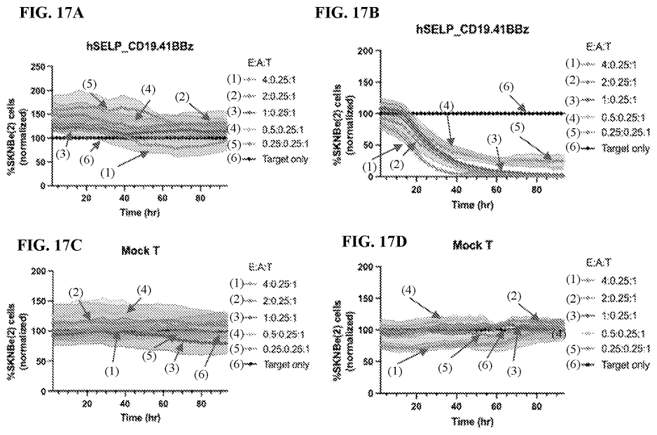

Having developed MEAT-cell^{HL:αCD19CAR} and MEAT-cell^{Fab60:αCD19CAR} systems capable of inducing αCD19CAR expression upon the presence of hSELP and mSELP, respectively. Next we aimed to show that we can selectively kill CD19+ tumors in vitro. First, we co-cultured primary human T-cells^{HL:αCD19CAR} at increasing effector ratios with combinations of actuator Lenti-X^{hSELP} and Lenti-X^{mSELP} (control) cells and target cells SK-n-Be(2)^{CD19} and SK-n-Be(2)^{WT}. Only when primary human T-cells^{HL:αCD19CAR} where cocultured with Lenti-X^{hSELP} cells did they kill their target cells. In all other conditions, the primary human T-cells^{HL:αCD19CAR} did not kill the SK-n-Be(2)^{CD19} or the SK-n-Be(2)^{WT}. These results indicate that primary human T-cells^{HL:αCD19CAR} cells selectively kill antigen positive cells only in the presence of their actuator, hSELP (FIGS. 17A-17D).

Upon incubation with tissue culture plates coated with their respective recombinant proteins, the MEAT Jurkat cells produced GD2-reactive CAR (FIG. 2C). The soluble form of these proteins did not induce expression of GD2-reactive CARs on MEAT Jurkats cells, demonstrating synNotch ligands must be tethered to induce actuation. Upon removal of the MEAT Jurkats from the SELP coated plates, GD2-reactive surface expression decreased with a half-life of ~12 hrs (FIG. 2D).

Next, we tested if our MEAT Jurkats could sense SELP on a cell, make GD2-reactive CAR, and activate upon binding to another cell expressing GD2. We incubated MEAT Jurkats with combinations of wildtype K562 cells, K562 cells expressing hSELP (K562^{SELP}) or mSELP (K562^{mSELP}), and K562 cells expressing GD2 (K562^{GD2}). Induction of GD2-reactive CAR expression was only detected in the presence of K562^{hSELP} and a substantial increase in activation was only detected when Jurkat MEAT cells were co-cultured with K562^{hSELP} and K562^{GD2} (FIG. 2E). Therefore, the MEAT-cell system disclosed herein can engage with P-selectin expressing actuator cells, produce the GD2 CAR molecule and activate upon binding to GD2 expressing target cells.

To show that the MEAT-cell system can contextually kill target cancer cells, primary T-cells were transduced with the MEAT-cell system (primary MEAT-cells). When co-cultured with dual antigen K562^{hsELP_GD2} cells, primary MEAT-cells expressed GD2-reactive CAR and activated, demonstrating the MEAT system is functional in primary human cells (FIG. 2F). To test selective killing of target cells, primary MEAT-cells were incubated with combinations of wildtype K562 cells, K562^{hSELP} cells, and K562^{GD2} cells. Target K562^{GD2} cells were only killed by primary MEAT-cells in the presence of actuator K562^{hSELP} cells (FIG. 2G). Therefore, the MEAT-cell system described herein can engage with actuator cells, express its CAR molecule and kill antigen expressing cells. These data demonstrate that the MEAT^{(hSELP_GD2 CAR)} system is functional and enables selective killing by human primary MEAT-cells.

### Example 4: Primary Human MEAT^{(hSELP_GD2 CAR)} Cells Ameliorate GD2 CAR Toxicity

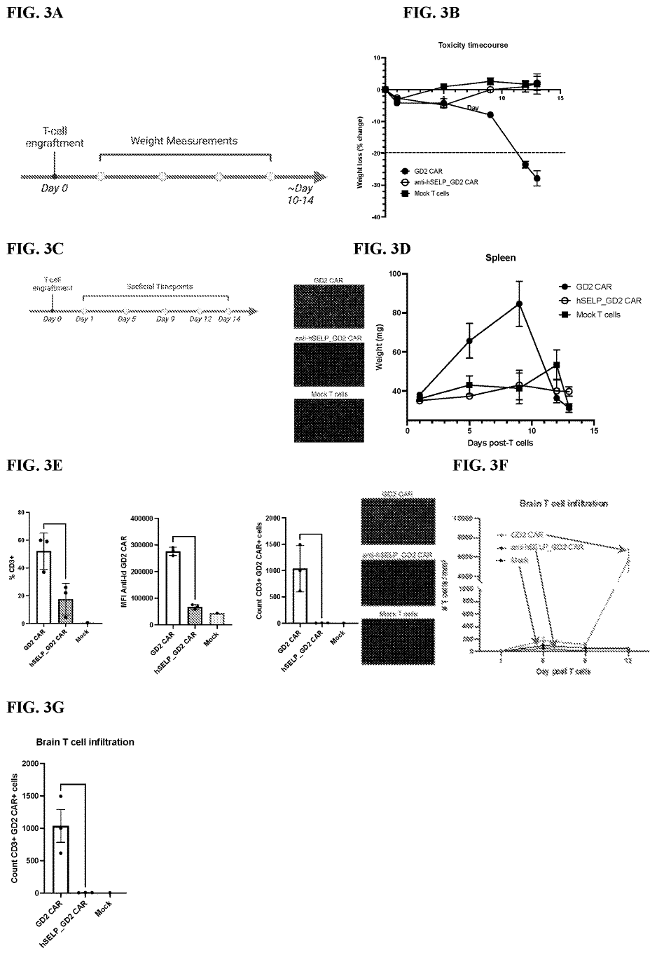

Since the MEAT^{(hSELP_GD2 CAR)} system is capable of restricting GD2 CAR expression in vitro, we next aimed to show this control is maintained in vivo. Human primary MEAT-cells were transduced with the MEAT^{(hSELP_GD2 CAR)} system and engrafted them into NSG mice (FIG. 3A). The mice's weight were measured periodically and were euthanized upon 20% weight loss or through succumbing to treatment. As consistent with previous results, within 2 wks mice engrafted with control T-cells transduced with GD2-reactive CARs displayed lethal toxicity (FIG. 3B). However, the mice engrafted with T-cells transduced with the MEAT^{(hSELP_GD2 CAR)} system showed no weight loss or clear signs of neurotoxicity (FIG. 3B).

To investigate further, human primary MEAT-cells were transduced with the MEAT^{(hSELP_GD2 CAR)} system and engrafted them into NSG mice. Mice were sacrificed at predetermined time points (FIG. 3C) and tissues were collected for histological staining and flow cytometric analysis (FIGS. 3D-3F). In the control T-cells transduced with GD2-reactive CARs, we observed a larger increase in splenic weight overtime followed by a sharp decline right before the mice succumbed to treatment. The mice treated with T-cells transduced with the MEAT^{(hSELP_GD2 CAR)} system had no difference in splenic weight compared to non-transduced T-cell engrafted mice (FIG. 3D). The T-cell infiltrate in the spleen of T-cells transduced with the MEAT^{(hSELP_GD2 CAR)} system showed no substantial expression of GD2 CAR, indicating these T-cells are not actuated (FIG. 3E). The on-target, off-tumor brain tissue showed little T-cell infiltration of T-cells transduced with the MEAT^{(hSELP_GD2 CAR)} and no substantial expression of GD2 CAR was detected (FIGS. 3F-3G).

These results demonstrate that the MEAT-cell systems of the present technology showed contextual expression of GD2-reactive CARs in tumor microenvironment, eliminate tumor cells expressing CAR ligand, and mitigate on-target, off-tumor toxicity.

### Example 5: MEAT-Cell Line is Capable of Selective Actuation and Activation In-Vitro

GFP is not expressed on the surface of normal mammalian cells and therefore an anti-GFP synNotch receptor was used as a unique actuating element in our proof-of-concept model. The CD19 reactive CAR is a well-established CAR and thus was a suitable platform for the response element in the proof-of-concept model. Using lentiviral transduction, we created an anti-GFP:CD19CAR MEAT-cell Jurkat T-cell line that binds to extracellular GFP by an anti-GFP synNotch receptor and expresses a CD19 reactive CAR (Jurkat^{αGFP:CD19CAR}).

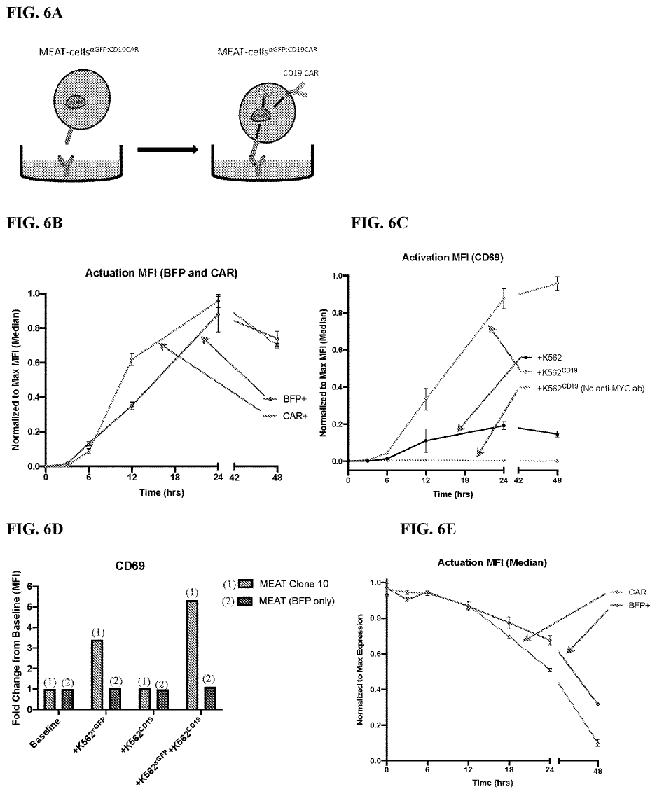

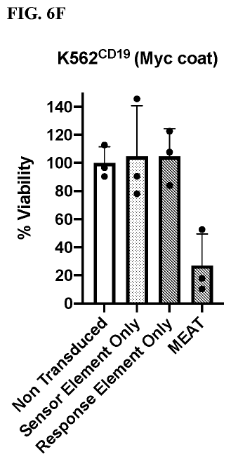

The ability of the Jurkat^{αGFP:CD19CAR} cells to actuate was tested by culturing them on plates coated with anti-MYC antibodies—where interaction of the cells with the anti-Myc antibody replicated engagement of the synNotch receptor (FIG. 6A). Actuation was measured using either a TagBFP reporter gene and/or with a fluorescently labeled anti-idiotype antibody to the anti-CD19 CAR. When Jurkat^{αGFP:CD19CAR} cells are cultured on plates coated with anti-MYC antibodies, we observed associated TagBFP and CD19CAR expression (FIG. 6B). When Jurkat^{αGFP:CD19CAR} cells were actuated on anti-MYC antibody coated plate for 24 hrs and then subsequently removed, we also observed associated decay kinetics between the CD19 CAR and TagBFP (FIG. 6E). Therefore, either measuring CD19CAR with an anti-idiotype ab directly or through TagBFP fluorescence allows for us to determine the actuation state of MEAT cells. When Jurkat^{αGFP:CD19CAR} cells actuated on anti-MYC antibody coated plates were co-cultured with K562 cells, we observed substantial CD69 activation only in the presence of K562 cells expressing truncated CD19 (K562^{CD19}) (FIG. 6C).

Next we tested the MEAT-cells' ability to actuate and activate in the presence of actuator and target cells. Jurkat^{αGFP:CD19CAR} cells are not actuated or activated by K562^{CD19} cells. When Jurkat^{αGFP:CD19CAR} cells were cocultured with K562GFP actuator cells, TagBFP expression ensued. In the presence of actuator K562^{GFP} and target^{K562CD19} cells, we see substantial increases in activation of Jurkat^{αGFP:CD19CAR} cells: suggesting that the αGFP:CD19CAR MEAT-cell gating mechanism is functional (FIG. 6D). Jurkat^{αGFP:CD19CAR} cells can engage with anti-myc coated plates or with actuator cells expressing a membrane-bound form of GFP, subsequently express CD19 reactive CARs, and then express T-cell activation markers upon binding to CD19 positive target cells in-vitro.

### Example 6: Ligands from Actuator Cells can Induce Expression of CARs in Effector Cells (MEAT-Cells) which Will Subsequently Bind to and Kill Target Cancer Cells

Human primary T-cells were transduced with the αGFP:CD19CAR MEAT system (T-cells^{αGFP:CD19CAR}). Using our proof of concept human primary T-cell^{αGFP:CD19CAR} system, T-cell CAR expression can be actuated by an anti-MYC coated plate (FIG. 6A). When human primary T-cells transduced with only the response element or actuator element are cultured target cells (K562^{CD19}) no killing is observed compared to non-transduced T-cells. When primary T-cells^{αGFP:CD19CAR} are actuated with an anti-MYC coated plate and co-cultured with K562^{CD19} target cells, killing is observed (FIG. 6F).

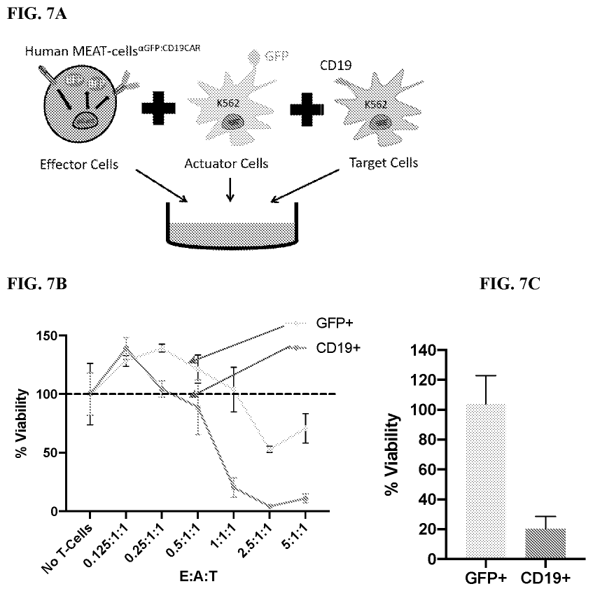

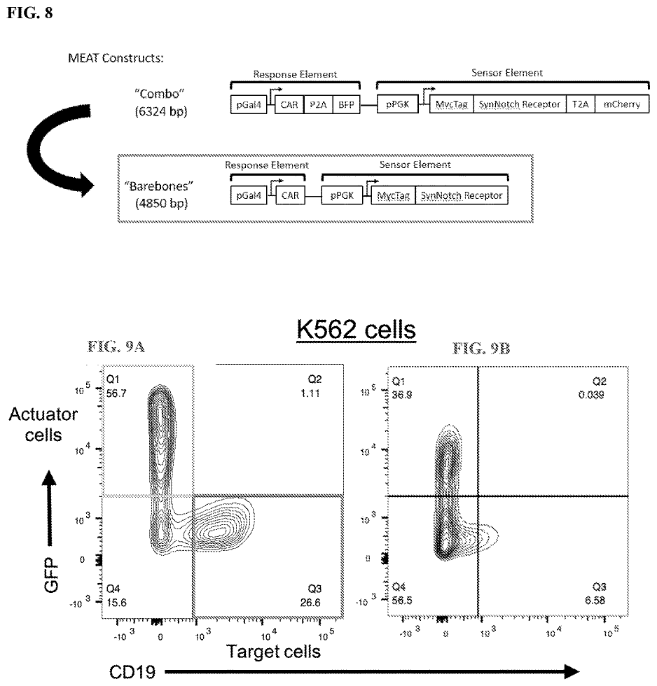

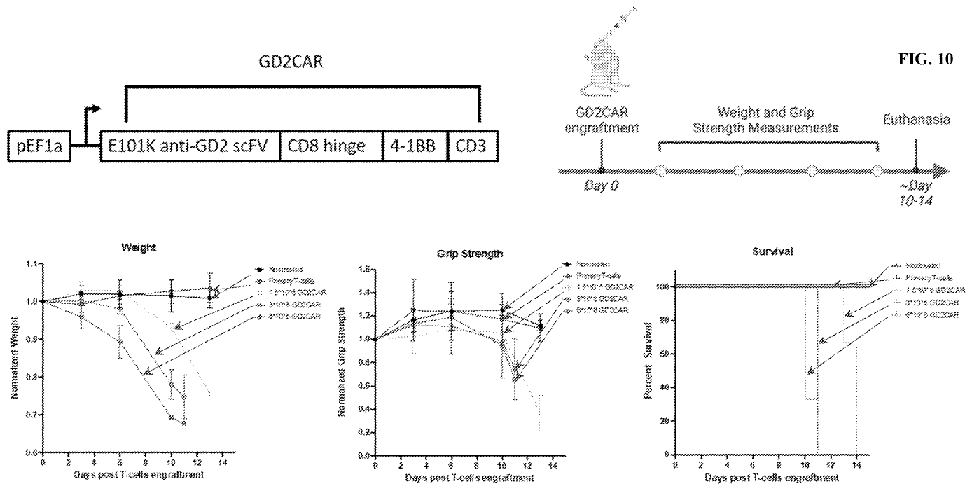

When human primary T-cells with only the response element or actuator element are cultured with actuator (K562^{GFP}) and target cells (K562^{CD19}), no actuation and preferential killing is observed (data not shown). When primary T-cells^{αGFP:CD19CAR} are cultured with K562 (no actuation ligand) and K562^{CD19} target cells, no actuation or preferential killing is observed (data not shown). However, in the presence of actuator (K562^{GFP}) and target (K562^{CD19}) cells (FIG. 7A), we see preferential killing of target cells by primary T-cells^{αGFP:CD19CAR} (FIGS. 7B-7C). These data suggest that the αGFP:CD19CAR MEAT-cell system is capable of properly gating CAR expression to kill target cells in the presence of actuator cells. Primary T-cells^{αGFP:CD19CAR} can be actuated by cells expressing a membrane-bound form of GFP, subsequently express CD19 reactive CARs, and kill CD19 positive target cells in-vitro.

### Example 7: P-Selectin is a Novel Antigenic Actuator in the Tumor Microenvironment and Construction of an αP-Selectin synNotch

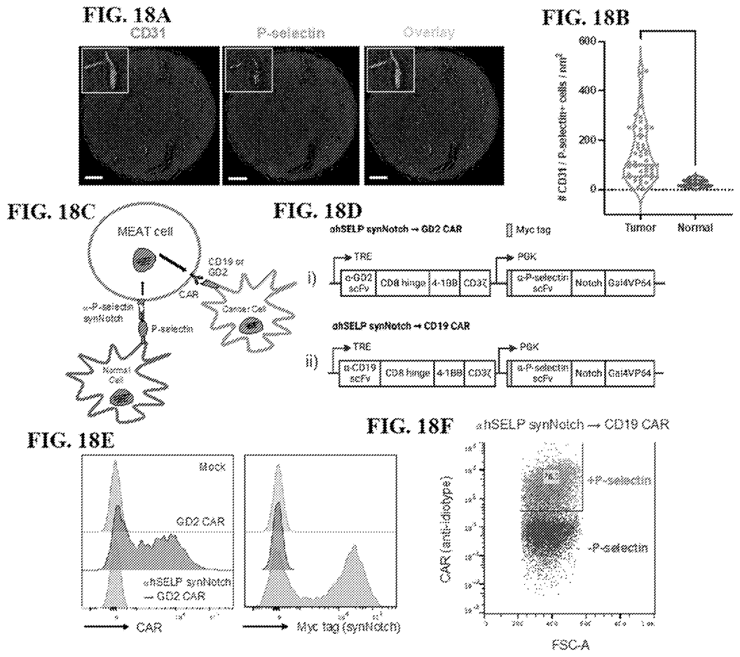

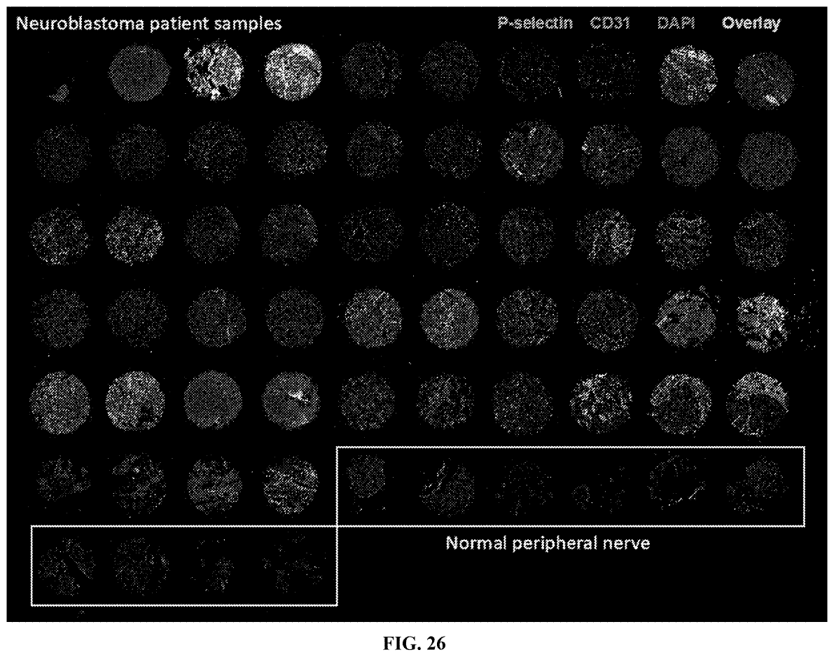

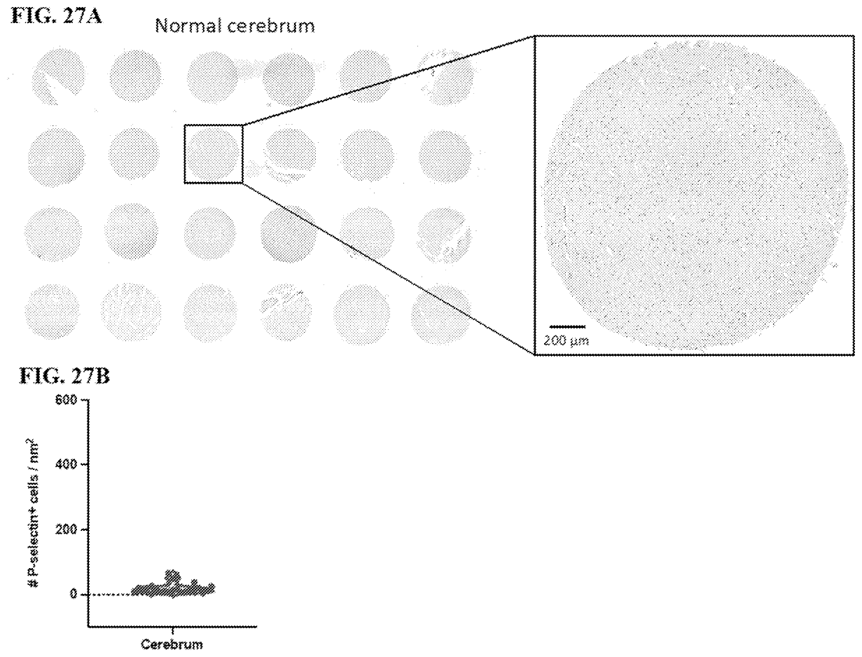

We next sought to identify an antigenic target in the TME that is highly expressed in a wide range of cancer types to use as a synNotch actuator. We investigated the cell adhesion molecule P-selectin due to its high expression in solid tumor stroma and biological role in T cell homing to sites of inflammation. Histological staining of P-selectin in 27 primary neuroblastoma patient samples showed high expression of P-selectin on CD3⁺ endothelial cells (FIG. 18A). In a subset of patients, P-selectin was also observed at high levels on tumor or other stromal cells (FIG. 26). Importantly, there was minimal quantitative expression of P-selectin on normal peripheral nerve and brain tissue relative to tumor, minimizing potential off-tumor actuation and toxicity in normal GD2⁺ tissue (FIG. 18B and FIG. 27A-27B).

We next engineered MEAT cells to express an anti-human P-selectin (αhSELP) synNotch to control CAR expression. We hypothesize that MEAT cells expressing an αhSELP synNotch actuate in the presence of human P-selectin to transcribe, translate, and express the CAR on the surface and selectively kill antigen positive cancer cells in the local environment (FIG. 18C). The scFv for Crizanlizumab, an FDA-approved αP-selectin antibody used to treat patients with sickle cell anemia, was used to generate a P-selectin-specific synNotch. G. P. Linette et al., Cardiovascular toxicity and titin cross-reactivity of affinity-enhanced T cells in myeloma and melanoma. Blood 122, 863-871 (2013).

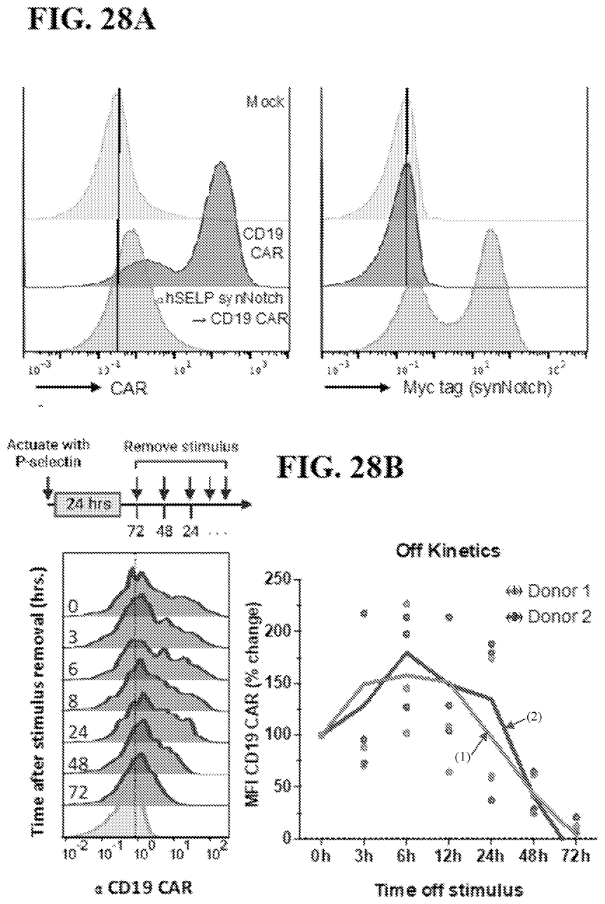

We cloned the αhSELP synNotch into a single two-part vector with 1) a constitutive response element containing the αP-selectin synNotch and 2) an actuation element beneath the conditional TRE promoter with either the CD19 or GD2 CAR (FIG. 18D). Primary human T cells were transduced with P-selectin sensing synNotch constructs and synNotch and CAR surface expression was validated by flow cytometry (FIG. 18E and FIGS. 28A-28B). We then cultured Jurkat MEAT cells with plate-bound human P-selectin and assessed actuation after 24 hours. P-selectin-specific CD19 CAR expression was observed with >75% of synNotch⁺ cells actuating and presenting CAR on the surface (FIG. 18F).

### Example 8: αP-Selectin synNotch Cells are Specific, Sensitive, and Cytotoxic In Vitro

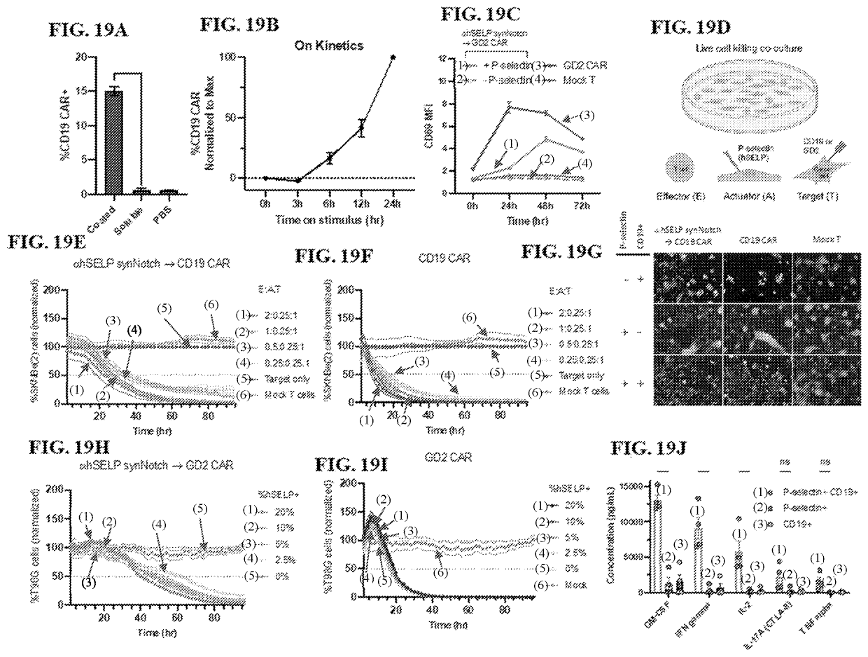

We next sought to characterize MEAT cell actuation, activation and killing kinetics. We found that primary MEAT cells only expressed CD19 CAR in the presence of plate-bound P-selectin (FIG. 19A). Importantly, soluble P-selectin did not induce CAR expression, likely due to a lack of mechanical force necessary to expose protease cleavage sites. This observation is critical to eliminate off-tumor CAR expression in physiological environments where soluble P-selectin might circulate in the plasma to promote inflammation and coagulation. CAR surface expression increased over 24 hours post-stimulation with target cells (FIG. 19B) and decreased quickly over time in the absence of target, with no detectible CAR expression at 72 hours, suggesting minimal off-tumor killing should T cells migrate to the brain after engaging with P-selectin in the TME (FIGS. 28A-28B).

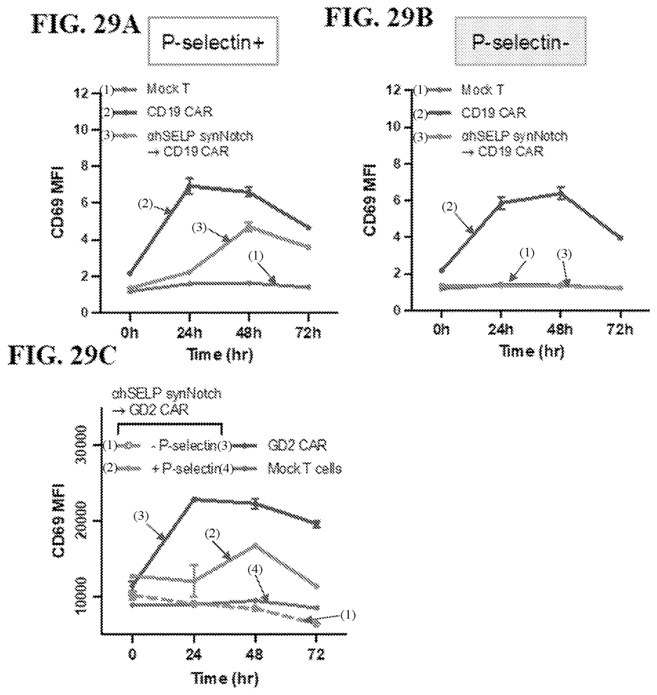

We then established a T cell co-culture with GD2+ T98G glioblastoma target cells to monitor CAR T cell activation kinetics. Constitutive GD2 CAR T cells activated rapidly within the first 24 hours, then decreased in activation over the following 48 hours, as measured by CD69 surface expression (FIG. 19C and FIGS. 29A-29C). Conversely, αhSELP synNotch→GD2 CAR T cells showed delayed activation with a peak at 48 hours. Importantly, there was no CD69 expression detected on synNotch CAR T cells in the absence of P-selectin, demonstrating actuator-specific activation (FIG. 19C dashed line).

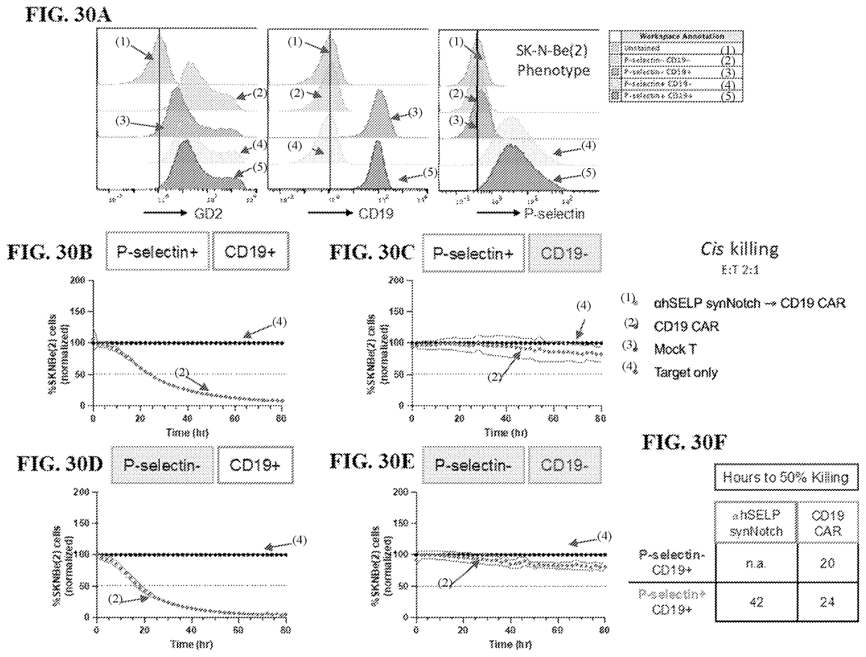

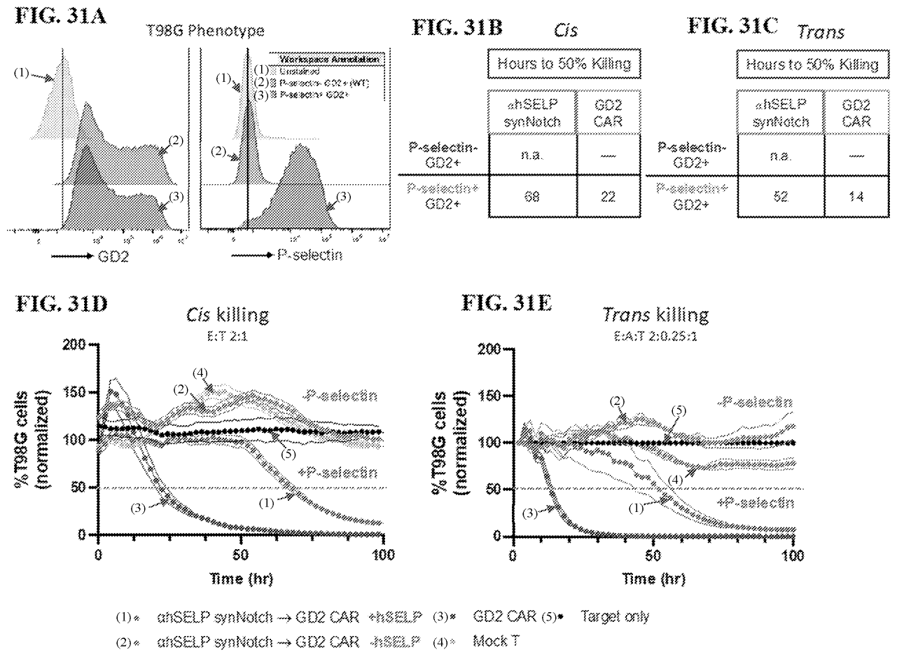

Next, we established a live-cell co-culture assay with effector cells, LentiX actuator cells (stably expressing P-selectin) and SK-N-BE(2) neuroblastoma target cells (CD19⁺ or GD2⁺) to understand kinetics of target cell killing (FIG. 19D). Specific and dose-dependent killing was observed for both αhSELP synNotch→CD19 CAR and constitutive CD19 CAR T cells at varying effector to actuator to target cell ratios (E:A:T) (FIGS. 19E-19F). An approximate 15-24 hr delay in 50% killing was observed for synNotch CAR T cells due to the requirement for de novo biosynthesis of the CAR after engaging with P-selectin, as compared to the constitutive CAR T cells in which the CAR is already present on the cell surface. These data support the activation kinetics previously described, which also show a 24-hour delay in activation. Importantly, αhSELP synNotch→GD2 CAR T cells only killed in the presence of both actuator and target ligands, as shown by killing of SK-N-BE(2) (CD19⁺ mCherry⁺) target cells after a 72 hour co-culture, and spared antigen negative GFP⁺ “bystander” cells (FIG. 19G). Live-cell co-culture experiments were repeated in both cis (P-selectin and CAR target on the same cell) and trans (P-selectin and CAR target on different cells) using PBMCs from additional healthy donors and similar trends were observed (FIGS. 30A-30F and 31A-31E).

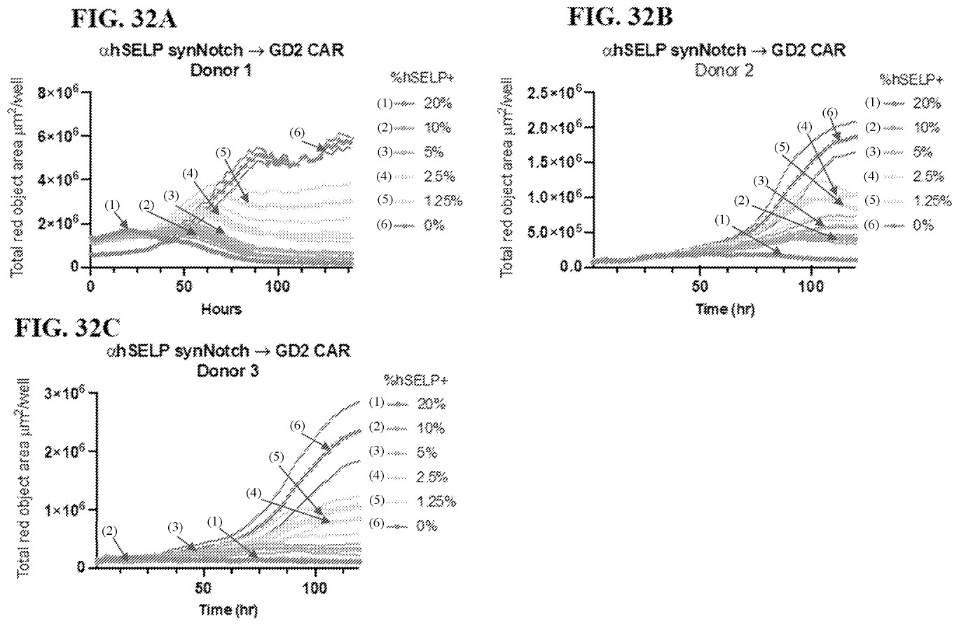

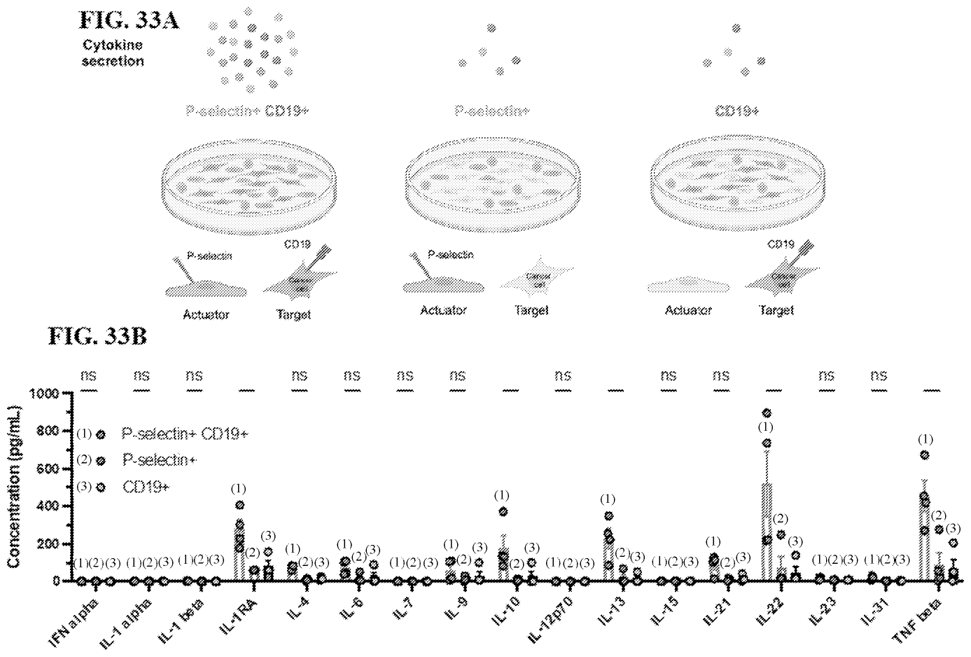

We then titrated the percentage of P-selectin positive actuator cells to probe the sensitivity of our synNotch to activate in response to decreasing abundance of actuator ligand. Though the time to 50% killing was delayed with decreasing frequency of actuator positive cells, αhSELP synNotch→GD2 CAR T cells were able to completely eradicate target cells with as low as 2.5% P-selectin⁺ actuator cells present (FIG. 19H and FIGS. 32A-32C). Conversely, constitutively active GD2 CAR T cells showed no dependence on decreasing concentration of P-selectin present on target cells (FIG. 19I). Furthermore, we observed specific cytokine secretion dependent on the presence of both actuator (P-selectin) and target (CD19) ligands, as measured by Luminex using a 25-plex human cytokine array. The five highest secreted cytokines included GM-CSF, IFNγ, IL-2, CTLA-8 and TNFα, all inflammatory cytokines associated with T cell activation and cytotoxic function (FIG. 19J). Incubation of synNotch T cells with target cells expressing either actuator or target ligand failed to produce an inflammatory response, supporting the lack of target cell killing in the presence of only one ligand. Similar trends were observed in additional cytokines profiled, including IL22, TNFβ, IL13, IL10 and IL1RA (FIGS. 33A-33B). These data demonstrate that αhSELP synNotch T cells maintain cytotoxicity of the gated CAR specifically in the presence of P-selectin and that only a small fraction of the tumor would need to be P-selectin⁺ for the cells to activate and kill the cancer cells.

### Example 9: Conditional CAR Expression Improves T Cell Fitness and Functionality Over Constitutive CAR Expression

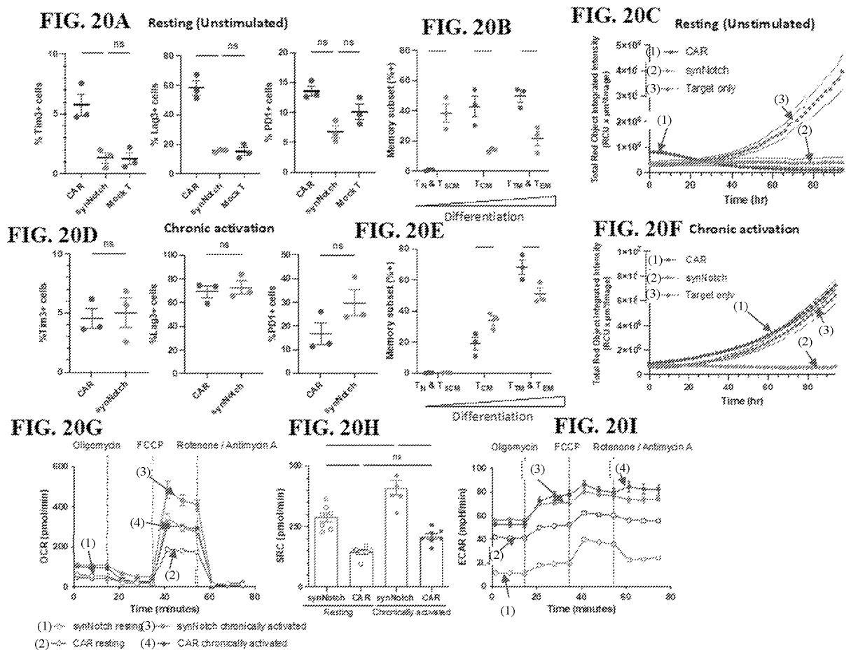

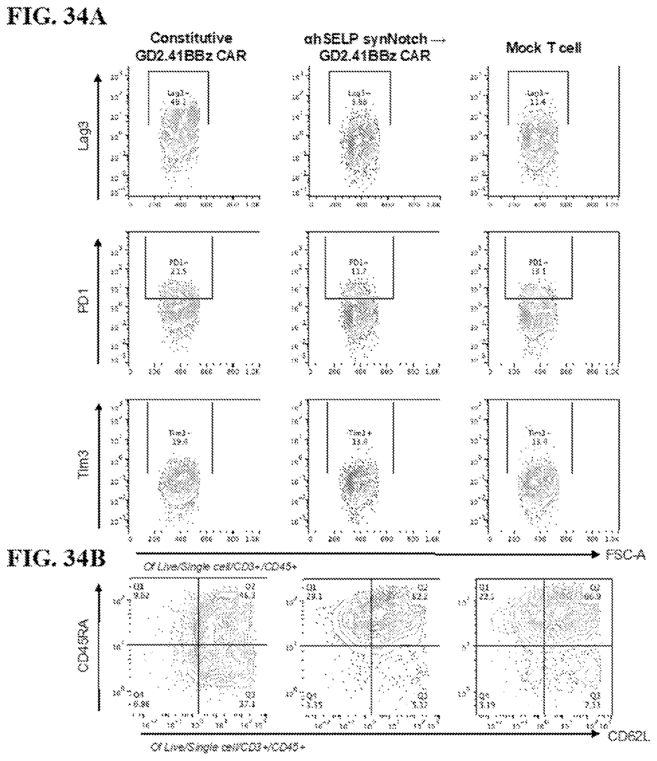

Next, we investigated the phenotypic and functional consequences of conditional CAR expression on T cell fitness. We found that resting (unstimulated) synNotch CAR T cells expressed significantly fewer exhaustion markers (Tim3, Lag3, and PD1) and maintained a more naïve-like phenotype (CD62L⁺, CD45RA⁺, and CD45RO⁻) seven days post-manufacture, as compared to constitutive CAR T cells (FIG. 20A-20B and FIGS. 34A-34B). These data suggest that eliminating CAR expression during manufacturing via controlled synNotch gating may prevent premature differentiation and exhaustion in pre-infusion products.

Despite these phenotypic differences, there was no significant change in cytotoxicity when unstimulated effector cells were co-cultured with target cells (FIG. 20C). To better understand CAR T cell function via serial killing of target cells, we set up a chronic stimulation assay where we stimulated effector cells with target cells for 15 days, periodically refreshing target cells for a total of four stimulations. After 15 days coincubation we found an increase in exhaustion marker expression on synNotch T cells to reach the same level as the constitutive CAR (FIG. 20D). Further, synNotch CAR T cells retained a less differentiated phenotype, as observed in the resting MEAT cells (FIG. 20E). We then performed a killing assay using chronically activated effector cells and found that synNotch CAR T cells maintained cytotoxic function, whereas constitutive CAR killing was completely abolished (FIG. 20F). These data suggest that exhaustion marker expression is not fully predictive of T cell function, and that other factors may influence cytotoxicity.

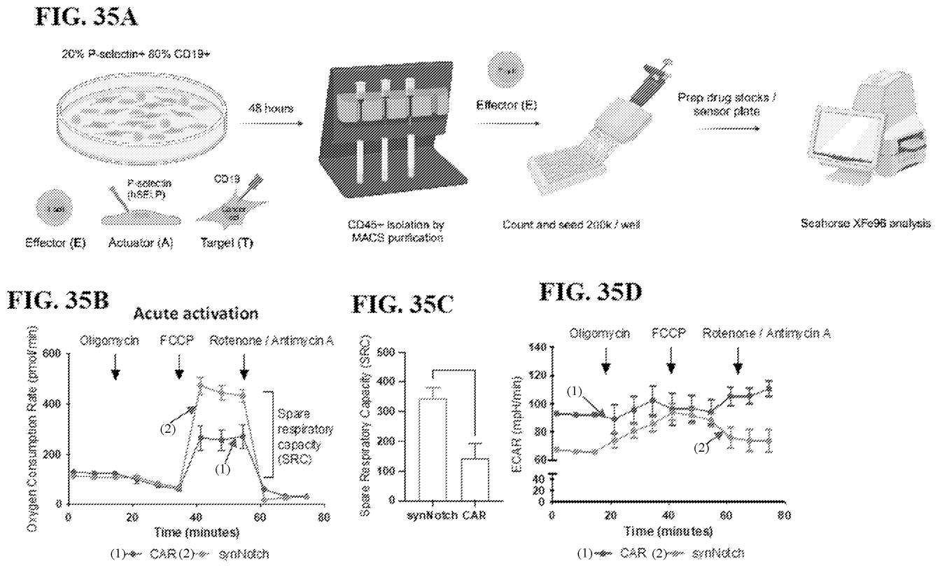

Given that MEAT cells maintain a less differentiated phenotype, and T cell differentiation is strongly interconnected to metabolism, we next investigated the impact of synNotch gating T cell bioenergetics. We used the Seahorse mitochondrial stress test to probe changes in oxygen consumption rate (OCR, a measure of oxidative phosphorylation) and extracellular acidification rate (ECAR, a measure of glycolysis) in resting (unstimulated), acutely (48 h) activated, and chronically (15 d) activated effector cells. CAR T cells were assayed for mitochondrial fitness through serial additions of oligomycin (complex V ATP synthase inhibitor), FCCP (uncoupler of oxidative phosphorylation) and rotenone and antimycin A (complex I & III inhibitors). Resting synNotch CAR T cells exhibited a significantly higher maximal OCR post-FCCP addition, as compared to resting constitutive CAR T cells (FIG. 20G). Similarly, chronically activated synNotch T cells had a significantly higher maximal OCR than chronically activated constitutive CAR T cells. This translated to a significantly higher spare respiratory capacity (SRC) in synNotch T cells, demonstrating their higher metabolic fitness in both resting and chronically stimulated T cell conditions (FIG. 20H). Similar trends were observed in acutely activated effector cells (FIGS. 35A-35D). We simultaneously assessed changes in ECAR, a measurement of bulk acidification due to changes in glycolysis and/or the oxidative phosphorylation. At baseline, resting synNotch T cells had the lowest ECAR, indicating minimal bioenergetic activity (FIG. 20I). These data support the observed less-differentiated memory phenotype of resting and chronically stimulated MEAT cells and suggest that conditional CAR expression may promote more favorable bioenergetics, improving cell metabolic fitness and function.

### Example 10: αP-Selectin synNotch GD2 CAR T Cells Ameliorate Neurotoxicity in Solid Tumor Models

Assessing P-selectin-specific killing in vivo would require a synNotch gated CAR T cell that recognizes murine P-selectin in TME blood vessels and GD2 on the human xenograft cells. Though extensive efforts were made to generate an anti-mouse P-selectin synNotch, the four published scFv sequences we were able to obtain only actuated in response to human P-selectin and did not cross-react with murine P-selectin. See, R. Nolo et al., Targeting P-selectin blocks neuroblastoma growth. Oncotarget 8, 86657-86670 (2017); K. I. Ataga et al., Use of the anti-P-Selectin antibody Crizanlizumab for treating sickle cell nephropathy and chronic kidney disease associated with sickle cell disease. World Intellectual Property Organization International Bureau WO 2021/024220 A1, (2021); K. I. Ataga et al., Crizanlizumab for the Prevention of Pain Crises in Sickle Cell Disease. N Engl J Med 376, 429-439 (2017); G. Hariri et al., Radiation-guided P-selectin antibody targeted to lung cancer. Ann Biomed Eng 36, 821-830 (2008); R. P. McEver, R. Alvarez, Z. Kawar, Anti-P-Selectin antibodies and methods of using the same to treat inflammlatory diseases. United States Patent and Trademark Office U.S. Pat. No. 8,377,440 B2, (2013); and D. Hallahan, R. Mernaugh, Phage antibodies to radiation-inducible neoantigens. United States Patent and Trademark Office U.S. Pat. No. 8,927,288 B2, (2015). Further optimization of heavy and light chain sequence order, linker length, and generation of Fab constructs from scFv did not improve actuation to murine P-selectin in primary T cells.

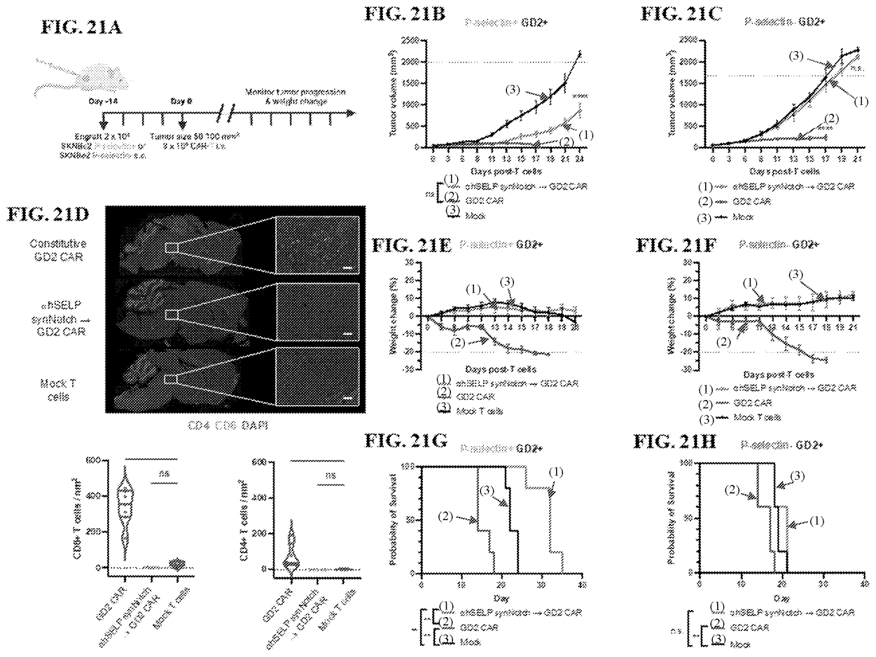

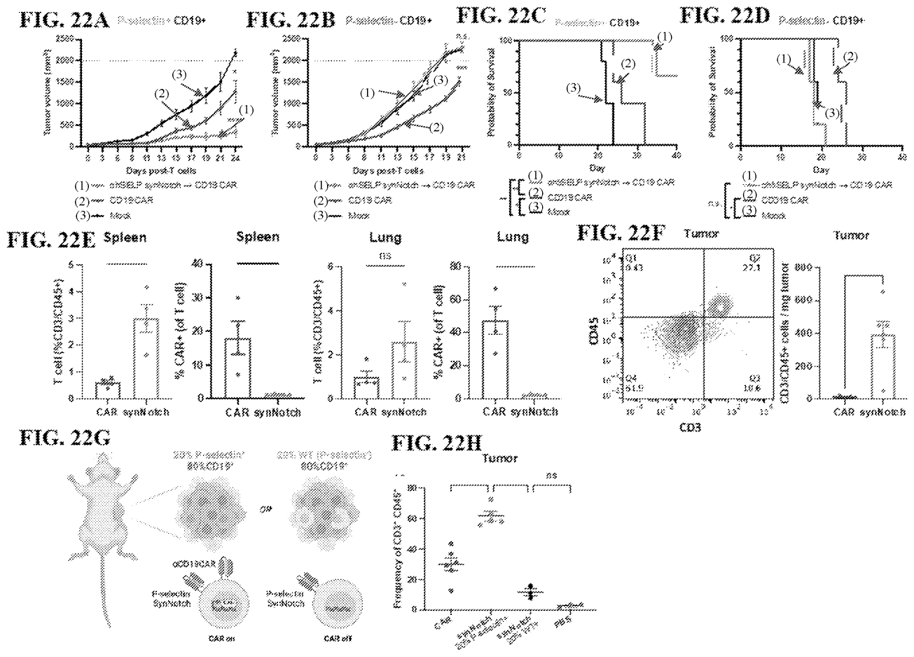

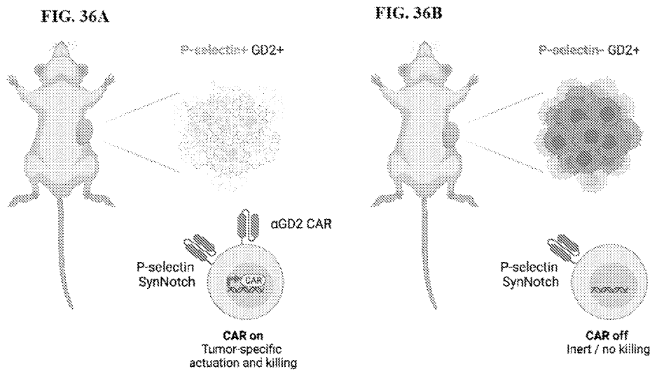

Therefore, we constructed a xenograft model that would mimic the in vivo gating system by stably expressing human P-selectin on the target neuroblastoma cells. We engrafted 2 million GD2⁺ SK-N-BE(2) neuroblastoma cells in the flank of NSG mice with one cohort expressing P-selectin and another cohort P-selectin null to model cis synNotch CAR T cell killing (FIG. 21A, FIGS. 36A-36B). We hypothesized that αP-selectin synNotch GD2 CAR T cells would actuate in the TME of P-selectin⁺ tumors and cause target cell killing without trafficking and toxicity in the brain. Furthermore, we postulated that the synNotch CAR T cells would be inert in the absence of P-selectin and have no impact on tumor growth.

In P-selectin⁺ tumors, αhSELP synNotch→GD2 CAR T cells led to a significant delay in tumor growth, relative to control mock T cell treated mice (FIG. 21B). Importantly, there was no significant change in tumor growth for synNotch treated mice in the P-selectin negative cohort (FIG. 21C). This suggests that there was no “leakage”, or non-specific CAR expression in the absence of actuator ligand. While we found significant infiltration of CD4⁺ and CD8⁺ T cells in constitutive GD2 CAR treated mice, there was no detectable T cell infiltration in the αhSELP synNotch→GD2 CAR T cell cohort (FIG. 21D). As previously observed, constitutive GD2 CAR treated mice had to be euthanized due to rapid weight loss (>20%) (FIGS. 21E-21F). Conversely, there was no observed weight loss in αhSELP synNotch→GD2 CAR T cell treated mice, suggesting a lack of systemic toxicity.

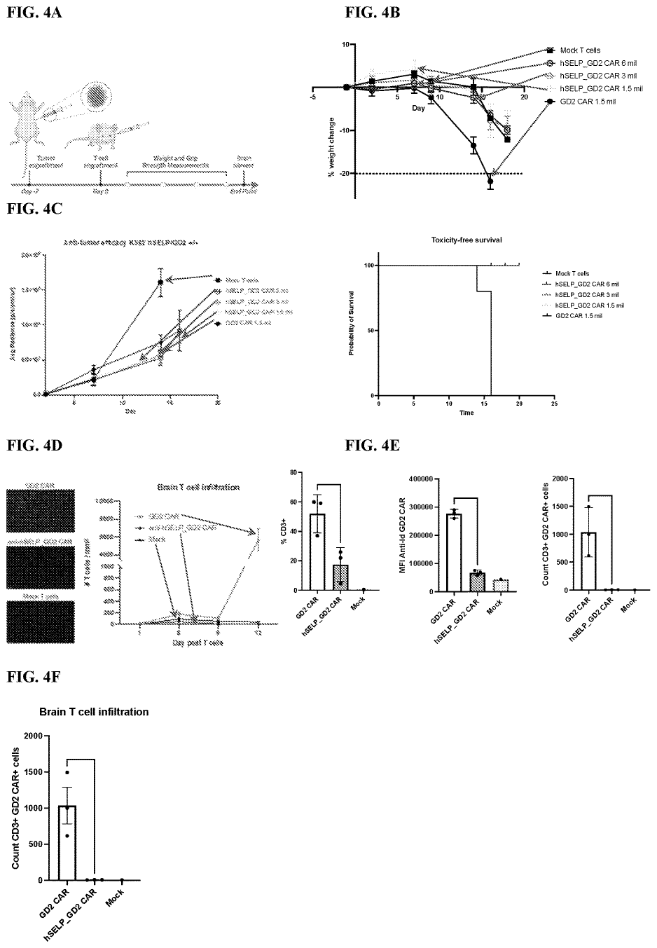

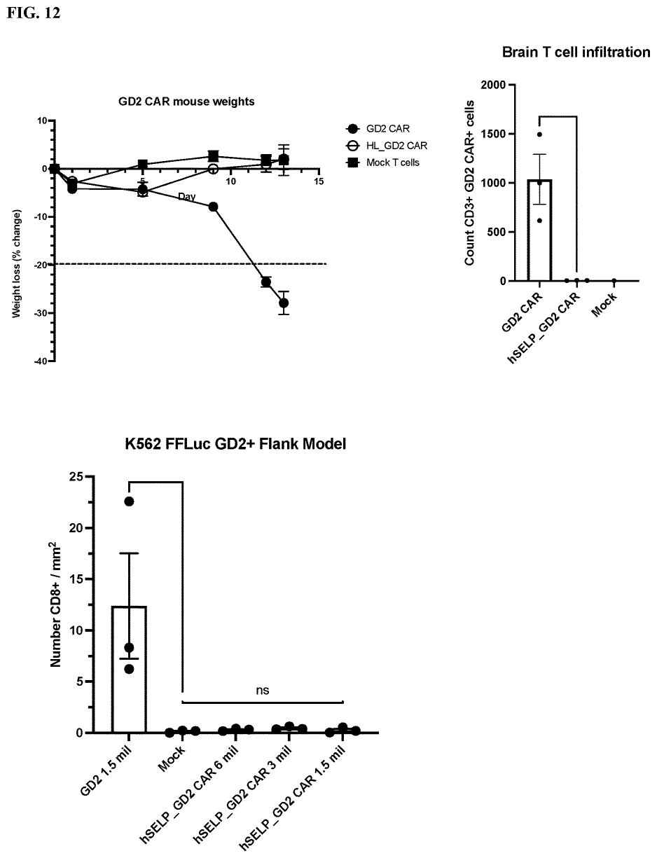

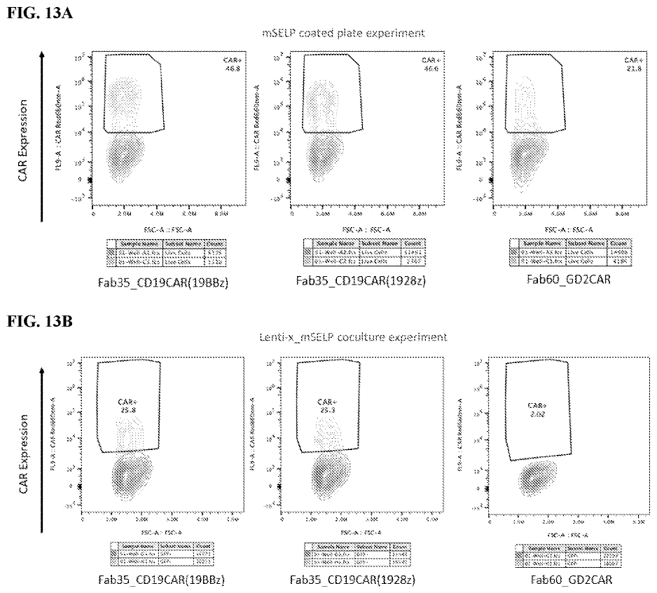

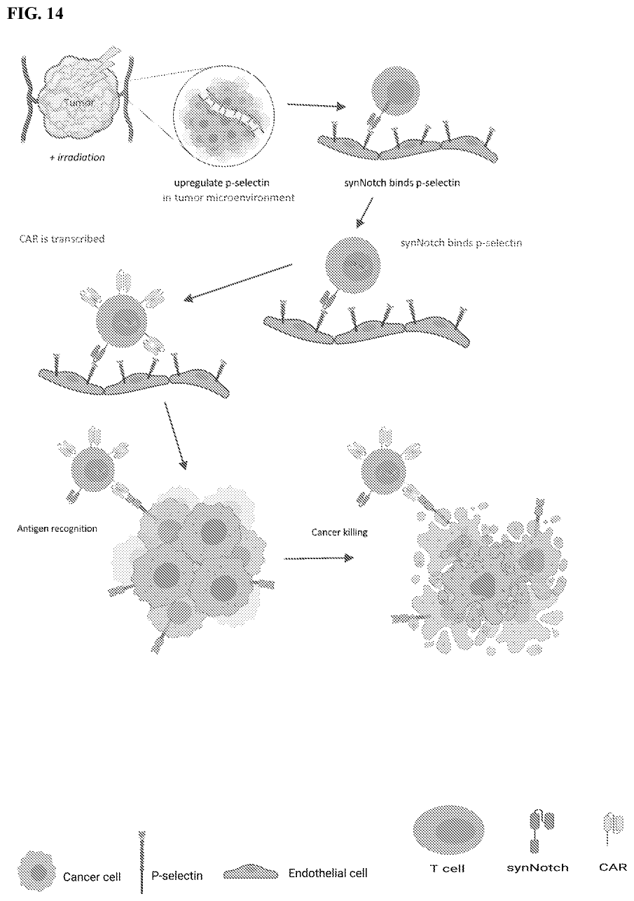

All MEAT cell treated mice were ultimately euthanized due to tumor burden (>2000 mm³) and not because of weight loss or signs of neurological distress, demonstrating P-selectin gated CAR T cells can ameliorate on-target off-tumor neurotoxicity (FIGS. 21G-21H). As predicted, in mice bearing P-selectin negative tumors, there was no survival benefit provided by any CAR T cell because the gated cells did not actuate to kill and the conventional GD2 CAR T cells killed the mice from toxicity more quickly than the control mice died from tumor burden. Similar trends were observed in a second solid tumor model where αhSELP synNotch→GD2 CAR T cells delayed K562 tumor growth and prevented T cell infiltration in the brain (FIGS. 4A-4F and FIG. 12).

### Example 11: αP-Selectin synNotch CD19 CAR T Cells Improve Anti-Tumor Efficacy in Solid Tumor Models

Finally, we sought to validate the MEAT cell system using a second CAR and solid tumor model. We engrafted NSG mice with SK-N-BE(2) neuroblastoma target cells stably expressing CD19 as previously described. Mice were treated with constitutive CD19 CAR T cells, αhSELP synNotch→CD19 CAR T cells or mock T cells and tumor growth kinetics were monitored by digital caliper. We observed similar trends as reported with the GD2 CAR, where P-selectin⁺ tumors had a significant delay in tumor growth in mice treated with gated CAR T cells and there was no therapeutic efficacy against P-selectin tumors relative to mock T cell control treated mice (FIGS. 22A-22B). Interestingly, the αhSELP synNotch→CD19 CAR T cell treated mice performed better than the constitutive CD19 CAR, leading to greater reductions in tumor sizes and a significant increase in overall survival in tumors that actuated the CAR (FIG. 22C). No benefit to survival or tumor reduction was observed in mice treated with the gated CAR T cells if P-selectin was absent (FIG. 22D).

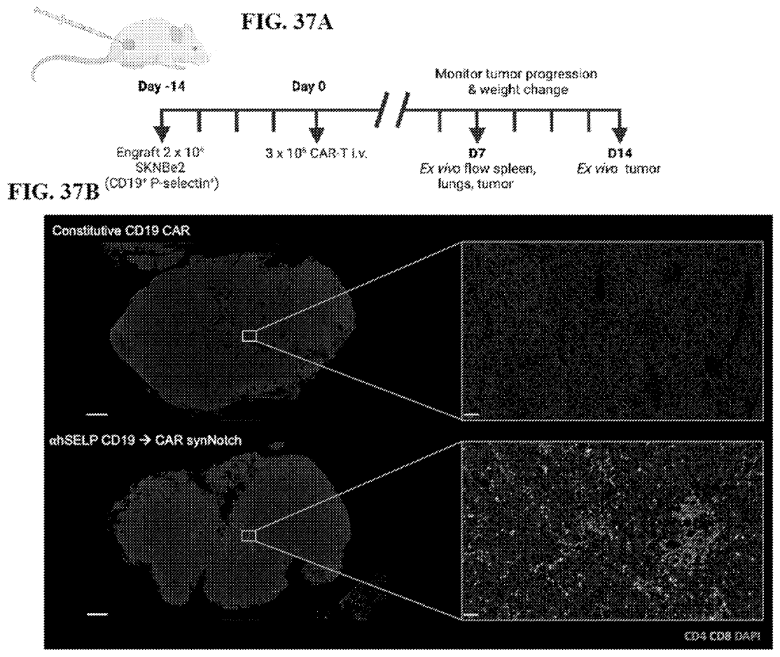

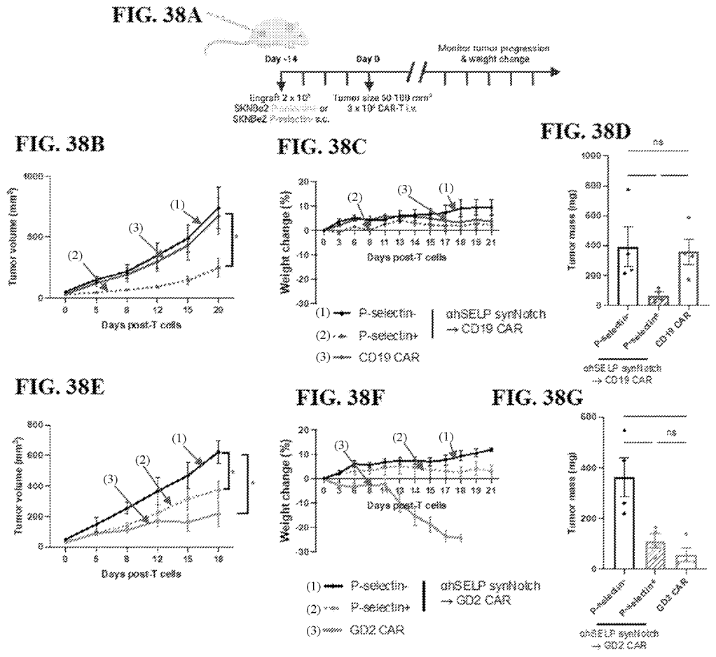

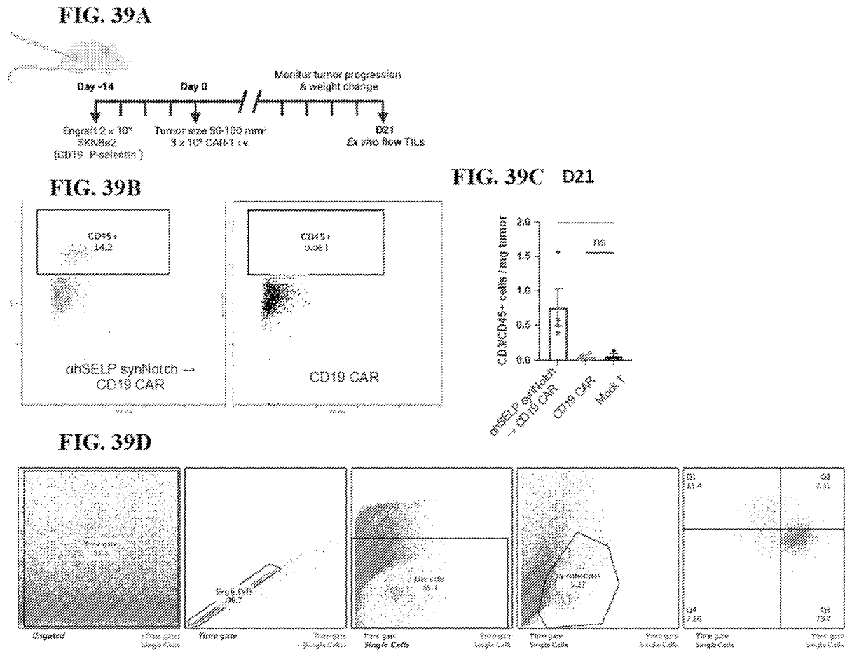

To further elucidate the mechanism of improved anti-tumor efficacy, we engrafted a second cohort of neuroblastoma tumor-bearing mice and assessed T cell infiltration on days 7 and 14 post-engraftment in the spleen, lungs and tumor by ex vivo flow cytometry (FIG. 37A). We found a significantly higher population of CD3+CD45+ T cells engrafting the spleen and lungs 7 days post-treatment in synNotch T cell-treated mice (FIGS. 22E-22F). Importantly, of the synNotch T cells detected in normal secondary lymphoid tissues, there was no detectable CAR expression compared to a mock T cell-stained control, demonstrating synNotch T cells in the “off state” outside of the TME. We found that αhSELP synNotch→CD19 CAR T cells persisted longer and had significantly higher infiltration of T cells in the tumor relative to the constitutive CD19 CAR, which was virtually non-detectable in the tumor by both flow cytometry and IF (FIG. 22E, FIG. 37B). Similar trends in anti-tumor efficacy and TIL quantification were observed with a second T cell donor using the same neuroblastoma xenograft model (FIGS. 38A-38G and FIGS. 39A-39D).

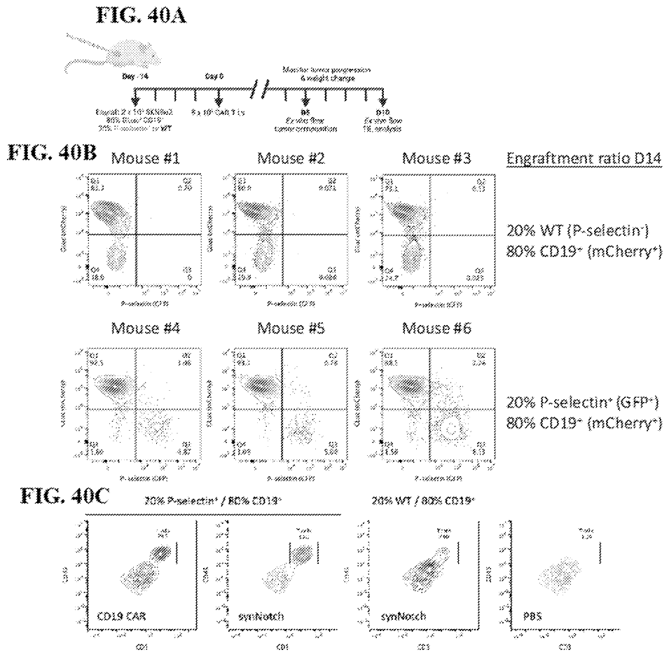

Finally, we established a mixed antigen model to probe trans synNotch CAR T cell killing, simulating an environment where P-selectin is present on normal antigen negative cells and the CAR target is present on cancer cells (FIG. 22G). We engrafted 2 million SK-N-BE(2) neuroblastoma cells in the flank of NSG mice with 20% of the total cells expressing P-selectin and 80% expressing CD19 (P-selectin/CD19). A separate cohort of control mice was engrafted with 20% WT SK-N-BE(2) negative for P-selectin and 80% expressing CD19 (WT/CD19). Two weeks post-tumor engraftment mice were dosed with either 5 million constitutive CD19 CAR T cells or αhSELP synNotch→CD19 CAR T cells. Tumor composition was verified by ex vivo flow cytometry 19 days post-tumor engraftment to ensure the initial antigen ratio was maintained. We found that the WT/CD19 mixed model maintained a 20/80% ratio, while the P-selectin/CD19 tumors decreased from 20% to 6% P-selectin⁺, possibly due to differential growth kinetics of target cells stably expressing P-selectin (FIGS. 40A-40C). We then harvested tumors, enriched for T cells, and assessed infiltration 10 days post-treatment by flow cytometry. We found that synNotch CAR T cells had significantly higher tumor infiltration and persistence, compared to the constitutive CD19 CAR, as observed in the cis killing model (FIG. 22H, FIG. 40C). Importantly, there was no significant increase in synNotch T cell infiltration in control WT/CD19 mice, demonstrating specific biodistribution and expansion to P-selectin+ tissue, even when the actuator target is present on a small fraction of the total cells. These data suggest that actuating via normal cells in the TME is a viable approach to gating toxic CAR T cells and that conditional CAR expression can improve cell fitness and function.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. An engineered T cell comprising
a. a nucleic acid encoding a P-selectin-specific chimeric receptor polypeptide comprising
i. an extracellular domain that specifically binds P-selectin;
ii. a heterologous receptor polypeptide comprising one or more ligand-inducible proteolytic cleavage sites; and
iii. an intracellular domain comprising a transcriptional activator, wherein binding of the extracellular domain to P-selectin induces cleavage of the heterologous receptor polypeptide at the one or more ligand-inducible proteolytic cleavage sites to release the intracellular domain, and
b. a nucleic acid encoding a chimeric antigen receptor (CAR) polypeptide that is operably linked to a promoter that is responsive to the transcriptional activator of the released intracellular domain of the P-selectin-specific chimeric receptor polypeptide.

2. The engineered T cell of claim 1, wherein the extracellular domain of the P-selectin-specific chimeric receptor polypeptide comprises an anti-P-selectin antigen binding fragment or a PSGL-1 polypeptide, optionally wherein the anti-P-selectin antigen binding fragment is a Fab, F(ab′)₂, or a scFv and/or wherein the PSGL-1 polypeptide comprises an amino acid sequence having at least 90%, at least 95%, at least 98%, at least 99%, or 100%, amino acid sequence identity to SEQ ID NO: 92.

3. (canceled)

4. (canceled)

5. The engineered T cell of claim 1, wherein the extracellular domain of the P-selectin specific chimeric receptor polypeptide comprises a heavy chain variable (V_{H}) region and a light chain variable (V_{L}) region, wherein
a. the V_{H} region comprises a V_{H} CDR1 sequence comprising SYDIN (SEQ ID NO: 63), a V_{H} CDR2 sequence comprising WIYPGDGSIKYNEKFKG (SEQ ID NO: 64), and a V_{H} CDR3 sequence comprising RGEYGNYEGAMDY (SEQ ID NO: 65); and the V_{L} region comprises a V_{L} CDR1 sequence comprising KASQSVDYDGHSYMN (SEQ ID NO: 66), a V_{L} CDR2 sequence comprising AASNLES (SEQ ID NO: 67), and a V_{L} CDR3 sequence comprising QQSDENPLT (SEQ ID NO: 68); or
b. the V_{H} region comprises a V_{H} CDR1 sequence comprising GYTFTSY (SEQ ID NO: 69), a V_{H} CDR2 sequence comprising DPYYGG (SEQ ID NO: 70), and a V_{H} CDR3 sequence comprising WDGYYGGFSY (SEQ ID NO: 71); and the V_{L} region comprises a V_{L} CDR1 sequence comprising RASSNVKYMY (SEQ ID NO: 72), a V_{L} CDR2 sequence comprising YTSNLAS (SEQ ID NO: 73), and a V_{L} CDR3 sequence comprising QQFTSSPYT (SEQ ID NO: 74); or
c. the V_{H} region comprises a V_{H} CDR1 sequence comprising GYSITSDY (SEQ ID NO: 75), a V_{H} CDR2 sequence comprising SSGR (SEQ ID NO: 76), and a V_{H} CDR3 sequence comprising HYGNYEGYYYAMDY (SEQ ID NO: 77); and/or
the V_{L} region comprises a V_{L} CDR1 sequence comprising ITSTGIDDDMN (SEQ ID NO: 78), a V_{L} CDR2 sequence comprising EGNVLRP (SEQ ID NO: 79), and a V_{L} CDR3 sequence comprising LQTDNLPLT (SEQ ID NO: 80);
optionally wherein the extracellular domain of the P-selectin specific chimeric receptor polypeptide comprises a V_{H} amino acid sequence selected from the group consisting of; SEQ ID NO: 81, SEQ ID NO: 83, and SEQ ID NO: 85; and a V_{L} amino acid sequence selected from the group consisting of; SEQ ID NO: 82; SEQ ID NO: 84; and SEQ ID NO: 86.

6. (canceled)

7. The engineered T cell of claim 1, wherein
the chimeric receptor polypeptide is expressed on the surface of the engineered T cell; or
the heterologous receptor polypeptide comprises a Notch receptor polypeptide, optionally wherein the Notch receptor polypeptide comprises an S2 proteolytic cleavage site and an S3 proteolytic cleavage site and/or wherein the Notch receptor polypeptide comprises the amino acid sequence of SEQ ID NO: 42 or SEQ ID NO: 43; or
the nucleic acid encoding the P-selectin-specific chimeric receptor polypeptide and/or the nucleic acid encoding the CAR polypeptide comprises a signal peptide sequence; or
the transcriptional activator comprises Gal4-VP16; or
the nucleic acid encoding the P-selectin-specific chimeric receptor polypeptide is operably linked to a promoter, optionally wherein the promoter is a constitutive promoter; or
the CAR polypeptide comprises (i) an extracellular antigen binding domain: (ii) a transmembrane domain; and (iii) an intracellular domain; or
the T cell is a CD4+ T cell or a CD8+ T cell; or
the T cell is derived from an autologous donor or an allogenic donor.

8. (canceled)

9. (canceled)

10. (canceled)

11. (canceled)

12. (canceled)

13. (canceled)

14. (canceled)

15. The engineered T cell of claim 7, wherein
the extracellular antigen binding domain binds to a target antigen, optionally wherein the target antigen is a tumor antigen and optionally wherein the target antigen is CD19 or GD2; or
the extracellular antigen binding domain comprises a single chain variable fragment (scFv); or
the extracellular antigen binding domain comprises a GD2 scFv having at least 80%, 85%, 90%, 95%, 96%, 97%, 98%, or 99% sequence identity to SEQ ID NO: 3, optionally wherein the extracellular antigen binding domain comprises a GD2 scFv of SEQ ID NO: 3; or
the transmembrane domain comprises a CD8 transmembrane domain; or
the intracellular domain of the CAR polypeptide comprises one or more costimulatory domains, optionally wherein the one or more costimulatory domains are selected from a CD28 costimulatory domain, a CD3ζ chain, a 4-1BBL costimulatory domain, or any combination thereof.

16. (canceled)

17. (canceled)

18. (canceled)

19. (canceled)

20. (canceled)

21. (canceled)

22. (canceled)

23. (canceled)

24. (canceled)

25. A method for treating cancer in a subject in need thereof comprising administering to the subject an effective amount of the engineered T cells of claim 1.

26. The method of claim 25, wherein
the engineered T cells are administered intravenously, intraperitoneally, subcutaneously, intramuscularly, or intratumorally; or
the cancer is a carcinoma, sarcoma, a melanoma, or a hematopoietic cancer; or
the cancer is selected from among adrenal cancers, bladder cancers, blood cancers, bone cancers, brain cancers, breast cancers, carcinoma, cervical cancers, colon cancers, colorectal cancers, corpus uterine cancers, ear, nose and throat (ENT) cancers, endometrial cancers, esophageal cancers, gastrointestinal cancers, head and neck cancers, Hodgkin's disease, intestinal cancers, kidney cancers, larynx cancers, leukemias, liver cancers, lymph node cancers, lymphomas, lung cancers, melanomas, mesothelioma, myelomas, nasopharynx cancers, neuroblastomas, non-Hodgkin's lymphoma, oral cancers, ovarian cancers, pancreatic cancers, penile cancers, pharynx cancers, prostate cancers, rectal cancers, sarcoma, seminomas, skin cancers, stomach cancers, teratomas, testicular cancers, thyroid cancers, uterine cancers, vaginal cancers, vascular tumors, and metastases thereof; or
the method further comprises administering to the subject an additional cancer therapy, optionally wherein the additional cancer therapy is selected from among chemotherapy, radiation therapy, immunotherapy, monoclonal antibodies, anticancer nucleic acids or proteins, anti-cancer viruses or microorganisms, and any combinations thereof; and/or
the method further comprises administering a cytokine to the subject, optionally wherein the cytokine is administered prior to, during, or subsequent to administration of the one or more engineered T cells and/or wherein the cytokine is selected from a group consisting of interferon a, interferon β, interferon γ, complement C5a, IL-2, TNF alpha, CD40L, IL12, IL-23, IL15, IL17, CCL1, CCL11, CCL12, CCL13, CCL14-1, CCL14-2, CCL14-3, CCL15-1, CCL15-2, CCL16, CCL17, CCL18, CCL19, CCL19, CCL2, CCL20, CCL21, CCL22, CCL23-1, CCL23-2, CCL24, CCL25-1, CCL25-2, CCL26, CCL27, CCL28, CCL3, CCL3L1, CCL4, CCL4L1, CCL5, CCL6, CCL7, CCL8, CCL9, CCR10, CCR2, CCR5, CCR6, CCR7, CCR8, CCRL1, CCRL2, CX3C_{L}1, CX3CR, CXCL1, CXCL10, CXCL11, CXCL12, CXCL13, CXCL14, CXCL15, CXCL16, CXCL2, CXCL3, CXCL4, CXCL5, CXCL6, CXCL7, CXCL8, CXCL9, CXCL9, CXCR1, CXCR2, CXCR4, CXCR5, CXCR6, CXCR7 and XCL2.

27. (canceled)

28. (canceled)

29. (canceled)

30. (canceled)

31. (canceled)

32. (canceled)

33. (canceled)

34. A method for mitigating toxicities associated with CAR T cell therapy in a subject in need thereof comprising administering an effective amount of the engineered T cells of claim 1.

35. A vector comprising
a nucleic acid encoding a P-selectin-specific chimeric receptor polypeptide comprising
i. an extracellular domain that specifically binds P-selectin;
ii. a heterologous receptor polypeptide comprising one or more ligand-inducible proteolytic cleavage sites; and
iii. an intracellular domain comprising a transcriptional activator, wherein binding of the extracellular domain to P-selectin induces cleavage of the heterologous receptor polypeptide at the one or more ligand-inducible proteolytic cleavage sites to release the intracellular domain, and
a nucleic acid encoding a chimeric antigen receptor (CAR) polypeptide that is operably linked to a promoter that is responsive to the transcriptional activator of the released intracellular domain of the P-selectin-specific chimeric receptor polypeptide.

36. The vector of claim 35, wherein
the vector is a viral vector, a retroviral vector or a plasmid; or
the nucleic acid encoding the P-selectin-specific chimeric receptor polypeptide is operably linked to a promoter, optionally wherein the promoter is a constitutive promoter; or
the extracellular domain of the P-selectin-specific chimeric receptor polypeptide comprises an anti-P-selectin antigen binding fragment or a PSGL-1 polypeptide;
optionally wherein the PSGL-1 polypeptide comprises an amino acid sequence having at least 90%, at least 95%, at least 98%, at least 99%, or 100%, amino acid sequence identity to SEQ ID NO: 92; or
the heterologous receptor polypeptide comprises a Notch receptor polypeptide, optionally wherein the Notch receptor polypeptide comprises an S2 proteolytic cleavage site and an S3 proteolytic cleavage site and/or wherein the Notch receptor polypeptide comprises the amino acid sequence of SEQ ID NO: 42 or SEQ ID NO: 43:
the transcriptional activator comprises Gal4-VP16; or
the nucleic acid encoding the P-selectin-specific chimeric receptor polypeptide is operably linked to a promoter, optionally wherein the promoter is a constitutive promoter.

37. (canceled)

38. (canceled)

39. The vector of claim 35, wherein the extracellular domain of the P-selectin specific chimeric receptor polypeptide comprises a heavy chain variable (V_{H}) region and a light chain variable (V_{L}) region, wherein
a. the V_{H} region comprises a V_{H} CDR1 sequence comprising SYDIN (SEQ ID NO: 63), a V_{H} CDR2 sequence comprising WIYPGDGSIKYNEKFKG (SEQ ID NO: 64), and a V_{H} CDR3 sequence comprising RGEYGNYEGAMDY (SEQ ID NO: 65); and the V_{L} region comprises a V_{L} CDR1 sequence comprising KASQSVDYDGHSYMN (SEQ ID NO: 66), a V_{L} CDR2 sequence comprising AASNLES (SEQ ID NO: 67), and a V_{L} CDR3 sequence comprising QQSDENPLT (SEQ ID NO: 68); or
b. the V_{H} region comprises a V_{H} CDR1 sequence comprising GYTFTSY (SEQ ID NO: 69), a V_{H} CDR2 sequence comprising DPYYGG (SEQ ID NO: 70), and a V_{H} CDR3 sequence comprising WDGYYGGFSY (SEQ ID NO: 71); and the V_{L} region comprises a V_{L} CDR1 sequence comprising RASSNVKYMY (SEQ ID NO: 72), a V_{L} CDR2 sequence comprising YTSNLAS (SEQ ID NO: 73), and a V_{L} CDR3 sequence comprising QQFTSSPYT (SEQ ID NO: 74); or
c. the V_{H} region comprises a V_{H} CDR1 sequence comprising GYSITSDY (SEQ ID NO: 75), a V_{H} CDR2 sequence comprising SSGR (SEQ ID NO: 76), and a V_{H} CDR3 sequence comprising HYGNYEGYYYAMDY (SEQ ID NO: 77); and/or the V_{L} region comprises a V_{L} CDR1 sequence comprising ITSTGIDDDMN (SEQ ID NO: 78), a V_{L} CDR2 sequence comprising EGNVLRP (SEQ ID NO: 79), and a V_{L} CDR3 sequence comprising LQTDNLPLT (SEQ ID NO: 80);
or wherein the extracellular domain of the P-selectin specific chimeric receptor polypeptide comprises a V_{H} amino acid sequence selected from the group consisting of; SEQ ID NO: 81, SEQ ID NO: 83, and SEQ ID NO: 85; and a V_{L} amino acid sequence selected from the group consisting of; SEQ ID NO: 82; SEQ ID NO: 84; and SEQ ID NO: 86.

40. (canceled)

41. (canceled)

42. (canceled)

43. (canceled)

44. (canceled)

45. (canceled)

46. (canceled)

47. The vector of claim 35, wherein the CAR polypeptide comprises (i) an extracellular antigen binding domain; (ii) a transmembrane domain;
and (iii) an intracellular domain.

48. The vector of claim 47, wherein the extracellular antigen binding domain comprises a single chain variable fragment (scFv).

49. The vector of claim 47, wherein the extracellular antigen binding domain binds to a target antigen, optionally wherein the target antigen is a tumor antigen.

50. The vector of claim 47, wherein the extracellular antigen binding domain comprises a GD2 scFv of SEQ ID NO: 3.

51. A method for preparing T cells for cancer therapy, comprising isolating T cells from a donor subject, and transducing the T cells with the vector of any of claim 35.

52. A method of treatment comprising isolating T cells from a donor subject, transducing the T cells with the vector of claim 35, and administering the transduced T cells to a recipient subject.

53. The method of claim 52, wherein the donor subject and the recipient subject are the same.

54. The method of claim 52, wherein the donor subject and the recipient subject are different.

55. The method of claim 54, wherein the T cell is a CD4+ T cell or a CD8+ T cell.


SEQUENCE LISTING (101 sequences)

SEQ ID NO: 1 - protein, 15 aa, synthetic construct
GGGGSGGGGSGGGGS

SEQ ID NO: 2 - DNA, 45 bp, synthetic construct
  source 1..45: mol_type = other DNA
ggcggcggcggatctggaggtggtggctcaggtggcggaggctcc

SEQ ID NO: 3 - protein, 279 aa, Homo sapiens
MEFGLSWLFLVAILKGVQCGSDILLTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKRADAAPTVSIFPGGGGSGGGGSGGGGSGGGGSEVKLQQSGPSLVEPGASVMISCKASGSSFTGYNMNWVRQNIGKSLEWIGAIDPYYGGTSYNQKFKGRATLTVDKSSSTAYMHLKSLTSEDSAVYYCVSGMKYWGQGTSVTVSS

SEQ ID NO: 4 - DNA, 525 bp, Homo sapiens
  source 1..525: mol_type = genomic DNA
tgataaggatctcgaggccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctcc

SEQ ID NO: 5 - protein, 75 aa, synthetic construct
  VARIANT 1..75: note = This sequence may encompass 1-15 GGGGS repeating units
GGGGSGGGGSGGGGSGGGGSGGGGSGGGGSGGGGSGGGGSGGGGSGGGGSGGGGSGGGGSGGGGSGGGGSGGGGS

SEQ ID NO: 6 - protein, 21 aa, Homo sapiens
MALPVTALLLPLALLLHAARP

SEQ ID NO: 7 - DNA, 63 bp, Homo sapiens
  source 1..63: mol_type = genomic DNA
atggccctgccagtaacggctctgctgctgccacttgctctgctcctccatgcagccaggcct

SEQ ID NO: 8 - protein, 18 aa, Homo sapiens
MALPVTALLLPLALLLHA

SEQ ID NO: 9 - DNA, 54 bp, Homo sapiens
  source 1..54: mol_type = genomic DNA
atggctctcccagtgactgccctactgcttcccctagcgcttctcctgcatgca

SEQ ID NO: 10 - protein, 21 aa, Homo sapiens
MEFGLSWLFLVAILKGVQCGS

SEQ ID NO: 11 - protein, 21 aa, Mus sp.
MASPLTRFLSLNLLLLGESII

SEQ ID NO: 12 - DNA, 63 bp, Mus sp.
  source 1..63: mol_type = genomic DNA
atggccagccccctgaccaggttcctgagcctgaacctgctgctgctgggcgagagcatcatc

SEQ ID NO: 13 - protein, 18 aa, Mus sp.
MASPLTRFLSLNLLLLGE

SEQ ID NO: 14 - DNA, 54 bp, Mus sp.
  source 1..54: mol_type = genomic DNA
atggccagccccctgaccaggttcctgagcctgaacctgctgctgctgggcgag

SEQ ID NO: 15 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 16 - DNA, 321 bp, Homo sapiens
  source 1..321: mol_type = genomic DNA
attgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 17 - protein, 234 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPWKSGDKPSLSARYV

SEQ ID NO: 18 - protein, 71 aa, Homo sapiens
PTTTPAPRPPTPAPTIASQPLSLRPEACRPAAGGAVHTRGLDFACDIYIWAPLAGTCGVLLLSLVITLYCN

SEQ ID NO: 19 - DNA, 213 bp, Homo sapiens
  source 1..213: mol_type = genomic DNA
cccaccacgacgccagcgccgcgaccaccaaccccggcgcccacgatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttactgcaac

SEQ ID NO: 20 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 21 - protein, 164 aa, Homo sapiens; identical to SEQ ID NO: 20

SEQ ID NO: 22 - protein, 112 aa, Homo sapiens
RVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 24 - DNA, 337 bp, synthetic construct
  source 1..337: mol_type = other DNA
agagtgaagttcagcaggagcgcagagccccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcg

SEQ ID NO: 25 - DNA, 339 bp, synthetic construct
  source 1..339: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 26 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 27 - protein, 42 aa, Homo sapiens
KRGRKKLLYIFKQPFMRPVQTTQEEDGCSCRFPEEEEGGCEL

SEQ ID NO: 28 - DNA, 126 bp, Homo sapiens
  source 1..126: mol_type = genomic DNA
aaacggggcagaaagaagctcctgtatatattcaaacaaccatttatgagaccagtacaaactactcaagaggaagatggctgtagctgccgatttccagaagaagaagaaggaggatgtgaactg

SEQ ID NO: 29 - protein, 277 aa, Homo sapiens
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 30 - protein, 199 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 31 - protein, 4 aa, synthetic construct
YVKM

SEQ ID NO: 32 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 33 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 34 - protein, 525 aa, Homo sapiens
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 35 - protein, 370 aa, Homo sapiens
MLGQVVTLILLLLLKVYQGKGCQGSADHVVSISGVPLQLQPNSIQTKVDSIAWKKLLPSQNGFHHILKWENGSLPSNTSNDRFSFIVKNLSLLIKAAQQQDSGLYCLEVTSISGKVQTATFQVFVFESLLPDKVEKPRLQGQGKILDRGRCQVALSCLVSRDGNVSYAWYRGSKLIQTAGNLTYLDEEVDINGTHTYTCNVSNPVSWESHTLNLTQDCQNAHQEFRFWPFLVIIVILSALFLGTLACFCVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 36 - protein, 289 aa, Homo sapiens
MKTLPAMLGTGKLFWVFFLIPYLDIWNIHGKESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYRLLPLGGLPLLITTCFCLFCCLRRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 37 - protein, 2555 aa, Homo sapiens
MPPLLAPLLCLALLPALAARGPRCSQPGETCLNGGKCEAANGTEACVCGGAFVGPRCQDPNPCLSTPCKNAGTCHVVDRRGVADYACSCALGFSGPLCLTPLDNACLTNPCRNGGTCDLLTLTEYKCRCPPGWSGKSCQQADPCASNPCANGGQCLPFEASYICHCPPSFHGPTCRQDVNECGQKPGLCRHGGTCHNEVGSYRCVCRATHTGPNCERPYVPCSPSPCQNGGTCRPTGDVTHECACLPGFTGQNCEENIDDCPGNNCKNGGACVDGVNTYNCRCPPEWTGQYCTEDVDECQLMPNACQNGGTCHNTHGGYNCVCVNGWTGEDCSENIDDCASAACFHGATCHDRVASFYCECPHGRTGLLCHLNDACISNPCNEGSNCDTNPVNGKAICTCPSGYTGPACSQDVDECSLGANPCEHAGKCINTLGSFECQCLQGYTGPRCEIDVNECVSNPCQNDATCLDQIGEFQCICMPGYEGVHCEVNTDECASSPCLHNGRCLDKINEFQCECPTGFTGHLCQYDVDECASTPCKNGAKCLDGPNTYTCVCTEGYTGTHCEVDIDECDPDPCHYGSCKDGVATFTCLCRPGYTGHHCETNINECSSQPCRHGGTCQDRDNAYLCFCLKGTTGPNCEINLDDCASSPCDSGTCLDKIDGYECACEPGYTGSMCNINIDECAGNPCHNGGTCEDGINGFTCRCPEGYHDPTCLSEVNECNSNPCVHGACRDSLNGYKCDCDPGWSGTNCDINNNECESNPCVNGGTCKDMTSGYVCTCREGFSGPNCQTNINECASNPCLNQGTCIDDVAGYKCNCLLPYTGATCEVVLAPCAPSPCRNGGECRQSEDYESFSCVCPTGWQGQTCEVDINECVLSPCRHGASCQNTHGGYRCHCQAGYSGRNCETDIDDCRPNPCHNGGSCTDGINTAFCDCLPGFRGTFCEEDINECASDPCRNGANCTDCVDSYTCTCPAGFSGIHCENNTPDCTESSCFNGGTCVDGINSFTCLCPPGFTGSYCQHDVNECDSQPCLHGGTCQDGCGSYRCTCPQGYTGPNCQNLVHWCDSSPCKNGGKCWQTHTQYRCECPSGWTGLYCDVPSVSCEVAAQRQGVDVARLCQHGGLCVDAGNTHHCRCQAGYTGSYCEDLVDECSPSPCQNGATCTDYLGGYSCKCVAGYHGVNCSEEIDECLSHPCQNGGTCLDLPNTYKCSCPRGTQGVHCEINVDDCNPPVDPVSRSPKCFNNGTCVDQVGGYSCTCPPGFVGERCEGDVNECLSNPCDARGTQNCVQRVNDFHCECRAGHTGRRCESVINGCKGKPCKNGGTCAVASNTARGFICKCPAGFEGATCENDARTCGSLRCLNGGTCISGPRSPTCLCLGPFTGPECQFPASSPCLGGNPCYNQGTCEPTSESPFYRCLCPAKFNGLLCHILDYSFGGGAGRDIPPPLIEEACELPECQEDAGNKVCSLQCNNHACGWDGGDCSLNFNDPWKNCTQSLQCWKYFSDGHCDSQCNSAGCLFDGFDCQRAEGQCNPLYDQYCKDHFSDGHCDQGCNSAECEWDGLDCAEHVPERLAAGTLVVVVLMPPEQLRNSSFHFLRELSRVLHTNVVFKRDAHGQQMIFPYYGREEELRKHPIKRAAEGWAAPDALLGQVKASLLPGGSEGGRRRRELDPMDVRGSIVYLEIDNRQCVQASSQCFQSATDVAAFLGALASLGSLNIPYKIEAVQSETVEPPPPAQLHFMYVAAAAFVLLFFVGCGVLLSRKRRRQHGQLWFPEGFKVSEASKKKRREPLGEDSVGLKPLKNASDGALMDDNQNEWGDEDLETKKFRFEEPVVLPDLDDQTDHRQWTQQHLDAADLRMSAMAPTPPQGEVDADCMDVNVRGPDGFTPLMIASCSGGGLETGNSEEEEDAPAVISDFIYQGASLHNQTDRTGETALHLAARYSRSDAAKRLLEASADANIQDNMGRTPLHAAVSADAQGVFQILIRNRATDLDARMHDGTTPLILAARLAVEGMLEDLINSHADVNAVDDLGKSALHWAAAVNNVDAAVVLLKNGANKDMQNNREETPLFLAAREGSYETAKVLLDHFANRDITDHMDRLPRDIAQERMHHDIVRLLDEYNLVRSPQLHGAPLGGTPTLSPPLCSPNGYLGSLKPGVQGKKVRKPSSKGLACGSKEAKDLKARRKKSQDGKGCLLDSSGMLSPVDSLESPHGYLSDVASPPLLPSPFQQSPSVPLNHLPGMPDTHLGIGHLNVAAKPEMAALGGGGRLAFETGPPRLSHLPVASGTSTVLGSSSGGALNFTVGGSTSLNGQCEWLSRLQSGMVPNQYNPLRGSVAPGPLSTQAPSLQHGMVGPLHSSLAASALSQMMSYQGLPSTRLATQPHLVQTQQVQPQNLQMQQQNLQPANIQQQQSLQPPPPPPQPHLGVSSAASGHLGRSFLSGEPSQADVQPLGPSSLAVHTILPQESPALPTSLPSSLVPPVTAAQFLTPPSQHSYSSPVDNTPSHQLQVPEHPFLTPSPESPDQWSSSSPHSNVSDWSEGVSSPPTSMQSQIARIPEAFK

SEQ ID NO: 38 - protein, 49 aa, synthetic construct
IPYKIEAVKSEPVEPPLPSQLHLMYVAAAAFVLLFFVGCGVLLSRKRRR

SEQ ID NO: 39 - protein, 120 aa, synthetic construct
PPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDC

SEQ ID NO: 40 - protein, 152 aa, synthetic construct
AAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLP

SEQ ID NO: 41 - protein, 23 aa, synthetic construct
HLMYVAAAAFVLLFFVGCGVLLS

SEQ ID NO: 42 - protein, 306 aa, synthetic construct
PPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLS

SEQ ID NO: 43 - protein, 321 aa, synthetic construct
ILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLS

SEQ ID NO: 44 - protein, 37 aa, synthetic construct
PCVGSNPCYNQGTCEPTSENPFYRCLCPAKFNGLLCH

SEQ ID NO: 45 - protein, 37 aa, synthetic construct
DINECVLSPCRHGASCQNTHGGYRCHCQAGYSGRNCE

SEQ ID NO: 46 - protein, 37 aa, synthetic construct
DIDDCRPNPCHNGGSCTDGINTAFCDCLPGFRGTFCE

SEQ ID NO: 47 - protein, 37 aa, synthetic construct
DINECASDPCRNGANCTDCVDSYTCTCPAGFSGIHCE

SEQ ID NO: 48 - protein, 32 aa, synthetic construct
TESSCFNGGTCVDGINSFTCLCPPGFTGSYCQ

SEQ ID NO: 49 - protein, 37 aa, synthetic construct
DVNECDSQPCLHGGTCQDGCGSYRCTCPQGYTGPNCQ

SEQ ID NO: 50 - protein, 27 aa, synthetic construct
DSSPCKNGGKCWQTHTQYRCECPSGWT

SEQ ID NO: 51 - protein, 36 aa, synthetic construct
LVDECSPSPCQNGATCTDYLGGYSCKCVAGYHGVNC

SEQ ID NO: 52 - protein, 36 aa, synthetic construct
IDECLSHPCQNGGTCLDLPNTYKCSCPRGTQGVHCE

SEQ ID NO: 53 - protein, 28 aa, synthetic construct
CFNNGTCVDQVGGYSCTCPPGFVGERCE

SEQ ID NO: 54 - protein, 39 aa, synthetic construct
DVNECLSNPCDARGTQNCVQRVNDFHCECRAGHTGRRCE

SEQ ID NO: 55 - protein, 10 aa, synthetic construct
GRRRRELDPM

SEQ ID NO: 56 - protein, 9 aa, synthetic construct
RQRRELDPM

SEQ ID NO: 57 - protein, 8 aa, synthetic construct
KIEAVKSE

SEQ ID NO: 58 - protein, 8 aa, synthetic construct
KIEAVQSE

SEQ ID NO: 59 - protein, 8 aa, synthetic construct
VGCGVLLS

SEQ ID NO: 60 - protein, 7 aa, synthetic construct
GCGVLLS

SEQ ID NO: 61 - protein, 205 aa, synthetic construct
LEIRAAFLRQRNTALRTEVAELEQEVQRLENEVSQYETRYGPLGGGKGGSGGSGGSMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAA

SEQ ID NO: 62 - protein, 211 aa, synthetic construct
MKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 63 - protein, 5 aa, synthetic construct
SYDIN

SEQ ID NO: 64 - protein, 17 aa, synthetic construct
WIYPGDGSIKYNEKFKG

SEQ ID NO: 65 - protein, 13 aa, synthetic construct
RGEYGNYEGAMDY

SEQ ID NO: 66 - protein, 15 aa, synthetic construct
KASQSVDYDGHSYMN

SEQ ID NO: 67 - protein, 7 aa, synthetic construct
AASNLES

SEQ ID NO: 68 - protein, 9 aa, synthetic construct
QQSDENPLT

SEQ ID NO: 69 - protein, 7 aa, synthetic construct
GYTFTSY

SEQ ID NO: 70 - protein, 6 aa, synthetic construct
DPYYGG

SEQ ID NO: 71 - protein, 10 aa, synthetic construct
WDGYYGGFSY

SEQ ID NO: 72 - protein, 10 aa, synthetic construct
RASSNVKYMY

SEQ ID NO: 73 - protein, 7 aa, synthetic construct
YTSNLAS

SEQ ID NO: 74 - protein, 9 aa, synthetic construct
QQFTSSPYT

SEQ ID NO: 75 - protein, 8 aa, synthetic construct
GYSITSDY

SEQ ID NO: 76 - protein, 4 aa, synthetic construct
SSGR

SEQ ID NO: 77 - protein, 14 aa, synthetic construct
HYGNYEGYYYAMDY

SEQ ID NO: 78 - protein, 11 aa, synthetic construct
ITSTGIDDDMN

SEQ ID NO: 79 - protein, 7 aa, synthetic construct
EGNVLRP

SEQ ID NO: 80 - protein, 9 aa, synthetic construct
LQTDNLPLT

SEQ ID NO: 81 - protein, 122 aa, synthetic construct
QVQLVQSGAEVKKPGASVKVSCKVSGYTFTSYDINWVRQAPGKGLEWMGWIYPGDGSIKYNEKFKGRVTMTVDKSTDTAYMELSSLRSEDTAVYYCARRGEYGNYEGAMDYWGQGTLVTVSS

SEQ ID NO: 82 - protein, 112 aa, synthetic construct
DIQMTQSPSSLSASVGDRVTITCKASQSVDYDGHSYMNWYQQKPGKAPKLLIYAASNLESGVPSRFSGSGSGTDFTLTISSLQPEDFATYYCQQSDENPLTFGGGTKVEIKR

SEQ ID NO: 83 - protein, 121 aa, synthetic construct
MAQVKLQQSGPELVKPGASVKMSCKASGYTFTSYVMHWVKQSNGKSLEWIGTIDPYYGGTSYNQKFKGKATLTVDKSSTTAYIQLKSLTSEDSAVYYCARWDGYYGGFSYWGQGTMVTVSS

SEQ ID NO: 84 - protein, 125 aa, synthetic construct
DIELTQSPAIMSATLGEKVTMSCRASSNVKYMYWYQQKSGASPKLWIYYTSNLASGVPARFSGSGSGTSYSLTISSVEAEDAATYYCQQFTSSPYTFGSGTKLEIKRAAAGAPVPYPDPLEPRAA

SEQ ID NO: 85 - protein, 122 aa, synthetic construct
DVQLQESGPGLVKPSQSLSLTCTVTGYSITSDYAWNWIRQFPGNKLEWMGYISSGRTSYNPSLKSRISITRDTSKNQFFLQLNSVTTEDTATYYCARHYGNYEGYYYAMDYWGQGTSVTVSS

SEQ ID NO: 86 - protein, 108 aa, synthetic construct
ETTVTQSPASLSMAIGEKVTIRCITSTGIDDDMNWYQQKPGEPPELLISEGNVLRPGVPSRFSSSGYGTDFLFTIENILSEDVADYYCLQTDNLPLTFGSGTKLEIKR

SEQ ID NO: 87 - protein, 249 aa, synthetic construct
QVQLVQSGAEVKKPGASVKVSCKVSGYTFTSYDINWVRQAPGKGLEWMGWIYPGDGSIKYNEKFKGRVTMTVDKSTDTAYMELSSLRSEDTAVYYCARRGEYGNYEGAMDYWGQGTLVTVSSGGGGSGGGGSGGGGSDIQMTQSPSSLSASVGDRVTITCKASQSVDYDGHSYMNWYQQKPGKAPKLLIYAASNLESGVPSRFSGSGSGTDFTLTISSLQPEDFATYYCQQSDENPLTFGGGTKVEIKR

SEQ ID NO: 88 - protein, 249 aa, synthetic construct
DIQMTQSPSSLSASVGDRVTITCKASQSVDYDGHSYMNWYQQKPGKAPKLLIYAASNLESGVPSRFSGSGSGTDFTLTISSLQPEDFATYYCQQSDENPLTFGGGTKVEIKRGGGGSGGGGSGGGGSQVQLVQSGAEVKKPGASVKVSCKVSGYTFTSYDINWVRQAPGKGLEWMGWIYPGDGSIKYNEKFKGRVTMTVDKSTDTAYMELSSLRSEDTAVYYCARRGEYGNYEGAMDYWGQGTLVTVSS

SEQ ID NO: 89 - protein, 261 aa, synthetic construct
MAQVKLQQSGPELVKPGASVKMSCKASGYTFTSYVMHWVKQSNGKSLEWIGTIDPYYGGTSYNQKFKGKATLTVDKSSTTAYIQLKSLTSEDSAVYYCARWDGYYGGFSYWGQGTMVTVSSGGGGSGGGGSGGGGSDIELTQSPAIMSATLGEKVTMSCRASSNVKYMYWYQQKSGASPKLWIYYTSNLASGVPARFSGSGSGTSYSLTISSVEAEDAATYYCQQFTSSPYTFGSGTKLEIKRAAAGAPVPYPDPLEPRAA

SEQ ID NO: 90 - protein, 245 aa, synthetic construct
ETTVTQSPASLSMAIGEKVTIRCITSTGIDDDMNWYQQKPGEPPELLISEGNVLRPGVPSRFSSSGYGTDFLFTIENILSEDVADYYCLQTDNLPLTFGSGTKLEIKRGGGGSGGGGSGGGGSDVQLQESGPGLVKPSQSLSLTCTVTGYSITSDYAWNWIRQFPGNKLEWMGYISSGRTSYNPSLKSRISITRDTSKNQFFLQLNSVTTEDTATYYCARHYGNYEGYYYAMDYWGQGTSVTVSS

SEQ ID NO: 91 - protein, 245 aa, synthetic construct
DVQLQESGPGLVKPSQSLSLTCTVTGYSITSDYAWNWIRQFPGNKLEWMGYISSGRTSYNPSLKSRISITRDTSKNQFFLQLNSVTTEDTATYYCARHYGNYEGYYYAMDYWGQGTSVTVSSGGGGSGGGGSGGGGSETTVTQSPASLSMAIGEKVTIRCITSTGIDDDMNWYQQKPGEPPELLISEGNVLRPGVPSRFSSSGYGTDFLFTIENILSEDVADYYCLQTDNLPLTFGSGTKLEIKR

SEQ ID NO: 92 - protein, 412 aa, synthetic construct
MPLQLLLLLILLGPGNSLQLWDTWADEAEKALGPLLARDRRQATEYEYLDYDFLPETEPPEMLRNSTDTTPLTGPGTPESTTVEPAARRSTGLDAGGAVTELTTELANMGNLSTDSAAMEIQTTQPAATEAQTTQPVPTEAQTTPLAATEAQTTRLTATEAQTTPLAATEAQTTPPAATEAQTTQPTGLEAQTTAPAAMEAQTTAPAAMEAQTTPPAAMEAQTTQTTAMEAQTTAPEATEAQTTQPTATEAQTTPLAAMEALSTEPSATEALSMEPTTKRGLFIPFSVSSVTHKGIPMAASNLSVNYPVGAPDHISVKQCLLAILILALVATIFFVCTVVLAVRLSRKGHMYPVRNYSPTEMVCISSLLPDGGEGPSATANGGLSKAKSPGLTPEPREDREGDDLTLHSFLP

SEQ ID NO: 93 - protein, 817 aa, synthetic construct
MALPVTALLLPLALLLHAARPEQKLISEEDLQVQLVQSGAEVKKPGASVKVSCKVSGYTFTSYDINWVRQAPGKGLEWMGWIYPGDGSIKYNEKFKGRVTMTVDKSTDTAYMELSSLRSEDTAVYYCARRGEYGNYEGAMDYWGQGTLVTVSSGGGGSGGGGSGGGGSDIQMTQSPSSLSASVGDRVTITCKASQSVDYDGHSYMNWYQQKPGKAPKLLIYAASNLESGVPSRFSGSGSGTDFTLTISSLQPEDFATYYCQQSDENPLTFGGGTKVEIKRILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLSRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 94 - DNA, 270 bp, synthetic construct
  source 1..270: mol_type = other DNA
ggagcactgtcctccgaacgtcggagcactgtcctccgaacgtcggagcactgtcctccgaacgtcggagcactgtcctccgaacggagcatgtcctccgaacgtcggagcactgtcctccgaacgactagttaggcgtgtacggtgggaggcctatataagcagagctcgtttagtgaaccgtcagatcgcctggagacgccatccacgctgttttgacctccatagaagacaccgggaccgatccagcctctcgacattgccgccacc

SEQ ID NO: 95 - protein, 113 aa, synthetic construct
EVKLQQSGPSLVEPGASVMISCKASGSSFTGYNMNWVRQNIGKSLEWIGAIDPYYGGTSYNQKFKGRATLTVDKSSSTAYMHLKSLTSEDSAVYYCVSGMKYWGQGTSVTVSS

SEQ ID NO: 96 - protein, 125 aa, synthetic construct
DILLTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKRADAAPTVSIFP

SEQ ID NO: 97 - protein, 262 aa, Homo sapiens
MALPVTALLLPLALLLHAEVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIK

SEQ ID NO: 98 - protein, 122 aa, synthetic construct
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSS

SEQ ID NO: 99 - protein, 107 aa, synthetic construct
DIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIK

SEQ ID NO: 100 - protein, 492 aa, synthetic construct
MALPVTALLLPLALLLHAEVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKRAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRS

SEQ ID NO: 101 - protein, 6 aa, synthetic construct
HHHHHH